(19) United States
(12) Patent Application Publication
(10) Pub. No.: US 2021/0299150 A1
(43) Pub. Date: Sep. 30, 2021

(54) AP39 IMPACT ON MITOCHONDRIA, METABOLISM, GENE TRANSCRIPTION REGULATION, CARDIOTOXICITY PROTECTION AND CANCER TREATMENT

(71) Applicant: Sudhakar Veeranki, Louisville, KY (US)

(72) Inventor: Sudhakar Veeranki, Louisville, KY (US)

(21) Appl. No.: 17/217,462

(22) Filed: Mar. 30, 2021

Related U.S. Application Data
(60) Provisional application No. 63/002,095, filed on Mar. 30, 2020.

(51) Int. Cl. A61K 31/67; A61K 31/704; C12Q 1/6886; A61K 31/095

(52) U.S. Cl. CPC A61K 31/67; A61K 31/095; C12Q 1/6886; A61K 31/704

(57) ABSTRACT

A method of predicting and treating doxorubicin-resistant cancer including determining expression of Myc-regulated genes and MPST transcription levels. The method further includes classifying the cancer as doxorubicin resistant when either Myc transcription is higher than IMR-32 transcription or MPST transcription levels are above a predetermined amount. The method further includes administering an effective amount of AP39 for ameliorating the doxorubicin-induced cardiotoxicity by combing doxorubicin with AP39.

This application claims the benefit of U.S. Provisional Patent Application Ser. No. 63/002,095, filed on Mar. 30, 2020, the disclosure of which is incorporated herein by reference.

### TECHNICAL FIELD

This disclosure relates to the use of AP39 for the treatment of mitochondrial diseases, cardiotoxicity, and cancer. This disclosure further relates to a method of treating cancer that involves administering thiolactate to a subject in need thereof. This disclosure further provides a method of predicting and treating doxorubicin resistant cancers.

### BACKGROUND

Use of the highly effective broad spectrum anti-cancer chemotherapeutic agent, doxorubicin (Dox), induces Dox-induced cardiotoxicity (DIC) which leads to mortality and morbidity in pediatric cancer survivors. Age, sex, Dox dose and genetic variation modulate susceptibility to DIC. The mechanisms of DIC are not well understood, which limits Dox's broad clinical utilization. Dox treatment alters cardiac metabolism and mitochondrial function, however, underlying mechanisms and prevention strategies have not yet been discovered.

Cancer is one of the world's two leading killers, the other being cardiovascular disease. Among the most potent anti-cancer drugs, Doxorubicin (Dox) is frequently used to treat broad spectrum of cancers and is listed as an essential medicine by the WHO. It is an anthracycline, a group of structurally related chemotherapeutic agents. Paradoxically however, increased cancer survivorship among children led to higher incidence of adverse side effects associated with anti-cancer drug use. Among those side effects the most notable one is DIC. DIC is a leading non-cancer cause of mortality and morbidity among the surviving children with cancers. Anthracycline induced cardiotoxicity (AIC) has two phases: acute and chronic. Cumulative left ventricular systolic dysfunction (LVSD) incidence rate is at 12% within 18 months of Dox treatment and late cardiac dysfunction incidence is at 16% with a sub-clinical disease rate of over 50%. Thus, it led to the adage that ‘today's cancer children are tomorrow's cardiac patients’. Subclinical and chronic forms of DIC further complicate evaluation of cardioprotectants. The only clinically approved drug for DIC, dexrazoxane (Dzx), is currently recommended for only women with breast cancer receiving a cumulative dox dose of above 300 mg/m². Thus, there is no clinically approved cardioprotectant for nearly all of the cancer patients who are undergoing Dox therapy.

Although various prominent mechanisms such as enhanced generation of reactive oxygen species (ROS) and inhibition of topoisomerase 2 have been proposed the exact mechanisms of DIC are unknown and may be multifactorial in nature. Thus far, there is no effective clinical translation that prevents DIC without reducing anticancer efficiency. Moreover, it was recently proposed that sensitivity to anthracycline induced cardiotoxicity (AIC) has a genetic basis, further stressing the need for evaluation of drugs on genetically diverse individuals in a pre-clinical setting. Recently it was demonstrated that hiPSC-CMs replicate many features of DIC. Apart from genetic factors, other predictors of DIC risk are cumulative dosage of Dox, age, endothelial damage, sex and cardiovascular risk factors. Both pediatric and geriatric patients exhibit greater risk for AIC. Female sex is a risk factor in pediatric cancers but may not be the case in the adult cancers. However, no known mechanism explains enhanced susceptibility to DIC by most of these risk factors.

Although the mechanisms are not completely known, the gaseous signaling molecule with antioxidant properties, H₂S, confers protection against DIC. However, untargeted/generic H₂S donors were used in these studies with higher dose ranges. Given the narrow therapeutic index and pleiotropic nature of H₂S signaling, it is hard to clinically translate generic H₂S donors that do not have determined organelle targets. H₂S is actively produced in mitochondria through the mitochondrial localized enzyme, MPST, suggesting a critical role for H₂S in mitochondrial function. Given that Dox primarily affects mitochondrial function, metabolism, biogenesis and ROS production, it is necessary to understand the role of mitochondrial-targeted H₂S. Furthermore, Dox-mediated downregulation of H₂S production was demonstrated to be a Dox resistance mechanism developed by certain resistant tumors and H₂S supplementation potentiated anti-cancer effectiveness of Dox. As H₂S activates AMPK (FIGS. 7A and B) and AMPK activation is an anti-cancer strategy, the instant data may in part explain the observed enhanced Dox anti-cancer potency with H₂S addition. The sum of these findings related to H₂S treatment in heart and cancer therapies suggests that H₂S supplementation with Dox could be the drug combination that delivers cancer potency while minimizing cardiac damage. However, it is currently unclear the impact of targeted H₂S delivery on cardiac transcriptome and metabolome, which is needed for its clinical translation. Mitochondria targeting H₂S donor, AP39, was studied in different contexts including as a cardioprotectant before, but neither tested against DIC nor its effects are completely known in heart.

Max network comprises a group of nuclear transcriptional factors (includes c-Myc, n-Myc, and l-Myc) that regulate critical cell processes such as cell survival, growth, cell division and metabolism. Myc proteins form obligate heterodimers with Max permitting their DNA binding at specific sequences, thus forming higher order activation or repression complexes leading to gene expression regulation. In the absence of Max, Myc forms weak homodimers with weak ability to bind to DNA. c-Myc regulates cardiac metabolism and mitochondrial biogenesis in stress states. c-Myc is instrumental in upregulating glucose metabolism and mitochondrial biogenesis in adult mice hearts in response to ischemic injury. Although it was shown that c-Myc is not needed for normal function and development of heart, controlled cardiomyocyte c-Myc overexpression confers survival advantages and compensates for the loss of other Myc related proteins. Moreover, c-Myc depletion in endothelial lineage resulted in heart defects suggesting the critical role of c-Myc in endothelial cells as well. However, the impact of Dox on the Myc/Max regulated metabolome and transcriptome in different cardiac cell-types (cardiomyocyte vs endothelial) have not been assessed. Recent findings also indicated that Myc and mitochondria exhibit mutual positive regulation, which is crucial for survival and Myc intimately regulates mitochondrial biogenesis and metabolism in diverse systems. Given the significance of Myc/Max in conferring cell survival during stress, it is critical to know the impact of Dox/AP39 on the Myc/Max regulated transcriptome and metabolome.

In addition to Myc/Max, AMPK also regulates mitochondrial biogenesis and metabolism. AMPK participates in mitochondrial quality control and mitophagy, thus it is needed for eliminating damaged mitochondria in stress situations. Further, during stress, AMPK, a metabolic master switch, promotes energy generating catabolic pathways and halts energy consuming anabolic pathways (synthesis of lipids, proteins and glycogen), thus, conferring cell survival. Interestingly, studies also suggested that Myc and AMPK engages in significant cross-talk to regulate mitochondrial function and metabolism. However, the impact of H₂S/Dox on regulation of metabolism, Myc/Max transcription and AMPK is unknown.

Cancer survivors suffer from cancer drug-mediated cardiotoxicity, which leads to mortality and morbidity¹. Dox causes potent cardiotoxicity and death in childhood cancer survivors²⁻⁵. Although Dox is the first line of treatment for neuroblastoma, DIC precludes its use in higher doses, which also leads to tumor resistance to Dox⁶⁻¹². The only clinically approved drug for DIC, dexrazoxane (Dzx), is currently recommended only for women with breast cancer receiving a cumulative Dox dose of above 300 mg/m^{2 13,14}, a dose that is too high for cancer patients. Thus, there is no clinically approved cardioprotectant for nearly all of cancer patients. Also, high-risk neuroblastoma causes drug resistance and severe mortality. Hence, there is a need for the identification of novel drugs or drug combinations. Ideally, a drug is needed that can protect the heart against DIC without reducing the anticancer efficacy of Dox and/or with the ability to reduce half-maximal inhibitory concentration (IC₅₀) of Dox.

Drug resistance and drug-induced cardiotoxicity are responsible for significant mortality in neuroblastoma (NB) patients and other cancer (breast, lung, renal) patients. Cancer survivors suffer from Doxorubicin (Dox)-induced cardiotoxicity (DIC), which leads to mortality and morbidity. Although Dox is the first line of treatment for neuroblastoma and other cancers, DIC precludes its use in higher doses, which also leads to tumor resistance to Dox. The only clinically approved drug for DIC, dexrazoxane (Dzx), is currently recommended only for women with breast cancer receiving a cumulative Dox dose of above 300 mg/m², a dose that is too high for any cancer patient. Thus, there is no clinically-approved cardioprotectant for nearly all of the cancer patients. The goal of the instant invention is to: 1) reduce cancer mortality and 2) improve quality of life in cancer survivors. Also, high-risk neuroblastoma causes drug resistance and severe mortality. Hence, there is a need for the identification of novel drugs or drug combinations. Ideally, a drug is needed that can protect the heart against DIC without reducing the anticancer efficacy of Dox and/or with the ability to reduce half-maximal inhibitory concentration (IC₅₀) of Dox.

H₂S, an endogenous gaseous signaling molecule, can be a cytoprotective or toxic depending on its levels. The three key enzymes that produce endogenous H₂S are CBS, CSE/CTH, and MPST. MPST is localized in mitochondria. H₂S supplementation has been shown to be a cardioprotective approach. Nearly all the cancer cells upregulate the three-key endogenous H₂S producing enzymes. Especially the MPST is elevated nearly 2- to 4-fold (Cancer Cell Line Encyclopedia, Broad Institute) in 98% of the cancer cell lines belonging to 40 different types of cancers. Thus, cancer cells can be made prone to H₂S poisoning through external H₂S supplementation using H₂S donors, as they already elevated their H₂S levels more than normal tissues, such as in the heart. H₂S supplementation potentiated the anti-cancer effectiveness of Dox. Mitochondria targeting H₂S donor, AP39, was studied in different contexts including as a cardioprotectant before, but it was neither tested against DIC nor its effects completely known in heart. Also, AP39 anti-cancer potential and its impact on Dox anticancer efficacy are not evaluated especially against neuroblastoma, breast, lung, renal and potentially other cancers. Although MYCN is frequently amplified in NB, whether H₂S production regulates Myc transcription and whether elevated Myc transcription and H₂S production leads to Dox resistance is unknown.

### BRIEF DESCRIPTION OF THE DRAWINGS

The presently-disclosed subject matter will be better understood, and features, aspects and advantages other than those set forth above will become apparent when consideration is given to the following detailed description thereof. The patent or application file contains at least on drawing executed in color. Copies of this patent or patent application publication with color drawing(s) will be provided by the Office upon request and payment of the necessary fee. Such detailed description makes reference to the following drawings, wherein:

FIG. 1 shows a schematic describing cellular pathways impacted by AP39 with respect to doxorubicin induced cardiotoxicity.

FIG. 2A shows Dox treatment resulted in significant reduction in MPST. Western images showing MPST levels after Dox treatment (A and B) in H9C2 cells.

FIG. 2B shows Dox treatment resulted in significant reduction in MPST. Western image quantification showing MPST levels after Dox treatment in H9C2 cells. (n=3, p<0.01; * vs Cont.).

FIG. 2C shows MPST, but not CSE, is exclusively localized in purified mitochondrial fractions from mouse hearts. (n=¾, p<0.01; * vs Cont.).

FIG. 3A shows amelioration of DIC in vivo, treatment schedule.

FIG. 3B shows amelioration of DIC in vivo, heart weight measurements. p<0.05 or 0.01 (2 symbols); * vs Cont; # vs Dox.

FIG. 3C shows amelioration of DIC in vivo, echocardiography results: ejection fraction. p<0.05; * vs Cont; # vs Dox.

FIG. 3D shows amelioration of DIC in vivo heart echocardiography data various heart functional data comparison between groups. p<0.05 (red); * vs Cont.

FIG. 4A shows Amelioration of Dox cardiomyopathy in vivo. Histological sections depicting heart pathology after different the treatments. cell infiltration into tissues.

FIG. 4B shows Amelioration of Dox cardiomyopathy in vivo. Histological sections depicting heart pathology after different the treatments. tissue fibrosis is shown.

FIG. 4C shows Amelioration of Dox cardiomyopathy in vivo. Quantification of cell infiltration into tissues.

FIG. 4D shows Amelioration of Dox cardiomyopathy in vivo. Quantification of tissue fibrosis is shown. p<0.05 or 0.01 (2 symbols); * vs Cont; # vs Dox.

FIG. 5A shows H9C2 (Rat cardiomyoblasts) exhibited ameliorated cell death (crystal violet staining).

FIG. 5B shows H9C2 (Rat cardiomyoblasts) _{exhibited} ameliorated cell death (crystal violet staining). Quantification at various doses. p<0.05 or 0.01 (2 symbols) or p<0.001 (3 symbols); * vs Cont; # vs Dox. n=3.

FIG. 5C shows H9C2 (Rat cardiomyoblasts) exhibited ameliorated cell death reduced cleaved caspase 3 and PARP levels, attenuated stress signaling activation (phospho ERK1/2(T202/Y204) and phospho p38 (T180/Y182))

FIG. 5D shows H9C2 (Rat cardiomyoblasts) exhibited ameliorated cell death reduced cleaved caspase 3 and PARP levels, attenuated stress signaling activation (phospho ERK1/2(T202/Y204) and phospho p38 (T180/Y182)) quantification. p<0.05 or 0.01 (2 symbols) or p<0.001 (3 symbols); * vs Cont; # vs Dox. n=3.

FIG. 5E H9C2 (Rat cardiomyoblasts) exhibited ameliorated ROS production in response to Dox and AP39 co-treatment. p<0.05 or 0.01 (2 symbols) or p<0.001 (3 symbols); * vs Cont; # vs Dox. n=3.Cell death.

FIG. 5F H9C2 (Rat cardiomyoblasts) exhibited ameliorated ROS production response to Dox and AP39 co-treatment.

FIG. 6A shows AP39 treatment causes mitochondrial fission and improves quality control in correlation with H₂S presence. AP39 treatment resulted in H₂S production enhancement (yellow arrows pointing dotted pattern of H₂S presence) and increased mitochondrial fission. Note that white arrow heads and yellow arrows compare and contrast no-H₂S: fusion and H₂S-presence: fission scenarios in the same image AP39 treatment lane. Dox treatment resulted in lower H₂S levels and fusion.

FIG. 6B shows AP39 treatment causes mitochondrial fission and improves quality control in correlation with H₂S presence. AP39 treatment resulted in increased mitochondrial fission and is dominant to Dox mediated mitochondrial fusion. Dox treatment caused auto fluorescence nuclei (an indicator of Dox treatment).

FIG. 6C shows AP39 treatment causes mitochondrial fission and improves quality control in correlation with H₂S presence. Quantification of mitochondrial H₂S levels. n=3; p<0.05 or 0.001 (3 symbols); * vs Cont or Dox; # vs Dox.

FIG. 6D AP39 treatment causes mitochondrial fission and improves quality control in correlation with H₂S presence. Quantification of mitochondrial H₂S levels. n=3; p<0.05 or 0.001 (3 symbols); * vs Cont or Dox; # vs Dox.

FIG. 6E AP39 treatment causes mitochondrial fission and improves quality control in correlation with H₂S presence. Quantification of mitochondrial length after treatments. n=3; p<0.05 or 0.001 (3 symbols); * vs Cont or Dox; # vs Dox.

FIG. 7A shows Mitochondrial biogenesis and quality control were improved after AP39 treatment. AP39 enhanced AMPK activation and produced shorter isoform of Opa1, i.e. no longer isoform of Opa1. Note AMPK substrate ACC phosphorylation

FIG. 7B shows Mitochondrial biogenesis and quality control were improved after AP39 treatment. AP39 enhanced AMPK activation and produced shorter isoform of Opa1, i.e. no longer isoform of Opa1. Quantification of protein changes as in FIG. 7A. n=3; p<0.05; * vs Cont; # vs Dox.

FIG. 7C shows Mitochondrial biogenesis and quality control were improved after AP39 treatment. AP39 enhanced AMPK activation (A and B) and produced shorter isoform of Opa1, i.e. no longer isoform of Opa1. Oxygen consumption rates (OCR) with different treatments. AP39 treatment did not improve OCR but enhances overall energy efficiency through Opa1. n=3; p<0.05; * vs Cont; # vs Dox. Note AMPK substrate ACC phosphorylation.

FIG. 8A shows RNAseq results from the treatments in H9C2 cell line. The coverage among groups are indicated.

FIG. 8B shows RNAseq results from the treatments in H9C2 cell line. The correlations among groups are indicated.

FIG. 8C shows RNAseq results from the treatments in H9C2 cell line. The heatmap depicting the changes in top 250 Myc transcriptional targets among different groups are also presented. The AP39 mediated modulation of Myc transcription is crucial in countering Dox toxicity.

FIG. 8D shows RNAseq results from the treatments in H9C2 cell line. The differentially expressed gene counts between groups are indicated.

FIG. 8E shows RNAseq results from the treatments in H9C2 cell line. The principal component analysis based relative relationship among different groups are also presented.

FIG. 9A shows AP39 prevents Dox inhibition on Max and metabolism. Heatmap showing top 25 Myc regulated transcript levels related to mitochondrial metabolism between groups.

FIG. 9B shows AP39 prevents Dox inhibition on Max and metabolism. Western images showing the significant reduction in Max levels, a DNA-binding-mediating partner of Myc Note that AP39 significantly ameliorated levels of citrate synthase (CS), a rate limiting enzyme for entry into TCA cycle. n=3; p<0.05; * vs Cont; # vs Dox.

FIG. 9C shows AP39 prevents Dox inhibition on Max and metabolism. Quantification of changes as in FIG. 9B. n=3; p<0.05; * vs Cont; # vs Dox.

FIG. 10A shows AP39 causes significant reversal of abnormal metabolism induced by Dox treatment at 16 hrs and 24 hrs. Stable isotope (13C glucose) tracer studies. Heatmap.

FIG. 10B shows AP39 causes significant reversal of abnormal metabolism induced by Dox treatment at 16 hrs and 24 hrs. Stable isotope (13C glucose) tracer studies. Principal component analysis.

FIG. 10C shows AP39 causes significant reversal of abnormal metabolism induced by Dox treatment at 16 hrs and 24 hrs. Quantification of polar metabolites through GC-MS, A heatmap is presented.

FIG. 10D shows AP39 causes significant reversal of abnormal metabolism induced by Dox treatment at 16 hrs and 24 hrs. Quantification of glutamine, glucose and lactate in the cell culture supernatants at 16 and 24 hrs. n=3; p<0.05 or 0.01 (2 symbols); * vs Cont ; # vs Dox at 16 hrs; and $ vs Cont; \hat{ } vs Dox at 24 hrs.

FIG. 11A shows AP39 protects hiPSC-CMs from DIC. AP39 significantly attenuated DIC induced apoptosis (Annexin V) and mitochondrial ROS production (MitoSox Red). Note that Dox image has lower number of cells than Dox+Ap39 group, yet resulted in higher overall staining pattern.

FIG. 11B shows AP39 protects hiPSC-CMs from DIC. AP39 significantly attenuated DIC induced apoptosis (Annexin V) and mitochondrial ROS production (MitoSox Red). Quantification of results as in FIG. 11A. n=1 with 3 replicates; p<0.05 or 0.01 (2 symbols); * vs Cont; # vs Dox.

FIG. 12 shows a schematic describing AP39 activity on cancer and cardiotoxicity.

FIG. 13A shows Higher MPST levels in 38 out of 39 different human neuroblastoma cell lines compared to fetal brain (data mining was done using the reported RNAseq data¹⁵).

FIG. 13B shows Higher MPST levels in 38 out of 39 different human neuroblastoma cell lines compared to fetal brain (data mining was done using the reported RNAseq data¹⁵). H₂S producing MPST and CBS are highly correlated with MYCN levels.

FIG. 14 shows Selection of cell lines (among 39 neuroblastoma cell lines and prediction of AP39 response based on the Myc driven transcription. MPST levels were also included. Extreme left: higher Myc transcription and extreme right: lower Myc transcription. A Dox resistance prediction model; higher Myc transcription and higher MPST/H₂S levels indicate Dox resistance (extreme left, for example SK-N-BE(2)).

FIG. 15 shows AP39 use reduces the IC values of Dox against SK-N-BE(2) NB cell line, a highly Dox resistant cell line. Images showing effects of AP39 and Dox. Right, quantification of cell viability at 24 hrs after treatments. p<0.05 or 0.01 (2 symbols); * vs Cont.

FIG. 16 shows the mRNA expression profile of MPST in several cancer cell lines (Cancer Cell Line Encyclopedia, Broad Institute).

FIG. 17 shows images of SKNBE(2) cells treated with control, AP39 (AP), AP39 and Doxorubicin, Doxorubicin alone, Thiolactate alone, AP39 and Thiolactate, and Doxorubicin and Thiolactate.

FIG. 18 shows images of renal cancer cells 786-O treated with control, AP39 (AP), AP39 and Doxorubicin, Doxorubicin alone, Thiolactate alone, AP39 and Thiolactate, and Doxorubicin and Thiolactate.

FIG. 19 shows images of A549 lung cancer cells treated with control, AP39 (AP), AP39 and Doxorubicin, Doxorubicin alone, Thiolactate alone, AP39 and Thiolactate, and Doxorubicin and Thiolactate.

FIG. 20 shows images of H1299 lung cancer cells treated with control, AP39 (AP), AP39 and Doxorubicin (Dox), Doxorubicin alone.

FIG. 21 shows MTT viability assays of SK-N-BE(2), 786-O, A549, H1299 cells treated with control, AP39 (AP), AP39 and Doxorubicin (Dox), or Doxorubicin alone.

FIG. 22 shows MTT viability assays of H1299 cells treated with control, AP39 (AP), AP39 and Doxorubicin, Doxorubicin alone, Thiolactate alone, AP39 and Thiolactate, and Doxorubicin and Thiolactate.

FIG. 23 shows mRNA expression profile of LDHA (lactate dehydrogenase A) in several cancer cell lines (Cancer Cell Line Encyclopedia, Broad Institute). Thiolactate a drug to target cancer cells that express increased LDH and H₂S levels (FIG. 16).

FIG. 24 shows activity of phosphorylated ERK1/2 of SKNBE(2) cells treated with control, AP39, AP39 and Doxorubicin, Doxorubicin alone, AP39 and Thiolactate, and Doxorubicin and Thiolactate.

FIG. 25 shows images of SKNBE(2) cells treated with control, AP39, AP39 and Doxorubicin, Doxorubicin alone, AP39 and Thiolactate, and Doxorubicin and Thiolactate.

FIG. 26 shows images of breast cancer MCF7 cells treated with control, AP39 (AP), AP39 and Doxorubicin, Doxorubicin alone, Thiolactate alone, AP39 and Thiolactate, and Doxorubicin and Thiolactate.

FIG. 27 shows MTT viability assays and crystal violet assays for breast cancer MCF7 cells treated with control, AP39 (AP), AP39 and Doxorubicin, Doxorubicin alone, AP39 and Thiolactate, and Doxorubicin and Thiolactate.

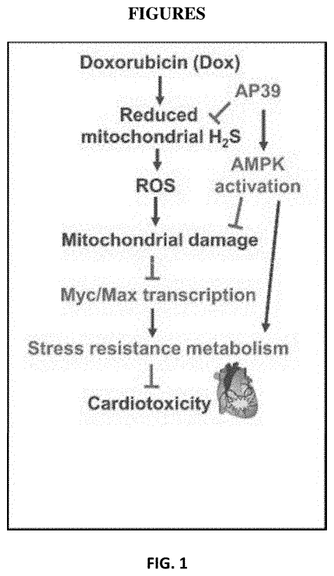

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter generally relates to a method of treating Doxorubicin mediated organ toxicity comprising: Administering AP39 to a subject in need thereof. In some embodiments, the organ is the heart. In further embodiments of the invention, AP39 is administered at a dose of between about 0.321 mg/kg to about 0.642 mg/kg. In other embodiments of the present invention, AP39 is administered at a dose of at least about 20 micromolar.

Another embodiment of the present invention relates to a method of treating cancer comprising administering AP39 to a subject in need thereof. In some embodiments, doxorubicin is further co-administered to the subject. In further embodiments, the cancer is lung cancer, neural cancer, breast cancer, or renal cancer.

One embodiment of the present invention is a method of method of treating cancer comprising administering Thiolactate to a subject in need thereof. Further embodiments comprise administering AP39 to the subject in addition to thiolactate. In some embodiments, doxorubicin is further co-administered to the subject. In further embodiments, the cancer is lung cancer, neural cancer, breast cancer, or renal cancer.

Another embodiment of the present invention relates to a method of predicting and treating doxorubicin resistant cancer comprising: (a) determining expression of Myc-regulated genes and MPST transcription levels; (b) classifying the cancer as doxorubicin resistant when either Myc transcription is higher than IMR-32 transcription or MPST transcription levels are above a predetermined amount; and (c) administering an effective amount of AP39. In a further embodiment, the cancer is neuroblastoma

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims. Thus, for example, reference to “a biomarker” includes a plurality of such biomarkers, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, width, length, height, concentration or percentage is meant to encompass variations of in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the term “subject” refers to a target of administration. The subject of the herein disclosed methods can be a mammal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A “patient” refers to a subject afflicted with a disease or disorder. The term “patient” includes human and veterinary subjects.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term neural cancer includes all cancers consisting of or originating from nervous tissue. Examples include brain cancers such as neruoblastoma, glioma, astrocytoma, meningioma, and oligodendroglioma.

As used herein, SK-N-BE(2) cells are known models of neural cancer and epithelial cancers.

The presently-disclosed subject matter is further illustrated by the following specific but non-limiting examples. The following examples may include compilations of data that are representative of data gathered at various times during the course of development and experimentation related to the present invention.

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter is further illustrated by the following specific but non-limiting examples. The following examples may include prophetic examples, notwithstanding the numerical values, results and/or data referred to and contained in the examples. The following examples may include compilations of data that are representative of data gathered at various times during the course of development and experimentation related to the present invention.

### EXAMPLES

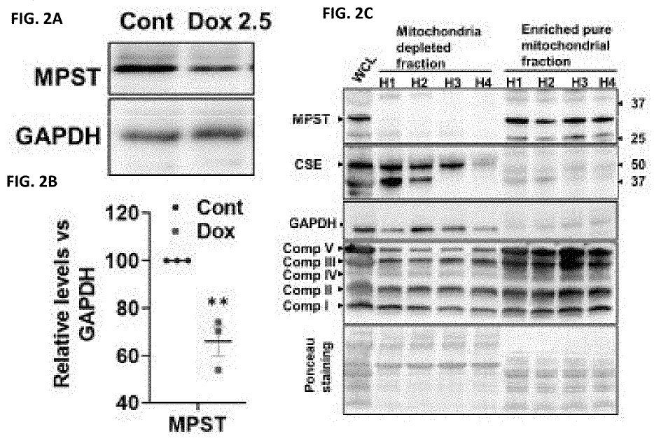

Doxorubicin inhibits mitochondrial H₂S producing enzyme, MPST, levels (FIG. 2): Upon Dox treatment there was significant reduction in MPST in H9C2 cells, suggesting that Dox inhibits H₂S production in mitochondria (FIGS. 2A and B). MPST localization was confirmed in mitochondria after isolating pure mitochondria from mouse hearts (FIG. 2C). Another H₂S producing enzyme, CSE (Cystathionine gamma-lyase), was detectable only in the cytosolic fractions (FIG. 2C).

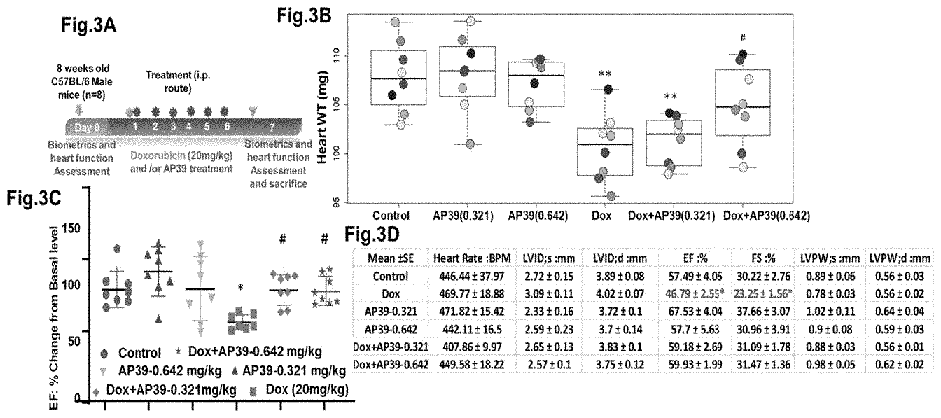

AP39 treatment ameliorated DIC in mouse models in a dose dependent manner. DIC was induced by giving a single dose of Dox i.p. (FIG. 3A) as reported before⁴⁸. AP39 was given for six consecutive days starting with Dox treatment. two different doses of AP39 were tested. The higher dose (0.642 mg/kg) was effective in countering heart weight decline seen during Dox treatment. However, both doses of AP39 were effective in ameliorating left ventricular dysfunction. The results indicated that AP39 is effective in preventing DIC (FIGS. 3C and D).

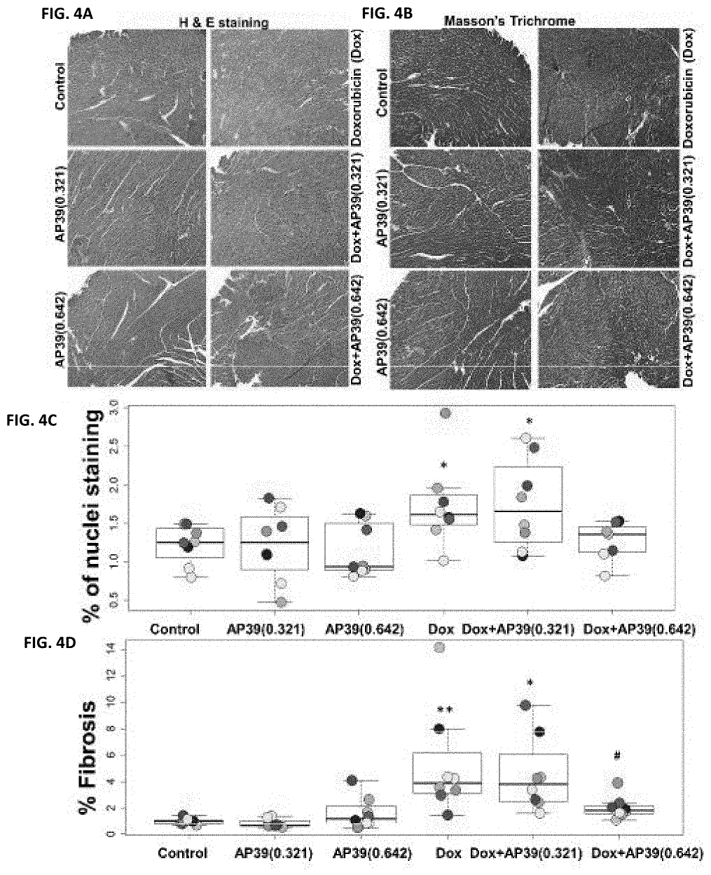

AP39 ameliorated cardiac fibrosis in a dose dependent manner. Cell infiltration (total nuclei, FIGS. 4A, C) and cardiac fibrosis (blue, FIGS. 4B, D) was measured in histological sections to determine the extent that AP39 treatment attenuated DIC. Results (FIG. 4) suggest that only the higher dose of H₂S prevented Dox induced fibrosis and cell infiltration. The lower AP39 dose was able to improve the heart function (FIG. 3) but not cardiac pathology (FIG. 4). It is possible that lower doses of AP39 might have induced favorable metabolic changes which are enough to improve heart function but not sufficient to protect from cardiac cell death after Dox. These findings needs to be confirmed in a larger study.

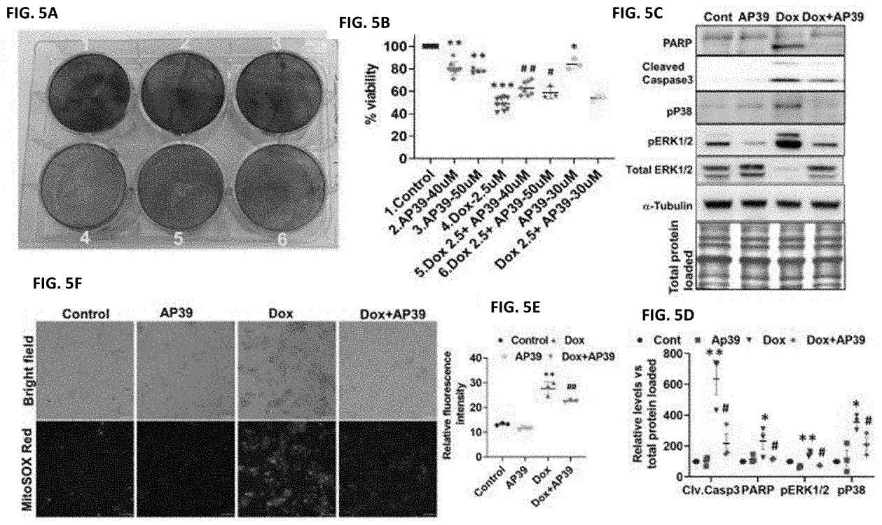

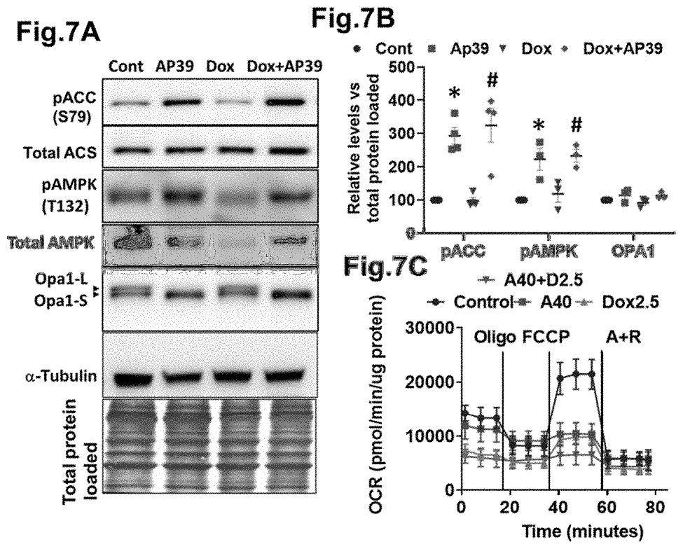

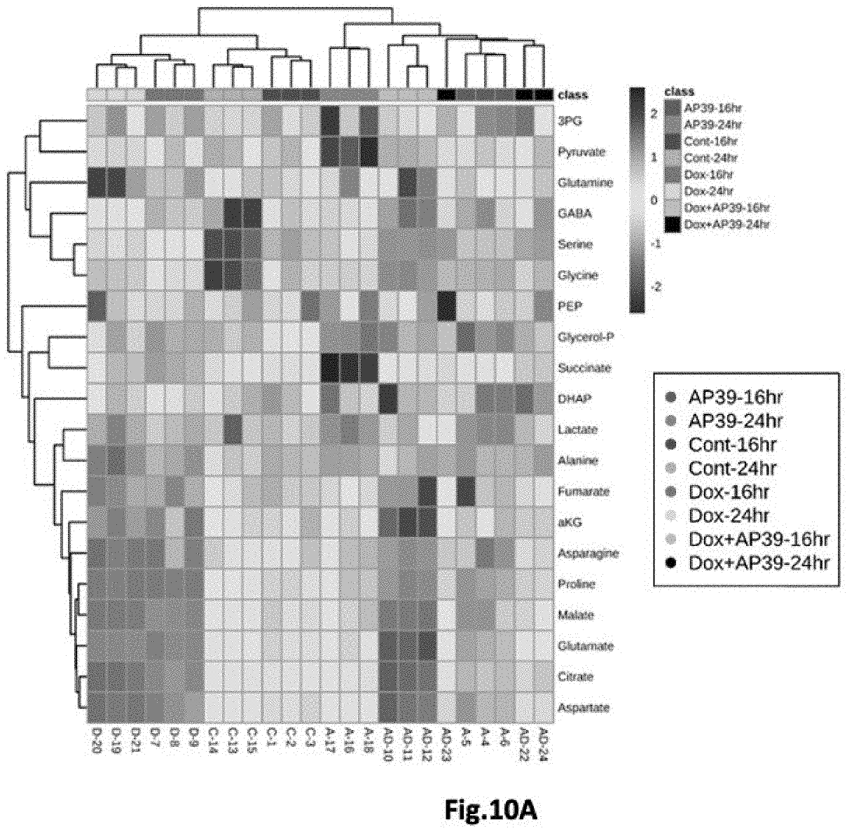

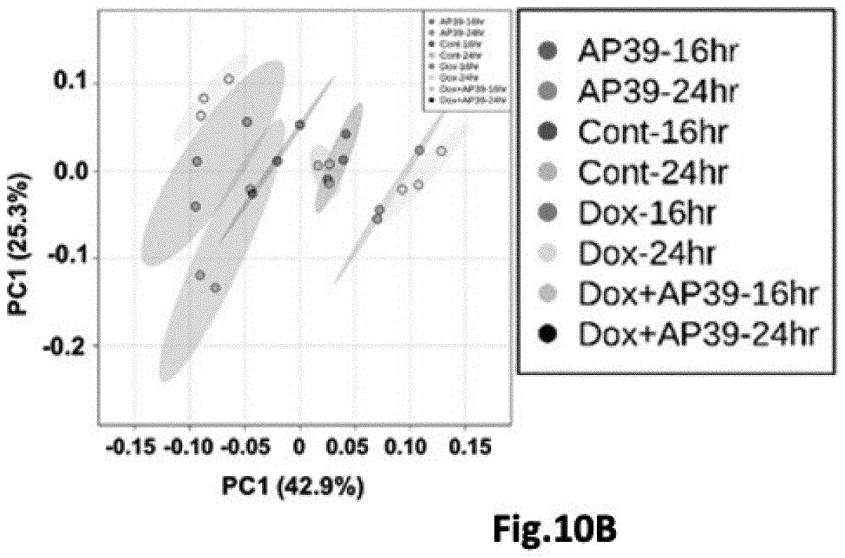

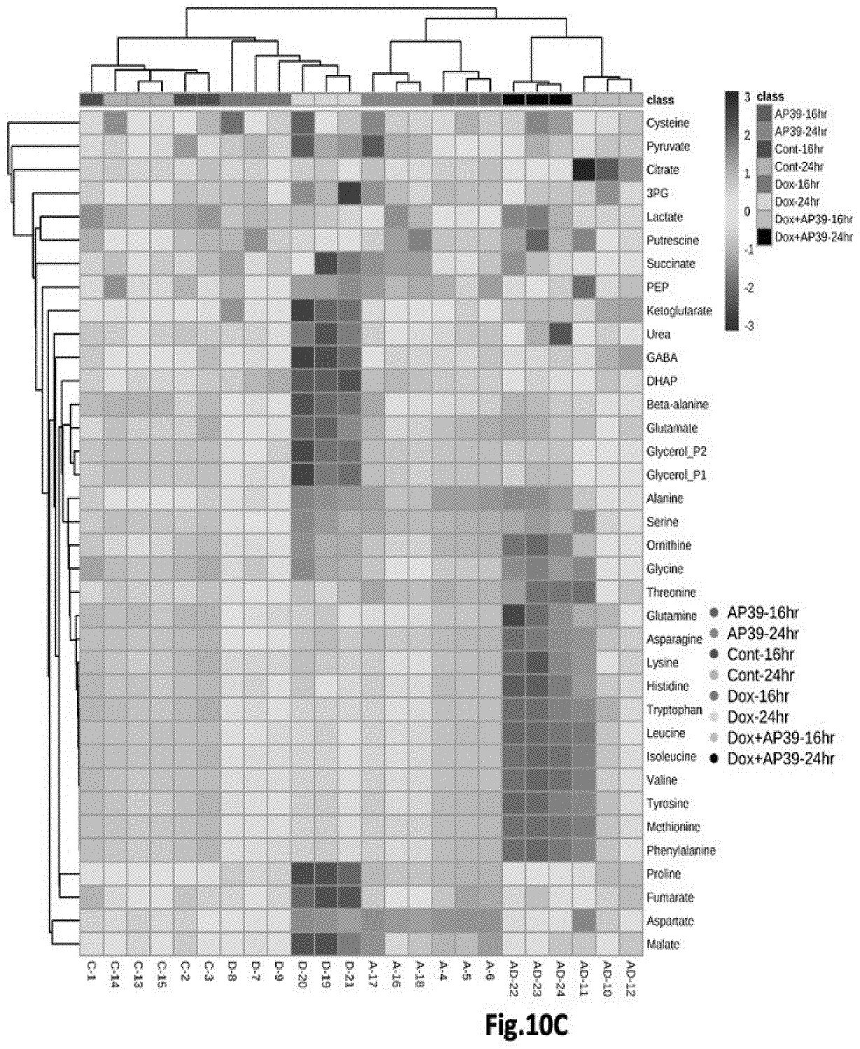

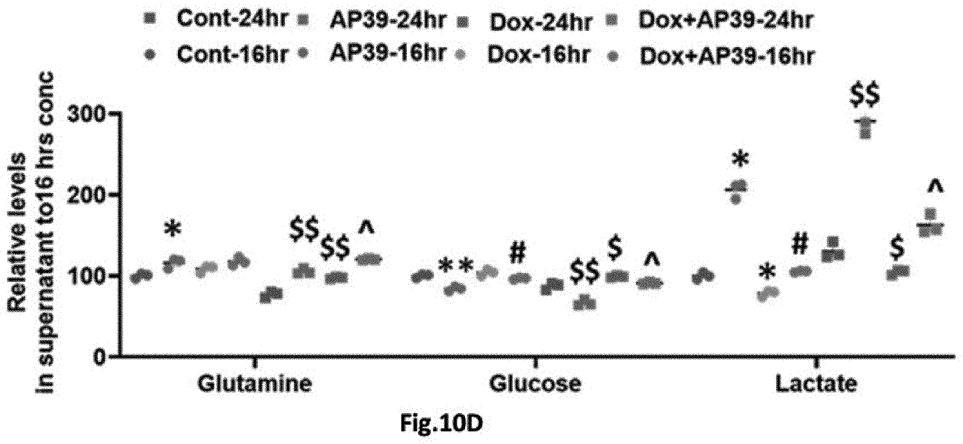

AP39 treatment also ameliorated Dox toxicity in a model of Rat cardiomyoblast cell line, H9C2. AP39 treatment also resulted in significant protection in vitro as assayed by crystal violet staining. There was a narrow window of protection, as 30 μM and 50 μm doses were not as strong as the 40 μM dose (FIGS. 5A and B) in conferring protection. Further, there were significantly reduced PARP and caspase 3 cleavage after AP39 treatment (FIGS. 5C and D). This reduction in apoptotic markers after AP39 were also correlated with reduced stress signaling (activated ERK1/2 and p38 kinases, FIGS. 5C and D), which was in contrast to the Dox only group. In line with reduced apoptotic markers and reduced activation of stress signaling, there was also significant amelioration of ROS in mitochondria as evidenced by mitochondrial oxidative stress sensitive dye, MitoSox Red (FIGS. 5E and F). Note that Dox caused an apoptotic phenotype in correlation with ROS levels (FIG. 5F, Dox lane). This in vitro AP39 protection is modest when compared to the in vivo scenario. This difference may be due to rapid clearance of toxic metabolites (for instance, higher lactate production, FIG. 10D) and better oxygen supply (AP39 stimulated OCR, FIG. 7C) in vivo and lack of such sophistication in vitro. Nonetheless, these findings strongly reiterated that AP39 mediates protection against DIC (FIGS. 3 and 4) and suggests that AP39/H₂S potently inhibits oxidative stress induced by Dox treatment.

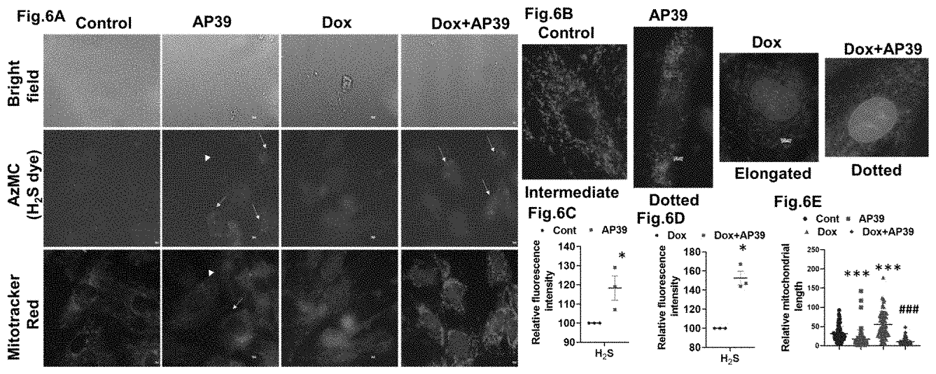

Mitochondrial fission was correlated with H₂S levels. To investigate the impact of Dox on mitochondrial biogenesis and mitochondrial H₂S levels, H9C2 cells were used in combination with H2S sensitive dye AzMAC and mitotracker Red dye as reported previously⁸. The results (FIG. 6) suggested that Dox significantly reduced mitochondrial H₂S production, as also noted in FIG. 2A with MPST. Further, H₂S levels (yellow arrows) highly correlated with a dotted appearance of mitochondria, implying that H₂S causes mitochondrial fission, which is a prerequisite for eliminating damaged mitochondria⁴⁹. Interestingly, Dox treatment caused the opposite effect, mitochondrial fusion, which was reversed after AP39 treatment. The control group exhibited an intermediate-type. Dox is adversely affecting mitochondrial function by preventing quality control (fused and elongated mitochondria, FIG. 6) and by elevating damaging ROS levels (FIG. 5F). AP39 treatment significantly reversed these two prime changes and thus, protected the mitochondria

Mitochondrial energy efficiency is regulated through accumulation of the shorter form of Opa1 after AP39 treatment. AP39 treatment resulted in elevated amounts of the short form of Opa1 (Opa1-S) at the expense of long form (Opa1-L) (FIGS. 7A and B). It was noted that Opa1-L promotes mitochondrial fusion and Opa1-S promotes energetic efficiency (also observed in FIG. 6). Moreover, Opa1-L accumulation was shown to delay mitophagy, a quality control mechanism. Interestingly it was also observed, in addition to Opa1-S elevation and disappearance of Opa1-L, potent AMPK activation after AP39 treatment (FIGS. 7A and B). Previous studies demonstrated the critical role of AMPK in regulation of mitophagy and thus, its contribution to cell survival. Thus, AP39 treatment led to mitochondrial fission, better quality control through increased mitophagy (increased AMPK activation) and energetic efficiency (short form of Opa1), which were all needed to eliminate Dox damaged mitochondria and to halt the functional decline. Apart from its role in mitophagy and quality control, AMPK activation also induces transcription of nuclear encoded mitochondrial resident proteins. These results suggest improved quality control, efficient energetics and mitochondrial biogenesis.

AP39 treatment suppressed Oxygen Consumption Rate (OCR): AP39 suppressed OCR and was not significantly different in the presence of Dox (FIG. 7C). It is possible that AP39 relies mostly on increased energy efficiency rather than on improving OCR after Dox treatment.

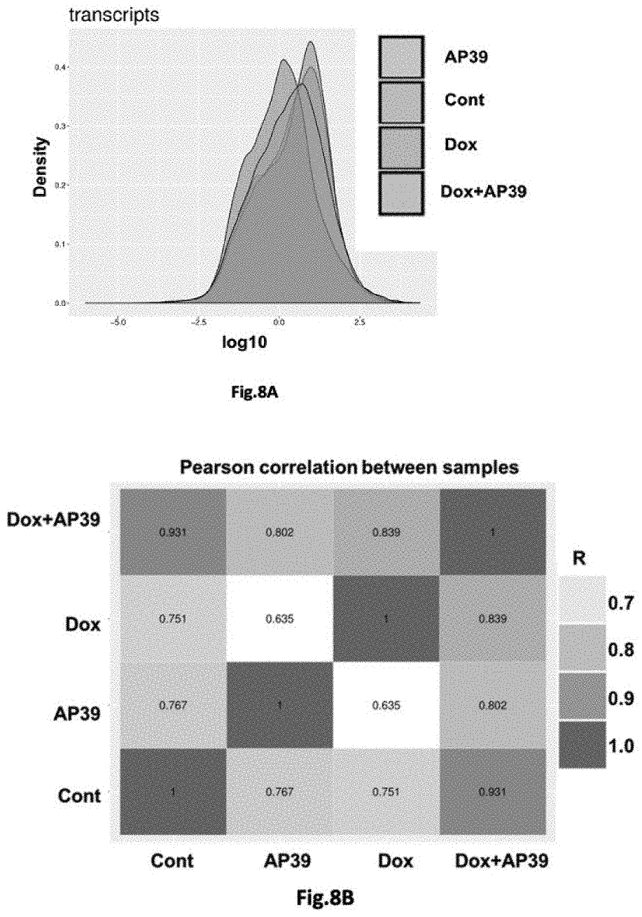

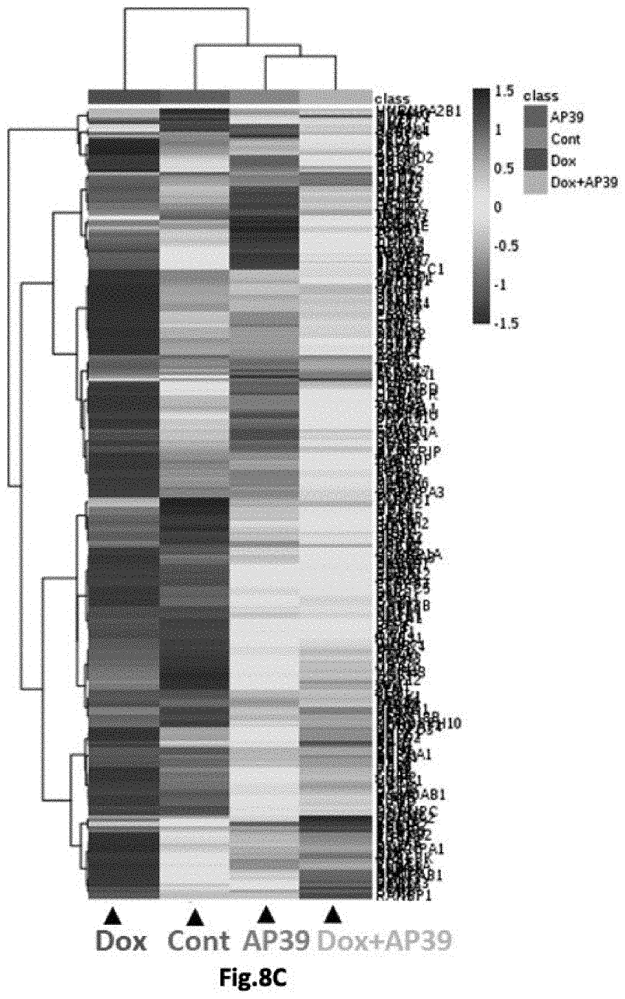

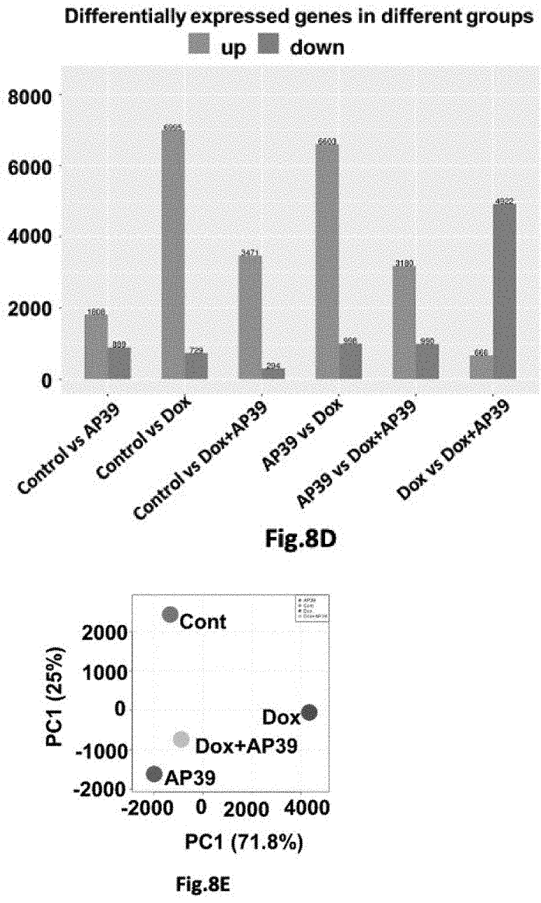

AP39 significantly reversed abnormal transcriptional changes induced after Dox treatment at the global level. Next, to interrogate mechanisms of AP39 protection against DIC, RNAseq analysis was performed. There was sufficient coverage of the whole transcriptome among the groups (FIG. 8A). Whole transcriptome comparisons between groups using Pearson correlation revealed that the AP39+Dox group was much closer to the control group than to the Dox or AP39 groups (FIG. 8B), implying that AP39 caused significant reversal of changes that occurred at transcription with Dox treatment. This is also evident when the up- and down-regulated genes were counted (FIG. 8D). Interestingly, the degree of reversal was much higher when the Myc/Max-regulated transcripts (˜250 known genes) alone were compared (FIG. 8C and E, Heatmap and principal component (PC) analysis). Further, these results suggest that observed variations can be explained by very few significant gene changes (PC1+PC2>90%) and Myc/Max-transcriptional regulation could be one of them.

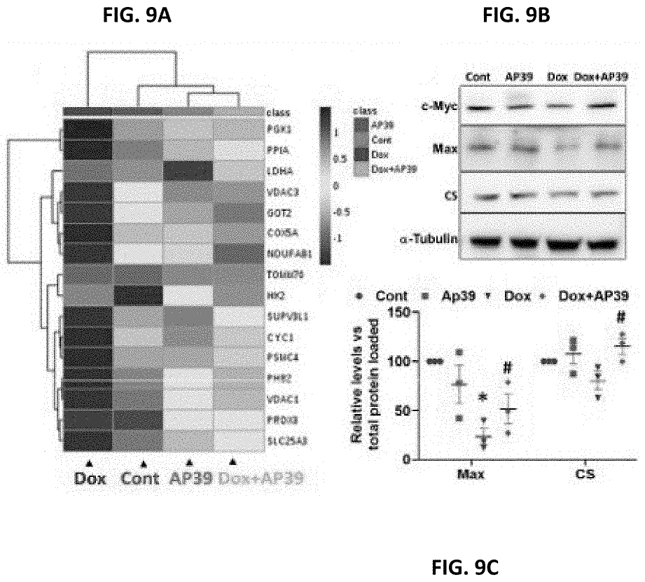

Levels of Max, the DNA-binding-enabling-heterodimer-forming partner for Myc, were reversed with AP39 treatment. As there was overwhelming suppression of Myc transcription (FIG. 8C), the levels of the Myc and its partner Max were verified. In line with RNAseq results, Max protein but not Myc was significantly downregulated after Dox treatment and AP39 restored Max levels (FIGS. 9B and C). These results further confirm that inhibition of Myc/Max transcription is the cause behind Dox toxicity and AP39 ameliorated it. Interestingly, it was also found that Myc regulated nuclear encoded mitochondrial localized gene expressions and stress resistant metabolism regulating gene expressions were also suppressed after Dox treatment (FIG. 9A). These findings strongly suggest that Myc/Max dependent metabolism is derailed after Dox treatment.

AP39 attenuated several abnormal metabolic changes induced by Dox: As Dox attenuated Myc transcription and AP39 restored Myc transcription and activated AMPK, it necessitated exploring associated metabolic changes. To this end, 13C-glucose flux/isotope tracing and quantified metabolites through GC-MS were examined at 16 hrs and 24 hrs after the treatments. Glucose, glutamine and lactate were also measured in supernatants at these time points. Nearly all labeled fractions were significantly different between Dox and AP39 treatments and AP39 reversed many of the Dox induced changes (FIG. 10A). Of note, TCA cycle metabolites (citrate, malate, alpha-ketoglutarate and fumarate) were highly labeled after AP39 treatment with or without Dox (FIG. 10A), indicating active flux through the TCA cycle. Serine and glycine labeling was suppressed after AP39 treatment but were highly labeled in the Dox group (FIG. 10A). With the exception of glycolytic metabolites, the remaining metabolic pathways (poor labeling) were non-operational after Dox treatment (FIG. 10A). The PC analysis also indicated differential grouping, again, suggesting a very significant reversal of Dox-induced metabolic patterns with AP39 treatment (FIG. 10B).

Interestingly, most of the metabolites were present in abundant amounts after 24 hrs of Dox treatment (FIG. 10C). Although AP39 treatment attenuated accumulation of TCA metabolites during Dox treatment, it was not effective in preventing many amino acid accumulations. Suggesting that unlike Dox treatment, AP39 selectively inhibited certain pathways. When considered AP39-mediated activation of AMPK, H₂S is selectively halting energy consuming anabolic pathways, such as protein synthesis (also, lipid, AMPK inhibits ACS, a rate limiting lipid synthesizing enzyme, FIGS. 7A and B), as observed before⁴¹, thus leading to accumulation of amino acids and is enhancing TCA cycle through AMPK activation. The net result is better energy management with AP39/H₂S treatment.

In tandem with isotope tracing and metabolite quantification data, metabolite quantification in supernatants also suggested very significant differences (FIG. 10D). AP39 suppressed glutamine utilization when compared to that of control. However, AP39 treatments caused dominant effects in glucose utilization (1.5 fold increase vs Dox at 24 hrs) and lactate production. Such high lactate production is one of the main reasons behind the modest in vitro protective ability of AP39 against Dox (FIG. 5), which is in contrast to the in vivo scenario (FIGS. 3 and 4). Thus, metabolomics and transcriptomic approaches led to better understanding of the protective function of AP39. All these novel data suggest that AP39 is highly protective against DIC and is a potent preclinical agent.

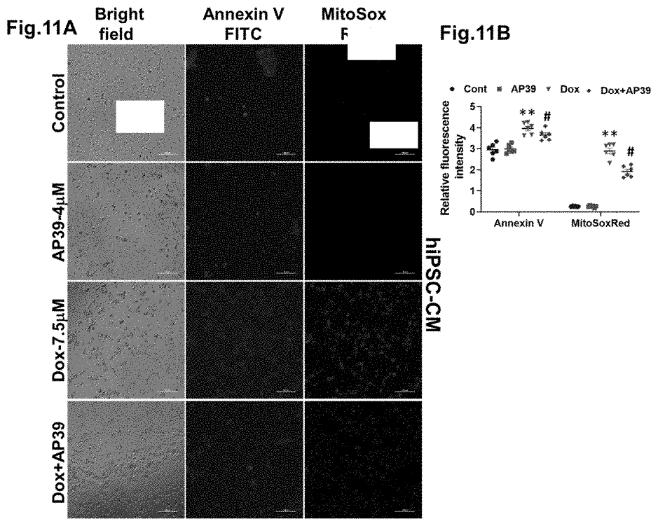

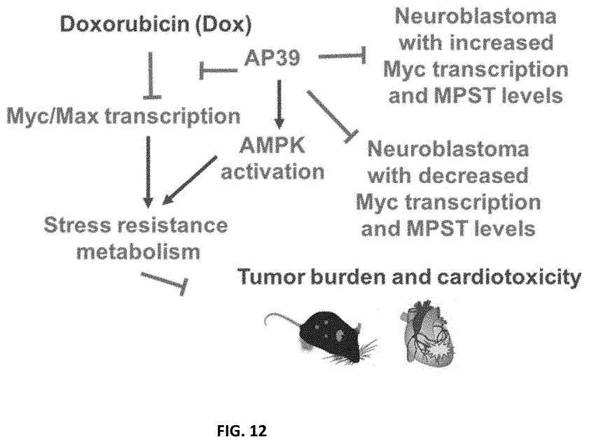

AP39 protects hiPSC-CMs from DIC. Commercially available hiPSC-CMs (a female, Caucasian donor) were treated with various doses of Dox and found that this particular genotype exhibited resistance to DIC at lower doses of Dox (2.5 μM, data not showed). A previous report suggested that certain patients exhibited enhanced Dox susceptibility at a lower dose than 2.5 μM Dox⁵. It is possible that this particular individual might belong to a Dox resistant group. A dose of 7.5 μM of Dox for a period of 48 hrs resulted in significant cell death (FIGS. 11A and B). Interestingly, a much lower dose of AP39 (4 μM, tenfold lower) attenuated DIC (FIGS. 11A and B) in hiPSC-CMs. Also, hiPSC-CMs exhibited lower tolerance to AP39 than H9C2 cells as a dose of 40 μM of AP39 resulted in greater susceptibility in the case of hiPSC-CMs. This could be because, unlike H9C2 cells, hiPSC-CMs formed syncytia and contract relentlessly (data not showed). Continuously contracting in vitro muscle is more susceptible to lactate toxicity as the lactate is not detoxified by liver and rapidly removed like in vivo situations⁵³. The instant data suggest that AP39 is a potent inducer of lactate production (FIG. 10D). Nonetheless, there was high correlation between level of apoptosis and levels of mitochondrial ROS, which were attenuated with AP39 treatment. These results suggested that hiPSC-CMs will represent a better predictive model for testing the clinical potential of AP39 against DIC, because of their relentless contraction ability, formation of syncytia, genetic diversity, ability to replicate AP39 mediated protection, and terminal differentiation (no cell division and no passaging while in culture, similar to in vivo cardiomyocytes).

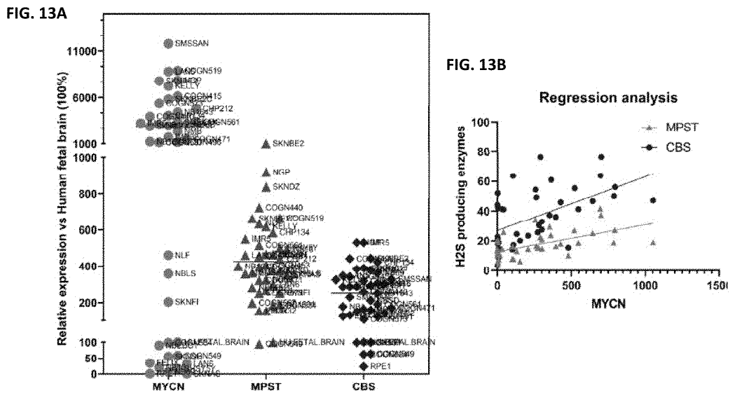

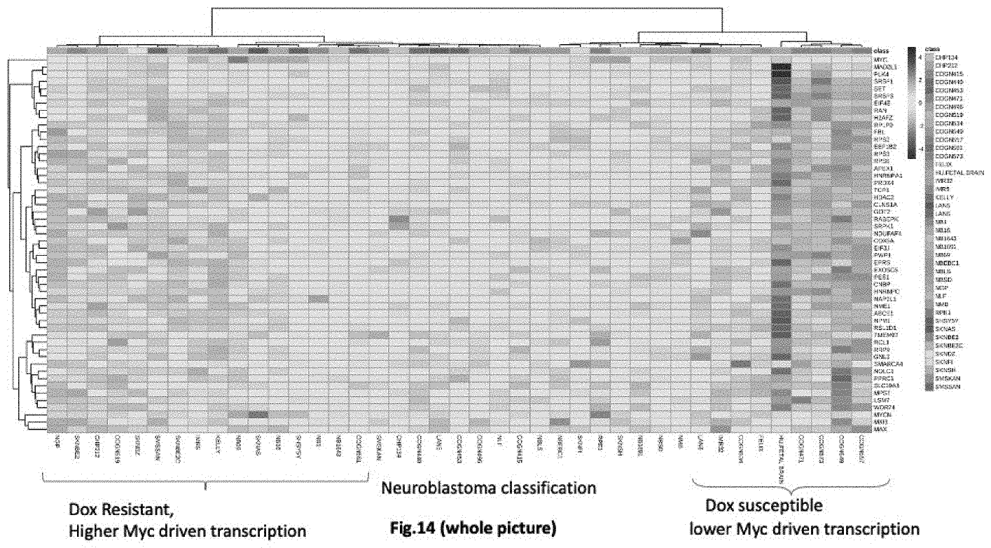

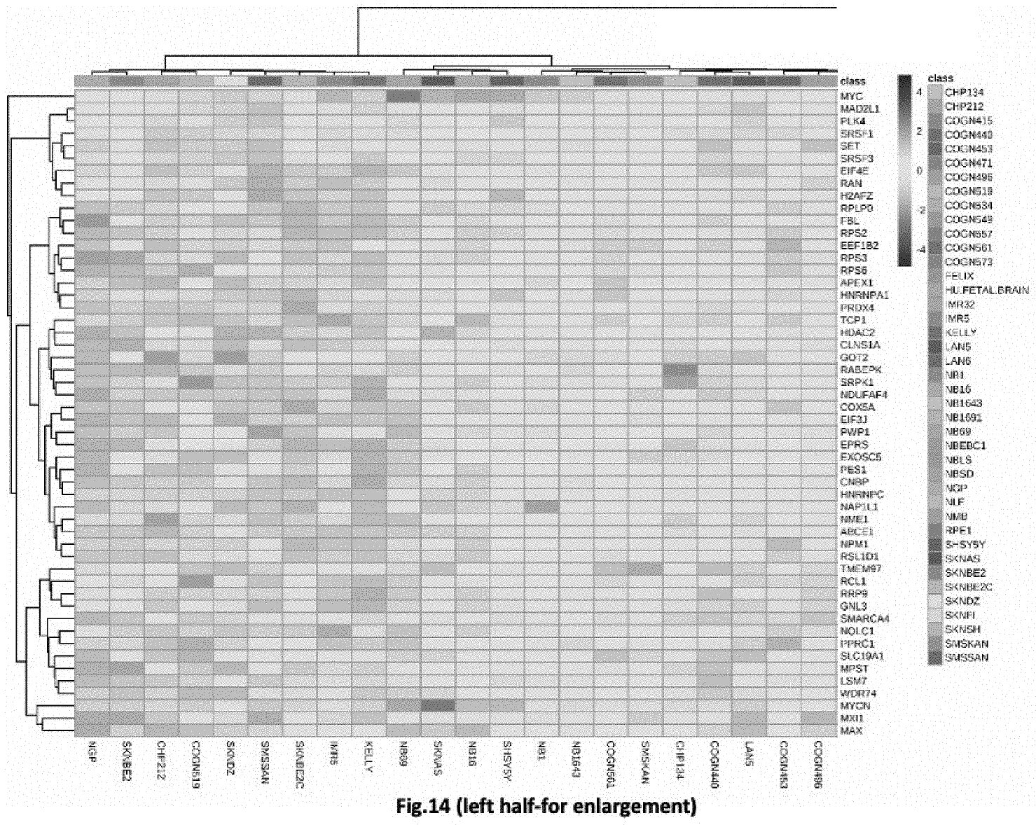

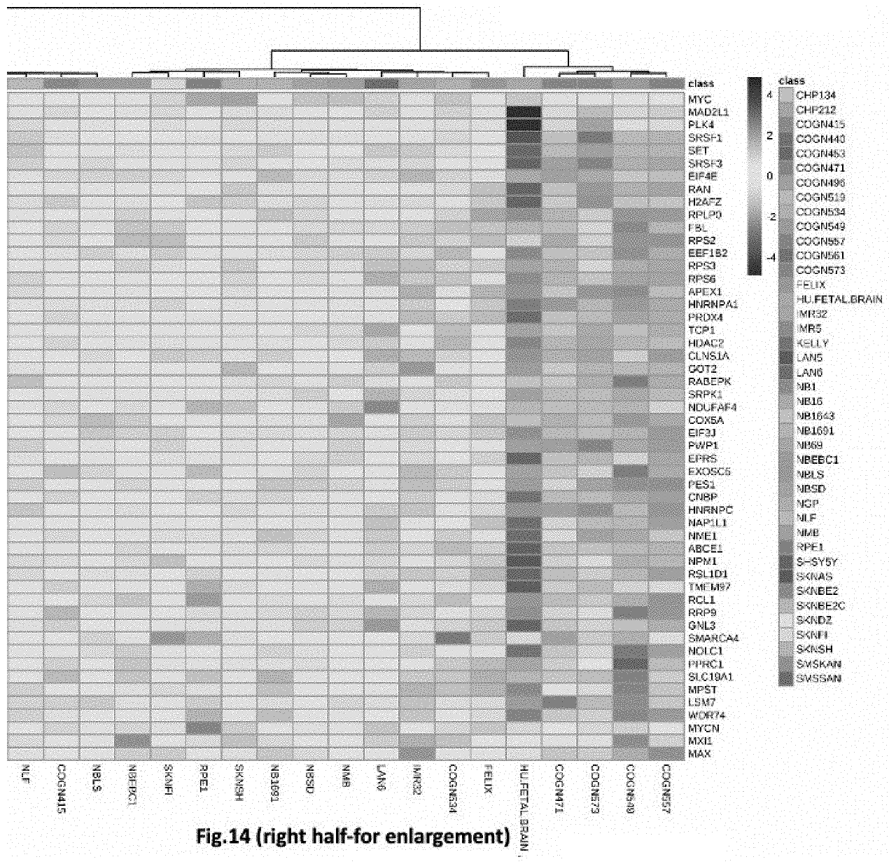

There is a significant positive correlation between the expression of Myc/Max gene transcription and H₂S producing enzymes: RNAseq data mining form the data deposited by a previous study that performed simultaneous RNAseq of 39 NB-human cell lines. 38 out of 39 human neuroblastoma cell lines upregulated MPST levels, a mitochondria-localized H₂S producer (FIG. 13A). Also, there is upregulation of another H₂S producing enzyme, CBS. There is a significant correlation between MYCN levels and H₂S producing enzymes: MPST and CBS (FIG. 13B). Highly co-regulated Myc dependent gene expressions and MPST levels were used to classify the neuroblastoma cell lines. Sometimes, MYCN amplification is not correlated with Myc-regulated transcription. For instance, SK-N-AS, NB-69, NB-16, SH-SY5Y cell lines do not show any MYCN amplification, but their Myc/Max dependent transcription is higher than the other group which shows MYCN amplification, such as COG-N-573, IMR-32 and COG-N-471(FIG. 14). We noted that such differences are because of the modulations in the other regulators of MYCN transcription such as Max (an obligate heterodimer for Myc transcription) and MXI1 (Max interactor 1). Currently, it is unknown whether Dox resistance is correlated with Myc transcription. However, the present classification of cell lines suggests that SK-N-BE(2) is more Dox resistant because of the higher Myc transcription than the IMR-32, which exhibits lower Myc transcription profile and Dox susceptibility (FIG. 14). We also noted that MPST levels seem to determine Dox resistance. SK-N-BE(2) cell line expresses much higher MPST levels than IMR-32, implying that H₂S levels could also confer Dox resistance.

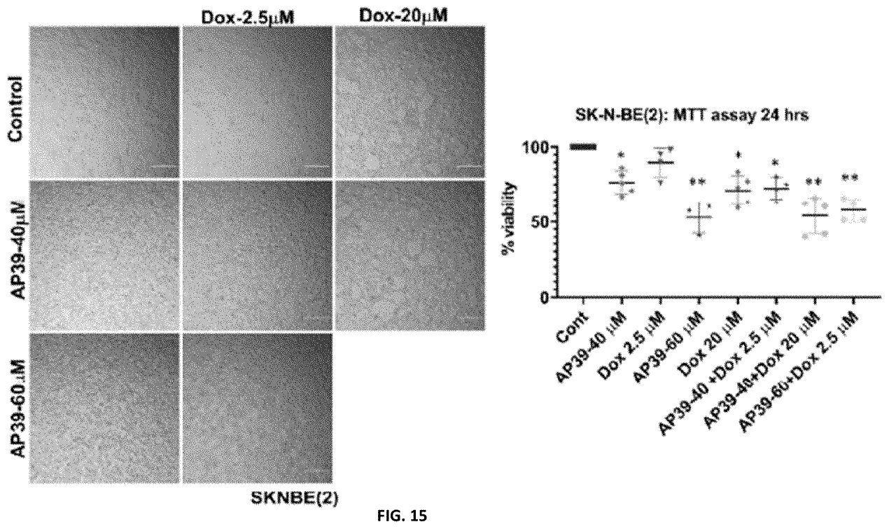

AP39 use reduces the Dox IC values to 8 fold lower in highly Dox resistant NB cell line SK-N-BE(2): Interestingly, a previous study derived two different cell lines from same patient before (SK-N-BE(1)) and after (SK-N-BE(2)) the treatment regimen of cyclophosphamide, doxorubicin, and vincristine. The cell line that was derived after the treatment (SK-N-BE(2)) is highly Dox resistance cell line compared to the SK-N-BE(1). The IC90 values for SK-N-BE(2) were greater than IC90 values for SK-N-BE(1) by 9230 times for doxorubicin. When AP39 was used along with the Dox, it reduced the effective Dox dose to 2.5 μM, nearly 8-fold lower than its original IC70 value (20 μM, FIG. 15). The results suggested that AP39 co-treatment is an effective therapeutic strategy, which will reduce Dox effective dose and thereby Dox toxic effects.

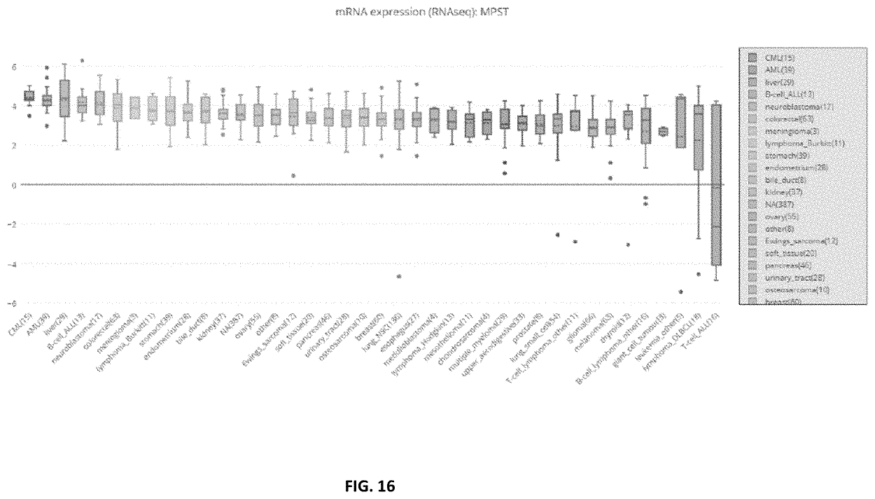

FIG. 16 shows the mRNA expression profile of MPST. MPST expression correlates with doxorubicin resistance as alluded to herein above. Also, higher MPST levels make the case for AP39 as an anti-cancer agent. Nearly all the cancer cells upregulate the three-key endogenous H₂S producing enzymes. The MPST is especially elevated nearly 2- to 4-fold (Cancer Cell Line Encyclopedia, Broad Institute) in 98% of the cancer cell lines belonging to 40 different types of cancers. Thus, cancer cells can be made prone to H₂S poisoning through external H₂S supplementation using H₂S donors, as they already elevate their H₂S levels more than normal tissues (for instance heart). Mitochondria targeting H₂S donor, AP39, was studied in different contexts, including as a cardioprotectant before, but it was neither tested against DIC nor are its effects in the heart completely known. Additionally, AP39 anti-cancer potential and its impact on Dox anticancer efficacy are not evaluated.

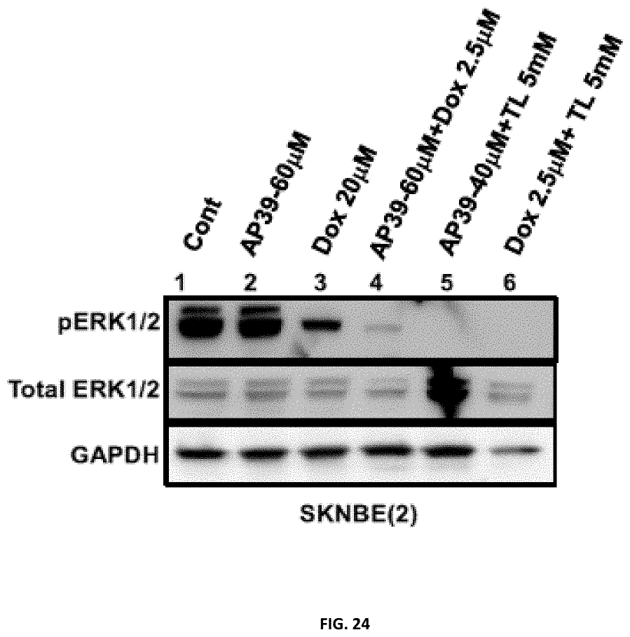

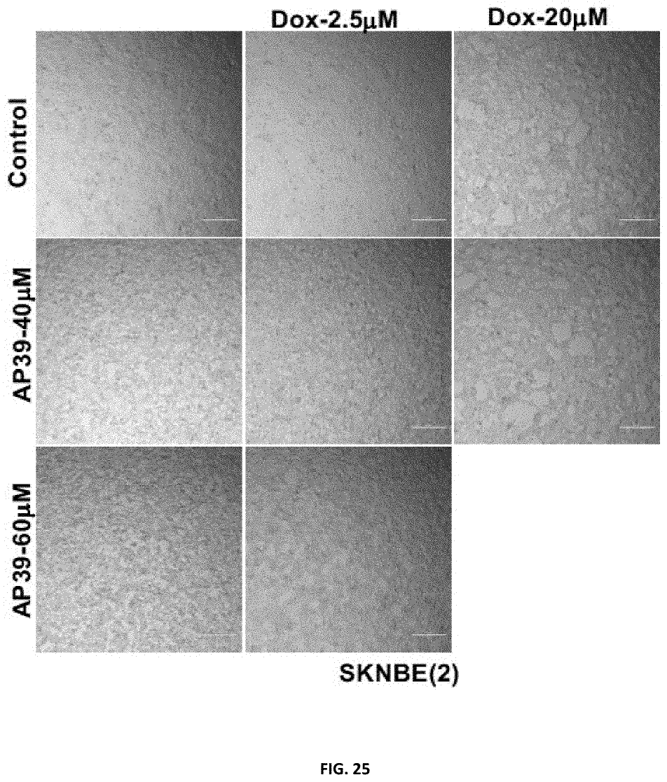

Methods for cell viability: Cells were plated in 96 wells/6 wells cell culture plates. After 24 hrs of plating, the treatments (added in the growth medium) were conducted. The following assays were performed after 24 hrs of the treatments (day 2). (FIGS. 15A, 17-20, 24-27)

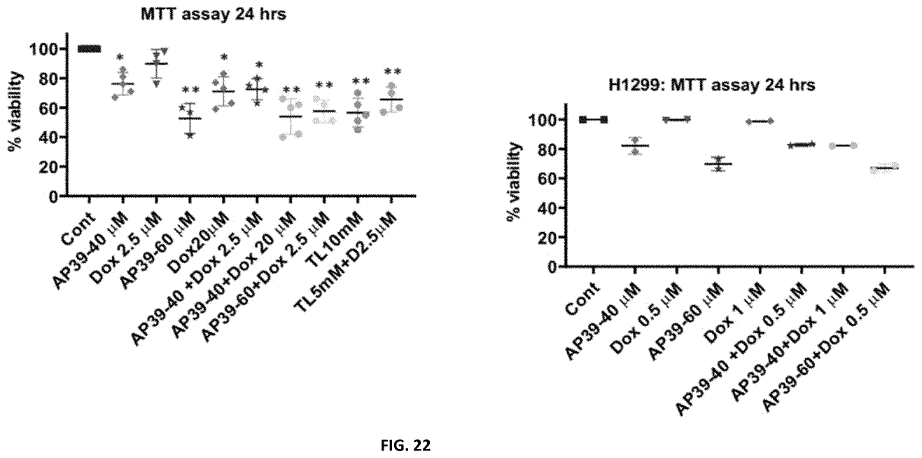

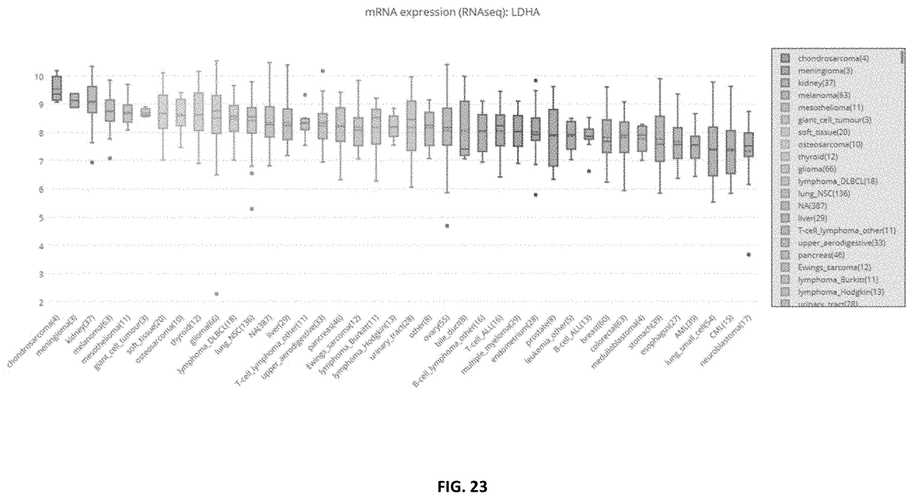

MTT assay methods. For the MTT assay, 100 μk of 5 mg/ml MTT-tetrazolium salts in PBS was added to each well containing the growth medium. After 3 hours of incubation, the medium was removed and the formazan crystals were dissolved by adding DMSO. The absorption of the formazan solution was measured using a spectrophotometer at a wavelength of 570 nm. FIGS. 15B, 21-22.

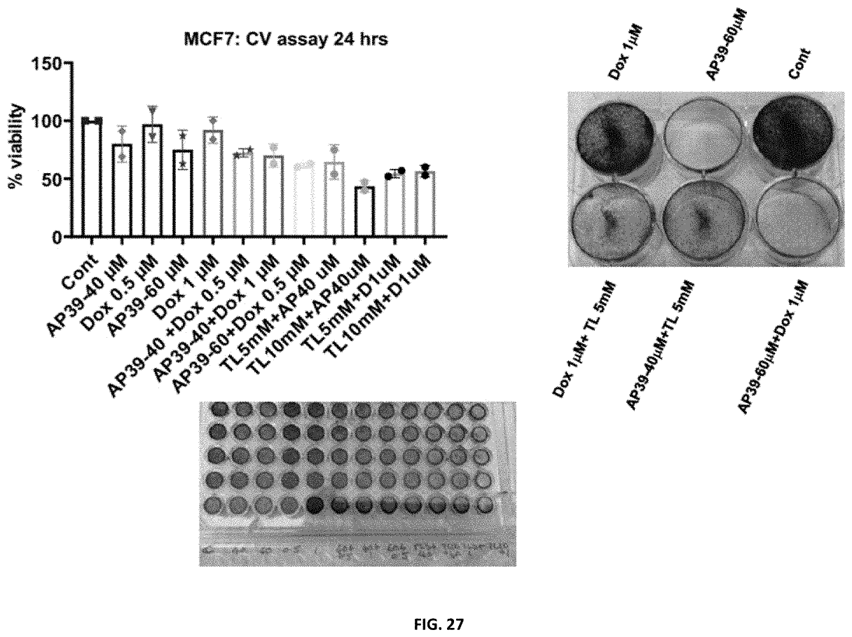

Crystal Violet (CV) assay. For the CV Staining assay, cells were stained with 0.5% crystal violet in 30% ethanol for 10 minutes at room temperature after removal of the medium. The cells were lysed in a 1% SDS (sodium dodecyl sulfate) solution. The absorbance of the solution was measured using a spectrophotometer at a wavelength of 595 nm. FIG. 27.

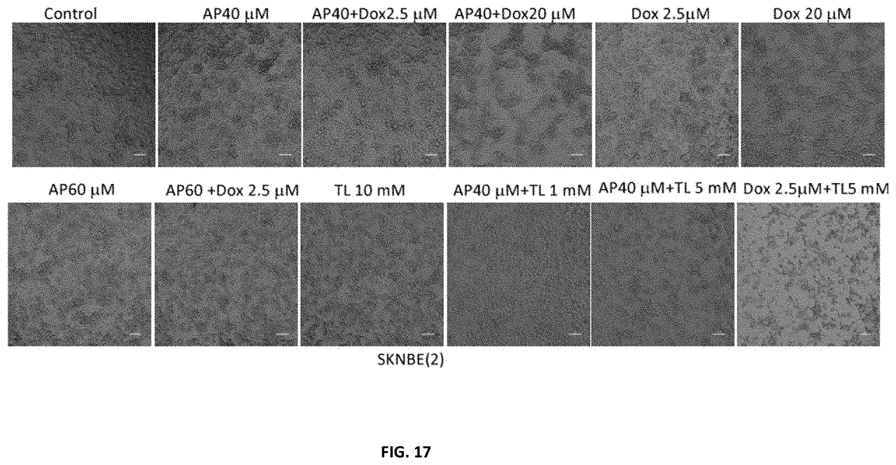

The SK-N-BE(2) neuroblastoma cell line was exposed to control, AP39 (AP) at 40 or 60 μM, Doxorubicin at 2.5 or 20 μM, thiolactate at 10 mM, AP39 and Doxorubicin, AP39 and thiolactate, or Doxorubicin and Thiolactate. Both AP39 and thiolactate alone were able to kill SK-N-BE(2) cells. AP39 and thiolactate were also able to increase the potency of low doses of Doxorubicin. FIGS. 17, 21, 24-25.

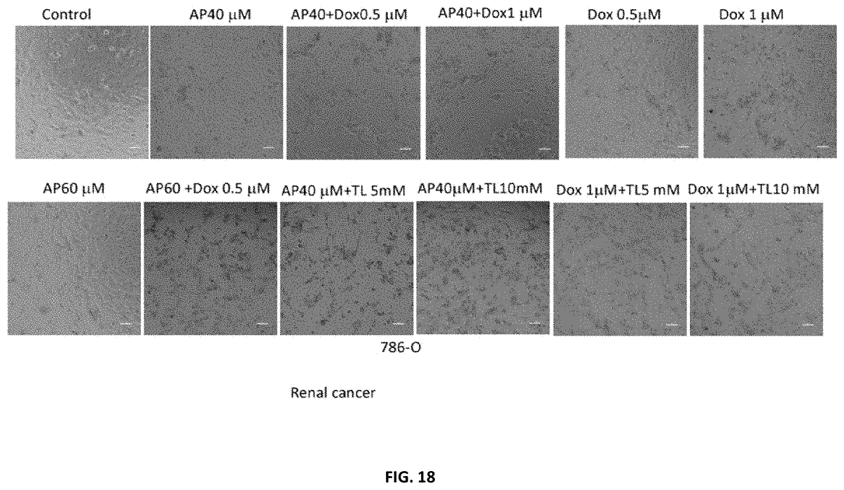

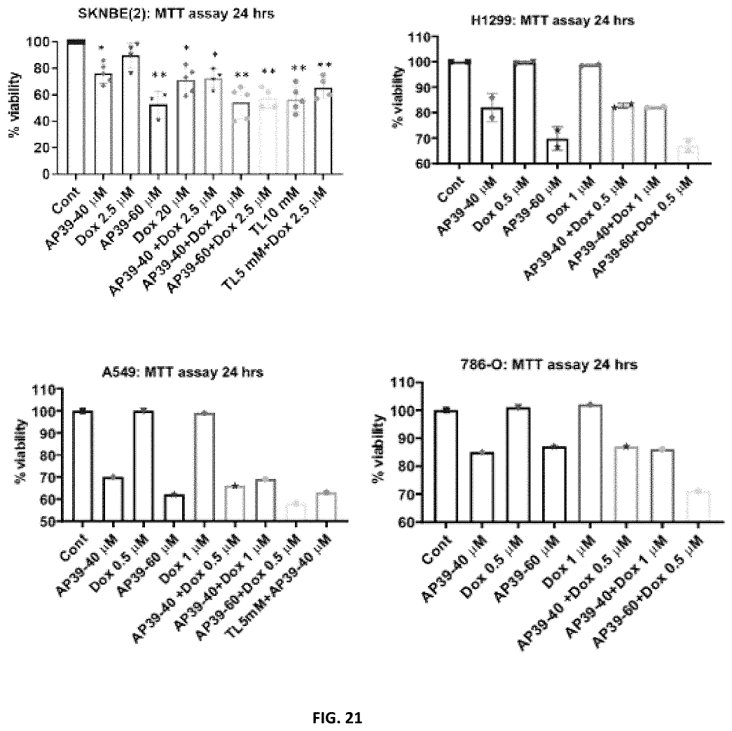

786-O cells renal cancer cells were exposed to control, AP39 (AP) at 40 or 60 μM, Doxorubicin at 2.5 or 20 μM, thiolactate at 10 mM, AP39 and Doxorubicin, AP39 and thiolactate, or Doxorubicin and Thiolactate. Both AP39 and thiolactate alone were able to kill renal cancer cells. AP39 and thiolactate were also able to increase the potency of low doses of Doxorubicin. FIGS. 18 and 21.

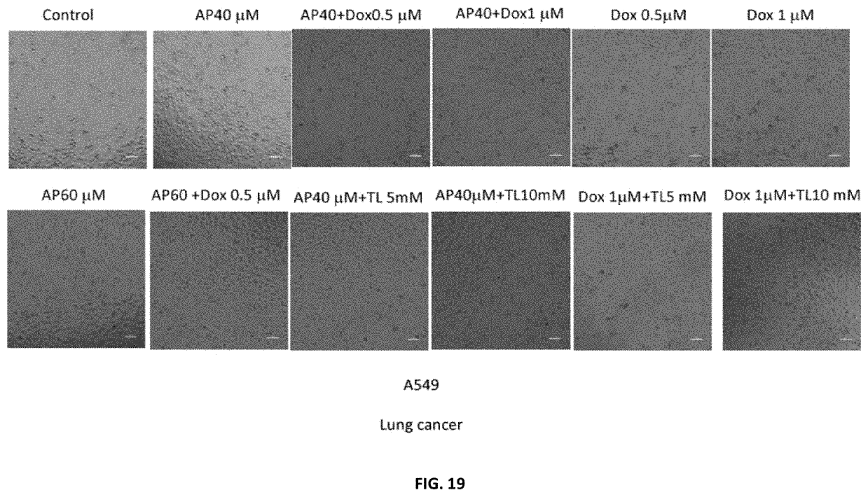

A549 lung cancer cells were exposed to control, AP39 (AP) at 40 or 60 μM, Doxorubicin at 2.5 or 20 μM, thiolactate at 10 mM, AP39 and Doxorubicin, AP39 and thiolactate, or Doxorubicin and Thiolactate. Both AP39 and thiolactate alone were able to kill lung cancer cells. AP39 and thiolactate were also able to increase the potency of low doses of Doxorubicin. FIGS. 19 and 21.

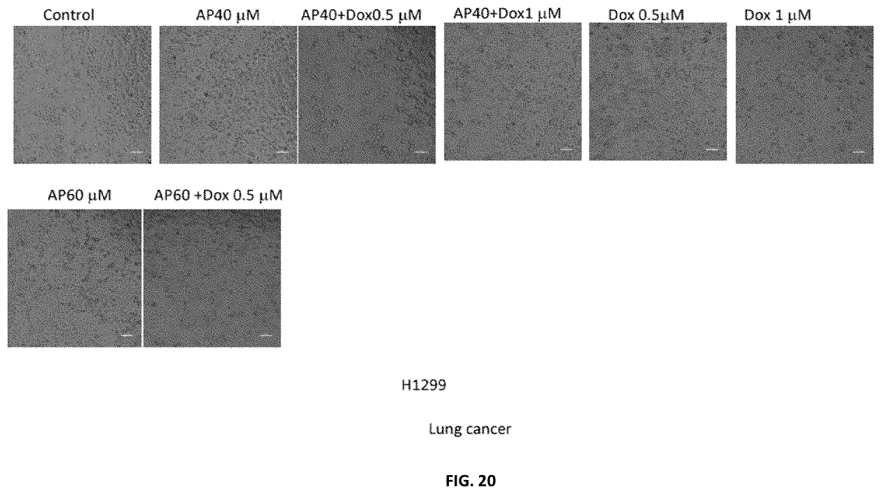

H1299 lung cancer cells were exposed to control, AP39 (AP) at 40 or 60 μM, Doxorubicin at 2.5 or 20 μM, thiolactate at 10 mM, AP39 and Doxorubicin, AP39 and thiolactate, or Doxorubicin and Thiolactate. Both AP39 and thiolactate alone were able to kill lung cancer cells. AP39 and thiolactate were also able to increase the potency of low doses of Doxorubicin. FIGS. 20-22.

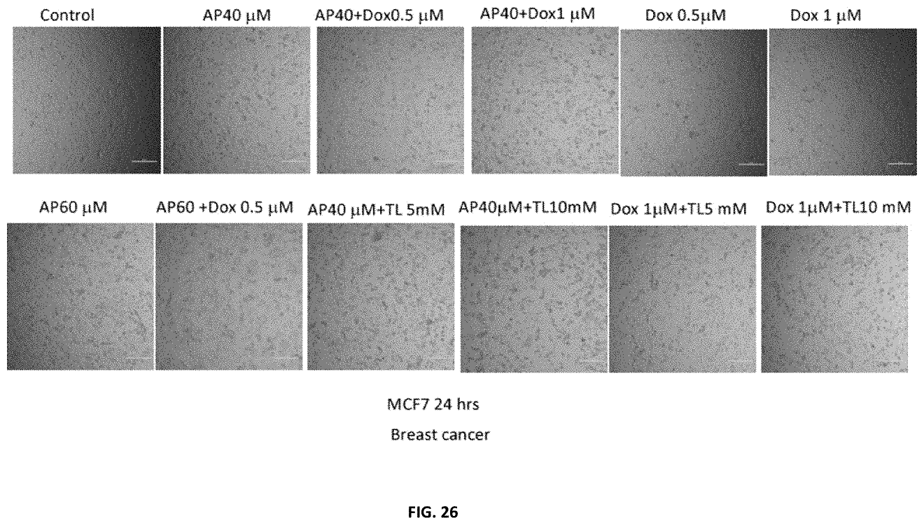

MCF7 breast cancer cells were exposed to control, AP39 (AP) at 40 or 60 μM, Doxorubicin at 2.5 or 20 μM, thiolactate at 10 mM, AP39 and Doxorubicin, AP39 and thiolactate, or Doxorubicin and Thiolactate. Both AP39 and thiolactate alone were able to kill breast cancer cells. AP39 and thiolactate were also able to increase the potency of low doses of Doxorubicin. FIGS. 26-27.

Tables 1-16 show a list of genes and their expression level in 39 distinct neuroblastoma cells lines. These genes are also represented in FIG. 13A and FIG. 13B.

TABLE 1
Gene expression in neuroblastoma cell lines

| | Cell Line | | | | | | | |
|---|---|---|---|---|---|---|---|---|
| Gene | CHP134 | CHP212 | COGN415 | COGN440 | COGN453 | COGN471 | COGN496 | COGN519 |
| MYCN | 361.261 | 428.219 | 547.763 | 353.204 | 262.398 | 130.549 | 103.856 | 794.052 |
| MXI1 | 5.713 | 3.744 | 6.468 | 8.590 | 7.457 | 5.394 | 2.928 | 8.130 |
| MPST | 23.862 | 18.022 | 18.623 | 29.471 | 16.521 | 13.142 | 16.106 | 27.141 |
| MAX | 7.309 | 10.403 | 6.806 | 8.524 | 6.700 | 4.748 | 5.167 | 9.748 |
| SMARCA4 | 30.009 | 23.912 | 23.991 | 33.419 | 14.933 | 8.531 | 33.617 | 32.056 |
| SET | 120.677 | 177.164 | 124.031 | 182.296 | 113.963 | 54.715 | 180.816 | 160.196 |
| RAN | 100.416 | 146.405 | 131.536 | 124.957 | 119.591 | 52.483 | 156.808 | 137.615 |
| NPM1 | 328.709 | 573.200 | 508.468 | 450.145 | 934.236 | 143.702 | 334.676 | 608.253 |
| FBL | 39.306 | 59.378 | 55.573 | 17.099 | 44.674 | 11.882 | 39.649 | 55.742 |
| NAP1L1 | 64.607 | 79.063 | 62.513 | 48.502 | 71.087 | 34.345 | 65.018 | 70.666 |
| SRSF1 | 71.577 | 83.431 | 69.932 | 73.005 | 73.580 | 31.669 | 58.006 | 80.199 |
| TCP1 | 43.505 | 116.250 | 84.019 | 102.978 | 37.206 | 33.506 | 51.770 | 134.756 |
| PWP1 | 16.286 | 24.016 | 15.898 | 21.144 | 12.372 | 6.169 | 11.792 | 17.440 |
| APEX1 | 41.781 | 87.304 | 51.357 | 48.499 | 54.811 | 22.284 | 34.372 | 52.860 |
| NME1 | 73.456 | 138.645 | 64.895 | 47.910 | 47.734 | 23.719 | 51.020 | 68.399 |
| EEF1B2 | 177.484 | 316.856 | 227.287 | 264.466 | 351.645 | 91.332 | 147.219 | 248.926 |
| RPS2 | 669.311 | 1071.991 | 953.667 | 449.834 | 1142.570 | 164.547 | 554.467 | 633.519 |
| RPS3 | 141.019 | 219.089 | 183.733 | 136.633 | 245.011 | 45.049 | 90.963 | 200.581 |
| SRSF3 | 59.452 | 73.303 | 60.534 | 73.500 | 53.600 | 20.722 | 55.567 | 75.810 |
| GOT2 | 38.062 | 58.345 | 30.089 | 38.831 | 21.395 | 16.089 | 24.123 | 34.883 |
| COX5A | 46.291 | 53.384 | 63.128 | 42.288 | 75.592 | 15.064 | 44.642 | 40.390 |
| HNRNPA1 | 8.200 | 11.697 | 11.609 | 10.954 | 12.447 | 3.225 | 10.986 | 12.044 |
| EIF3J | 12.187 | 27.562 | 16.426 | 16.741 | 15.844 | 6.151 | 13.701 | 16.264 |
| HDAC2 | 16.497 | 17.432 | 22.045 | 14.650 | 19.515 | 6.683 | 16.122 | 18.901 |
| CNBP | 52.337 | 67.753 | 65.542 | 42.791 | 49.832 | 22.448 | 56.831 | 69.570 |
| NOLC1 | 34.499 | 49.614 | 30.409 | 32.811 | 17.403 | 15.254 | 20.432 | 44.393 |
| PRDX4 | 33.683 | 90.210 | 75.099 | 72.861 | 49.692 | 18.638 | 59.676 | 86.200 |
| EIF4E | 8.786 | 12.215 | 10.111 | 11.241 | 8.764 | 3.356 | 7.871 | 10.443 |
| RPLP0 | 781.986 | 1025.132 | 779.648 | 540.764 | 1090.600 | 261.405 | 500.096 | 632.465 |
| ABCE1 | 31.622 | 49.974 | 27.810 | 27.127 | 21.807 | 10.873 | 19.202 | 36.381 |
| LSM7 | 54.340 | 71.131 | 67.678 | 83.515 | 56.046 | 10.557 | 43.420 | 66.028 |
| CLNS1A | 55.701 | 57.043 | 40.475 | 46.374 | 49.521 | 18.392 | 30.985 | 56.695 |
| RSL1D1 | 17.945 | 31.668 | 17.229 | 18.476 | 27.207 | 8.855 | 14.202 | 24.172 |
| HNRNPC | 119.605 | 138.719 | 100.563 | 115.097 | 109.197 | 39.659 | 113.366 | 137.274 |
| H2AFZ | 159.559 | 300.517 | 282.011 | 221.625 | 210.710 | 86.346 | 227.206 | 281.630 |
| EPRS | 71.602 | 50.962 | 34.761 | 54.272 | 41.996 | 15.318 | 29.725 | 32.557 |
| SRPK1 | 47.399 | 26.921 | 25.403 | 31.133 | 21.592 | 11.183 | 19.345 | 52.945 |
| MAD2L1 | 46.927 | 53.382 | 58.563 | 58.709 | 38.594 | 19.774 | 49.770 | 59.489 |
| RPS6 | 659.925 | 1062.872 | 453.430 | 758.988 | 1087.584 | 213.687 | 415.872 | 1530.287 |
| RRP9 | 9.400 | 20.728 | 28.102 | 25.359 | 14.790 | 7.057 | 19.893 | 19.670 |
| GNL3 | 25.921 | 67.787 | 62.480 | 58.830 | 47.470 | 16.707 | 58.095 | 39.896 |
| SLC19A1 | 6.542 | 8.420 | 9.574 | 8.562 | 5.622 | 3.081 | 5.608 | 10.116 |
| TMEM97 | 32.947 | 21.800 | 46.014 | 57.352 | 24.752 | 11.998 | 39.522 | 51.039 |
| PES1 | 19.689 | 34.341 | 22.342 | 24.269 | 18.992 | 11.807 | 20.826 | 33.842 |
| EXOSC5 | 22.102 | 20.743 | 31.312 | 9.148 | 16.082 | 6.534 | 18.094 | 30.362 |
| NDUFAF4 | 7.075 | 8.890 | 7.616 | 8.643 | 5.494 | 2.816 | 6.751 | 8.893 |
| RABEPK | 29.649 | 18.115 | 10.711 | 12.203 | 7.488 | 4.436 | 8.744 | 14.571 |
| WDR74 | 24.041 | 26.924 | 23.551 | 29.809 | 19.720 | 10.560 | 16.631 | 35.286 |
| PLK4 | 6.686 | 12.025 | 9.934 | 9.213 | 8.640 | 4.146 | 9.571 | 10.671 |
| RCL1 | 8.132 | 10.222 | 7.272 | 9.914 | 11.189 | 4.026 | 11.674 | 20.763 |
| PPRC1 | 7.232 | 12.136 | 10.183 | 8.436 | 2.659 | 3.751 | 6.558 | 14.800 |
| MYC | 0.181 | 0.051 | 0.197 | 0.071 | 0.112 | 0.089 | 0.375 | 0.031 |

TABLE 2
Gene Expression in Neuroblastoma cell line

| | Cell Line | | | | | | | |
|---|---|---|---|---|---|---|---|---|
| Gene | COGN534 | COGN549 | COGN557 | COGN561 | COGN573 | FELIX | HU.FETAL.BRAIN | IMR32 |
| MYCN | 8.810 | 5.183 | 101.220 | 301.107 | 479.777 | 3.104 | 8.937 | 152.787 |
| MXI1 | 3.406 | 1.912 | 4.055 | 7.686 | 5.135 | 6.769 | 7.589 | 2.713 |
| MPST | 7.556 | 3.863 | 8.060 | 21.038 | 10.277 | 6.282 | 4.076 | 6.289 |
| MAX | 7.626 | 4.036 | 3.444 | 7.331 | 4.978 | 7.678 | 5.642 | 3.517 |
| SMARCA4 | 6.173 | 9.694 | 15.906 | 27.326 | 12.911 | 13.229 | 14.703 | 15.078 |
| SET | 76.530 | 51.676 | 50.423 | 110.732 | 42.689 | 101.323 | 24.949 | 63.172 |
| RAN | 71.193 | 42.740 | 38.232 | 104.664 | 33.848 | 51.637 | 18.450 | 78.151 |
| NPM1 | 167.523 | 79.500 | 89.596 | 457.301 | 176.673 | 132.376 | 16.269 | 238.330 |
| FBL | 12.871 | 6.075 | 10.477 | 49.533 | 18.306 | 13.711 | 10.795 | 16.002 |
| NAP1L1 | 52.466 | 23.949 | 18.156 | 65.716 | 25.047 | 26.867 | 10.402 | 44.521 |
| SRSF1 | 39.536 | 29.719 | 27.952 | 69.178 | 17.731 | 56.601 | 11.392 | 45.149 |
| TCP1 | 30.188 | 30.645 | 25.145 | 38.405 | 23.575 | 54.465 | 22.679 | 52.086 |
| PWP1 | 14.889 | 6.677 | 6.025 | 14.408 | 4.987 | 12.552 | 6.254 | 10.893 |
| APEX1 | 29.789 | 12.301 | 19.745 | 70.716 | 13.554 | 17.773 | 13.620 | 17.288 |
| NME1 | 19.888 | 11.043 | 17.975 | 51.556 | 10.266 | 26.557 | 4.826 | 34.053 |
| EEF1B2 | 85.287 | 58.262 | 74.502 | 269.860 | 95.663 | 152.748 | 54.730 | 118.776 |
| RPS2 | 324.570 | 125.369 | 127.213 | 920.901 | 312.011 | 223.876 | 313.180 | 280.921 |
| RPS3 | 73.532 | 41.663 | 38.281 | 223.115 | 76.257 | 66.797 | 37.558 | 52.836 |
| SRSF3 | 43.234 | 23.492 | 22.046 | 57.933 | 16.463 | 53.636 | 11.626 | 36.120 |
| GOT2 | 21.236 | 13.728 | 12.898 | 19.395 | 12.957 | 31.337 | 10.827 | 10.508 |
| COX5A | 27.993 | 11.621 | 13.712 | 37.057 | 17.420 | 20.130 | 18.247 | 22.799 |
| HNRNPA1 | 8.569 | 3.202 | 3.728 | 14.639 | 4.152 | 4.970 | 2.453 | 5.221 |
| EIF3J | 8.932 | 5.789 | 4.492 | 12.500 | 6.506 | 7.528 | 4.122 | 7.883 |
| HDAC2 | 6.849 | 6.678 | 7.282 | 15.048 | 5.820 | 15.225 | 4.313 | 12.523 |
| CNBP | 36.758 | 18.959 | 17.264 | 44.775 | 22.385 | 32.251 | 11.210 | 27.148 |
| NOLC1 | 24.010 | 6.727 | 9.724 | 30.491 | 18.924 | 22.242 | 6.085 | 17.572 |
| PRDX4 | 22.201 | 12.696 | 14.055 | 55.307 | 16.996 | 16.915 | 5.837 | 17.252 |
| EIF4E | 5.316 | 4.719 | 4.269 | 7.500 | 3.640 | 5.783 | 3.606 | 3.985 |
| RPLP0 | 405.092 | 192.402 | 201.090 | 904.914 | 371.373 | 231.213 | 180.096 | 503.448 |
| ABCE1 | 9.168 | 8.838 | 8.358 | 22.769 | 10.042 | 11.982 | 2.968 | 13.558 |
| LSM7 | 29.601 | 12.082 | 22.912 | 39.968 | 19.402 | 28.653 | 14.123 | 21.395 |
| CLNS1A | 27.223 | 25.322 | 15.404 | 41.430 | 16.254 | 39.127 | 14.169 | 20.097 |
| RSL1D1 | 11.183 | 8.112 | 6.519 | 24.212 | 10.782 | 8.272 | 3.692 | 9.994 |
| HNRNPC | 76.693 | 49.274 | 36.414 | 105.270 | 34.934 | 66.890 | 36.212 | 55.039 |
| H2AFZ | 124.242 | 79.609 | 81.234 | 174.340 | 46.461 | 85.329 | 25.376 | 106.930 |
| EPRS | 28.051 | 20.233 | 10.813 | 28.886 | 13.060 | 19.648 | 4.518 | 21.757 |
| SRPK1 | 16.312 | 9.504 | 10.911 | 16.672 | 11.392 | 20.550 | 8.787 | 14.852 |
| MAD2L1 | 16.850 | 18.750 | 15.589 | 31.595 | 11.307 | 22.110 | 0.780 | 26.521 |
| RPS6 | 275.273 | 185.470 | 182.728 | 890.036 | 278.583 | 396.643 | 140.580 | 297.448 |
| RRP9 | 8.392 | 2.399 | 3.103 | 15.660 | 7.911 | 7.366 | 2.953 | 7.285 |
| GNL3 | 20.911 | 15.351 | 12.421 | 50.328 | 21.111 | 18.289 | 4.892 | 24.927 |
| SLC19A1 | 3.105 | 1.653 | 3.322 | 8.728 | 2.797 | 2.458 | 2.608 | 3.178 |
| TMEM97 | 15.853 | 13.700 | 15.283 | 58.357 | 9.419 | 14.608 | 2.280 | 19.816 |
| PES1 | 10.856 | 5.609 | 5.581 | 18.437 | 6.666 | 14.980 | 6.336 | 9.081 |
| EXOSC5 | 7.250 | 2.750 | 4.626 | 13.185 | 8.096 | 13.998 | 4.238 | 5.770 |
| NDUFAF4 | 4.193 | 2.457 | 3.594 | 5.460 | 2.714 | 4.204 | 2.628 | 4.633 |
| RABEPK | 4.548 | 1.926 | 3.176 | 9.208 | 3.290 | 6.417 | 4.183 | 8.915 |
| WDR74 | 20.001 | 5.818 | 6.771 | 20.560 | 11.610 | 12.794 | 5.419 | 10.190 |
| PLK4 | 4.599 | 4.541 | 4.423 | 6.838 | 1.898 | 4.715 | 0.272 | 5.397 |
| RCL1 | 4.069 | 3.446 | 2.726 | 11.234 | 4.609 | 6.041 | 2.779 | 5.598 |
| PPRC1 | 3.551 | 1.171 | 2.997 | 7.863 | 5.954 | 2.972 | 2.364 | 5.446 |
| MYC | 0.012 | 0.726 | 0.190 | 0.059 | 0.096 | 0.809 | 3.286 | 0.061 |

TABLE 3
Gene expression in neuroblastoma cell lines

| | Cell Line | | | | | | | |
|---|---|---|---|---|---|---|---|---|
| Gene | IMR5 | KELLY | LAN5 | LAN6 | NB1 | NB16 | NB1643 | NB1691 |
| MYCN | 289.376 | 644.877 | 786.076 | 2.771 | 273.312 | 1.773 | 395.478 | 108.942 |
| MXI1 | 9.829 | 12.417 | 14.203 | 3.931 | 4.723 | 5.313 | 7.050 | 9.804 |
| MPST | 22.435 | 25.194 | 18.790 | 12.192 | 15.291 | 20.075 | 16.288 | 7.766 |
| MAX | 7.116 | 7.375 | 10.090 | 6.883 | 7.520 | 4.398 | 5.782 | 4.833 |
| SMARCA4 | 24.281 | 28.621 | 29.178 | 19.547 | 25.872 | 19.758 | 23.128 | 19.434 |
| SET | 138.895 | 130.513 | 134.025 | 63.794 | 86.567 | 135.728 | 78.655 | 66.152 |
| RAN | 202.757 | 171.237 | 120.032 | 113.312 | 103.035 | 131.296 | 81.214 | 75.750 |
| NPM1 | 798.954 | 858.626 | 205.326 | 163.451 | 269.024 | 674.899 | 344.213 | 283.493 |
| FBL | 63.071 | 81.565 | 29.170 | 18.688 | 29.823 | 53.289 | 35.961 | 44.882 |
| NAP1L1 | 53.536 | 116.150 | 48.411 | 27.104 | 19.377 | 87.861 | 59.951 | 51.397 |
| SRSF1 | 73.127 | 70.990 | 73.551 | 49.209 | 49.215 | 52.011 | 67.205 | 45.043 |
| TCP1 | 161.813 | 116.858 | 71.357 | 23.796 | 55.310 | 137.966 | 50.952 | 67.903 |
| PWP1 | 19.223 | 18.936 | 15.642 | 13.408 | 19.701 | 20.508 | 13.009 | 17.512 |
| APEX1 | 57.070 | 73.264 | 51.480 | 29.863 | 57.939 | 50.207 | 46.086 | 38.386 |
| NME1 | 60.731 | 98.757 | 66.459 | 18.315 | 38.346 | 36.024 | 53.924 | 15.865 |
| EEF1B2 | 293.454 | 202.378 | 204.598 | 107.716 | 169.655 | 253.030 | 230.727 | 129.882 |
| RPS2 | 1388.890 | 1473.079 | 442.038 | 450.411 | 632.388 | 1073.400 | 778.175 | 633.882 |
| RPS3 | 211.758 | 296.795 | 104.097 | 57.131 | 108.832 | 226.228 | 162.933 | 131.120 |
| SRSF3 | 64.501 | 80.308 | 66.980 | 39.207 | 50.527 | 72.193 | 46.463 | 44.675 |
| GOT2 | 27.760 | 29.993 | 41.594 | 17.369 | 34.984 | 20.908 | 19.293 | 29.545 |
| COX5A | 62.134 | 77.767 | 35.845 | 39.190 | 59.953 | 70.198 | 42.743 | 27.792 |
| HNRNPA1 | 12.337 | 11.378 | 7.442 | 7.328 | 8.545 | 9.196 | 11.145 | 9.213 |
| EIF3J | 24.902 | 23.573 | 14.586 | 10.339 | 15.267 | 12.007 | 14.404 | 9.366 |
| HDAC2 | 20.967 | 26.737 | 16.895 | 8.855 | 11.834 | 21.784 | 17.909 | 19.943 |
| CNBP | 61.016 | 97.445 | 52.659 | 40.264 | 39.175 | 68.325 | 32.738 | 29.646 |
| NOLC1 | 69.055 | 39.070 | 37.901 | 23.849 | 37.701 | 45.368 | 34.006 | 18.068 |
| PRDX4 | 65.462 | 76.354 | 62.676 | 31.549 | 41.425 | 50.028 | 40.764 | 61.676 |
| EIF4E | 10.904 | 13.547 | 11.169 | 5.322 | 9.273 | 8.706 | 6.182 | 4.246 |
| RPLP0 | 1198.520 | 1281.980 | 602.731 | 588.309 | 469.627 | 962.850 | 731.842 | 1301.165 |
| ABCE1 | 43.890 | 41.760 | 30.447 | 11.900 | 22.272 | 35.889 | 21.347 | 13.528 |
| LSM7 | 57.444 | 60.639 | 40.852 | 29.235 | 57.274 | 46.034 | 31.504 | 29.487 |
| CLNS1A | 66.135 | 59.210 | 49.477 | 18.070 | 37.196 | 40.038 | 32.964 | 25.349 |
| RSL1D1 | 27.430 | 31.263 | 19.104 | 11.017 | 15.463 | 28.190 | 18.171 | 14.112 |
| HNRNPC | 159.326 | 164.688 | 109.139 | 59.790 | 78.901 | 136.915 | 78.280 | 79.461 |
| H2AFZ | 253.609 | 320.392 | 227.570 | 111.491 | 152.085 | 194.739 | 156.199 | 113.922 |
| EPRS | 80.830 | 98.908 | 26.632 | 33.793 | 47.492 | 43.920 | 44.941 | 42.195 |
| SRPK1 | 31.948 | 41.457 | 27.984 | 10.542 | 15.978 | 13.831 | 16.636 | 16.118 |
| MAD2L1 | 52.777 | 63.316 | 77.571 | 16.875 | 34.188 | 36.258 | 35.576 | 21.602 |
| RPS6 | 998.870 | 1146.248 | 562.849 | 208.909 | 553.788 | 658.861 | 571.272 | 727.538 |
| RRP9 | 22.666 | 30.884 | 13.149 | 4.592 | 10.683 | 20.592 | 9.485 | 7.518 |
| GNL3 | 79.642 | 88.163 | 60.336 | 9.748 | 42.126 | 60.193 | 39.119 | 25.077 |
| SLC19A1 | 6.122 | 5.369 | 9.145 | 5.015 | 5.946 | 5.580 | 6.145 | 2.499 |
| TMEM97 | 34.449 | 34.775 | 42.302 | 7.832 | 28.257 | 21.346 | 33.486 | 24.589 |
| PES1 | 21.142 | 42.574 | 22.832 | 14.168 | 17.484 | 29.885 | 19.837 | 9.790 |
| EXOSC5 | 21.173 | 35.409 | 16.528 | 11.884 | 13.631 | 16.300 | 10.634 | 11.092 |
| NDUFAF4 | 7.217 | 11.647 | 7.317 | 1.713 | 4.747 | 7.054 | 4.423 | 6.528 |
| RABEPK | 12.796 | 12.046 | 8.328 | 5.761 | 9.319 | 10.878 | 5.531 | 5.302 |
| WDR74 | 20.315 | 29.982 | 25.173 | 16.885 | 18.694 | 20.459 | 14.843 | 9.561 |
| PLK4 | 10.545 | 9.354 | 12.452 | 5.744 | 7.734 | 9.020 | 8.053 | 4.821 |
| RCL1 | 13.220 | 15.221 | 11.885 | 8.352 | 7.967 | 6.135 | 8.808 | 5.786 |
| PPRC1 | 11.739 | 10.389 | 10.359 | 5.747 | 8.366 | 7.368 | 9.473 | 4.690 |
| MYC | 0.000 | 0.025 | 0.070 | 2.030 | 0.018 | 17.040 | 0.022 | 0.122 |

TABLE 4
Gene expression in neuroblastoma cell line

| | Cell Line | | | | | | | |
|---|---|---|---|---|---|---|---|---|
| Gene | NB69 | NBEBC1 | NBLS | NBSD | NGP | NLF | NMB | RPE1 |
| MYCN | 0.570 | 8.053 | 31.970 | 280.671 | 700.038 | 41.178 | 208.079 | 0.085 |
| MXI1 | 6.521 | 1.992 | 8.436 | 7.851 | 16.084 | 4.626 | 6.431 | 4.625 |
| MPST | 13.316 | 11.483 | 14.615 | 15.180 | 37.451 | 25.894 | 14.616 | 10.892 |
| MAX | 4.516 | 8.940 | 7.524 | 5.440 | 11.075 | 6.008 | 6.563 | 7.467 |
| SMARCA4 | 25.503 | 16.873 | 29.981 | 25.269 | 38.761 | 24.336 | 19.479 | 9.593 |
| SET | 126.079 | 136.868 | 108.545 | 115.980 | 159.194 | 174.647 | 98.419 | 96.177 |
| RAN | 134.374 | 128.530 | 129.690 | 83.661 | 135.119 | 124.207 | 84.081 | 122.168 |
| NPM1 | 520.328 | 172.879 | 290.033 | 174.317 | 633.101 | 536.492 | 210.718 | 342.794 |
| FBL | 59.380 | 14.099 | 42.532 | 19.114 | 128.548 | 35.819 | 18.353 | 28.950 |
| NAP1L1 | 69.348 | 47.646 | 49.118 | 44.236 | 119.605 | 66.924 | 57.160 | 64.678 |
| SRSF1 | 71.808 | 61.972 | 58.673 | 44.853 | 69.515 | 83.868 | 44.756 | 44.705 |
| TCP1 | 97.093 | 70.977 | 81.985 | 95.451 | 95.373 | 68.020 | 41.944 | 74.795 |
| PWP1 | 27.512 | 16.664 | 12.400 | 16.041 | 26.345 | 9.771 | 9.760 | 16.193 |
| APEX1 | 33.489 | 31.425 | 51.350 | 38.401 | 64.015 | 57.634 | 34.085 | 37.280 |
| NME1 | 91.254 | 29.329 | 39.512 | 22.743 | 61.198 | 51.470 | 28.882 | 24.668 |
| EEF1B2 | 208.373 | 131.733 | 271.322 | 116.874 | 302.958 | 185.460 | 112.177 | 147.368 |
| RPS2 | 1019.556 | 226.423 | 683.637 | 281.817 | 1560.447 | 590.705 | 426.039 | 770.216 |
| RPS3 | 187.741 | 71.989 | 154.110 | 83.618 | 450.528 | 124.086 | 85.620 | 121.718 |
| SRSF3 | 55.682 | 63.375 | 73.050 | 48.882 | 68.234 | 76.693 | 37.673 | 53.003 |
| GOT2 | 38.090 | 24.616 | 31.543 | 26.724 | 47.084 | 20.408 | 22.881 | 29.405 |
| COX5A | 75.156 | 21.845 | 87.663 | 27.973 | 80.267 | 39.209 | 13.302 | 34.231 |
| HNRNPA1 | 11.605 | 6.557 | 11.269 | 7.105 | 13.131 | 6.869 | 6.916 | 7.216 |
| EIF3J | 24.852 | 8.526 | 23.495 | 9.955 | 29.377 | 15.650 | 8.792 | 13.337 |
| HDAC2 | 16.985 | 11.832 | 18.035 | 17.503 | 33.074 | 16.778 | 10.380 | 12.498 |
| CNBP | 63.436 | 52.116 | 47.912 | 33.003 | 81.891 | 55.042 | 35.940 | 28.435 |
| NOLC1 | 53.676 | 16.640 | 40.268 | 20.845 | 37.439 | 27.843 | 34.312 | 26.127 |
| PRDX4 | 66.110 | 36.402 | 34.821 | 39.220 | 97.180 | 60.956 | 31.469 | 46.293 |
| EIF4E | 4.811 | 5.034 | 10.413 | 5.540 | 10.285 | 9.198 | 5.677 | 8.980 |
| RPLP0 | 836.921 | 395.440 | 816.989 | 420.175 | 1343.204 | 578.184 | 556.801 | 774.252 |
| ABCE1 | 39.084 | 13.530 | 21.452 | 15.096 | 39.391 | 27.804 | 16.931 | 27.624 |
| LSM7 | 56.694 | 31.284 | 72.155 | 37.361 | 85.033 | 64.894 | 27.083 | 24.766 |
| CLNS1A | 47.023 | 46.765 | 57.557 | 41.381 | 81.391 | 47.933 | 38.426 | 26.019 |
| RSL1D1 | 24.445 | 13.626 | 23.160 | 16.583 | 30.095 | 24.793 | 20.007 | 11.502 |
| HNRNPC | 82.854 | 122.780 | 117.717 | 71.388 | 136.953 | 152.445 | 75.643 | 91.783 |
| H2AFZ | 134.246 | 116.199 | 172.637 | 103.219 | 151.166 | 231.424 | 133.199 | 300.753 |
| EPRS | 65.644 | 34.239 | 29.209 | 21.656 | 97.886 | 25.137 | 18.994 | 26.697 |
| SRPK1 | 22.079 | 18.090 | 18.618 | 32.468 | 36.407 | 19.729 | 20.213 | 21.594 |
| MAD2L1 | 25.412 | 23.037 | 38.104 | 28.878 | 37.439 | 48.682 | 32.170 | 42.088 |
| RPS6 | 833.568 | 458.491 | 705.523 | 383.252 | 1494.047 | 977.577 | 498.226 | 661.175 |
| RRP9 | 21.456 | 15.819 | 12.911 | 6.769 | 16.686 | 15.628 | 7.699 | 7.670 |
| GNL3 | 58.937 | 40.853 | 41.276 | 19.567 | 30.549 | 50.108 | 20.833 | 25.810 |
| SLC19A1 | 7.542 | 3.053 | 4.917 | 6.301 | 9.593 | 5.201 | 5.139 | 2.936 |
| TMEM97 | 38.714 | 25.596 | 21.549 | 31.739 | 38.271 | 26.739 | 26.456 | 7.626 |
| PES1 | 31.823 | 28.206 | 23.052 | 10.571 | 42.033 | 30.740 | 11.787 | 18.276 |
| EXOSC5 | 26.016 | 11.567 | 22.966 | 8.499 | 34.984 | 17.533 | 12.371 | 5.873 |
| NDUFAF4 | 7.253 | 3.841 | 5.130 | 4.370 | 11.826 | 5.600 | 3.298 | 2.520 |
| RABEPK | 5.098 | 7.386 | 7.494 | 6.587 | 16.854 | 16.607 | 4.898 | 7.506 |
| WDR74 | 28.162 | 17.464 | 21.796 | 17.716 | 27.897 | 27.978 | 15.692 | 8.365 |
| PLK4 | 4.445 | 6.631 | 10.431 | 5.713 | 8.999 | 11.918 | 6.018 | 9.744 |
| RCL1 | 12.737 | 13.859 | 11.250 | 6.609 | 9.513 | 7.593 | 5.811 | 2.926 |
| PPRC1 | 11.450 | 3.420 | 7.492 | 4.474 | 9.859 | 7.438 | 5.765 | 5.290 |
| MYC | 165.137 | 0.115 | 0.191 | 0.011 | 0.118 | 0.134 | 0.010 | 18.582 |

TABLE 5
Gene expression in neuroblastoma cell lines

| | Cell Line | | | | | | | |
|---|---|---|---|---|---|---|---|---|
| Gene | SHSY5Y | SKNAS | SKNBE2 | SKNBE2C | SKNDZ | SKNFI | SKNSH | SMSKAN |
| MYCN | 1.451 | 0.046 | 695.209 | 520.553 | 256.233 | 18.155 | 4.962 | 301.818 |
| MXI1 | 5.327 | 5.760 | 16.499 | 7.278 | 9.237 | 7.845 | 12.631 | 11.047 |
| MPST | 20.426 | 14.359 | 41.759 | 27.022 | 34.107 | 10.317 | 18.931 | 14.775 |
| MAX | 7.821 | 9.033 | 8.825 | 6.165 | 5.062 | 9.029 | 7.981 | 6.933 |
| SMARCA4 | 26.997 | 16.545 | 33.665 | 25.484 | 29.151 | 7.832 | 16.265 | 25.926 |
| SET | 127.018 | 89.213 | 133.778 | 120.018 | 161.399 | 108.930 | 77.062 | 111.486 |
| RAN | 134.921 | 145.570 | 122.629 | 171.855 | 174.951 | 125.912 | 58.286 | 87.561 |
| NPM1 | 269.738 | 365.721 | 678.646 | 951.955 | 638.103 | 112.344 | 239.060 | 406.947 |
| FBL | 41.764 | 33.006 | 28.614 | 86.846 | 71.884 | 15.564 | 32.735 | 26.843 |
| NAP1L1 | 77.416 | 99.280 | 80.669 | 129.184 | 115.709 | 72.438 | 47.465 | 54.066 |
| SRSF1 | 63.478 | 53.877 | 60.656 | 57.290 | 74.446 | 54.660 | 54.479 | 63.431 |
| TCP1 | 45.996 | 57.677 | 106.328 | 127.597 | 117.059 | 84.311 | 47.467 | 59.159 |
| PWP1 | 19.601 | 17.064 | 22.417 | 24.844 | 17.720 | 22.311 | 12.149 | 14.267 |
| APEX1 | 38.752 | 41.385 | 77.729 | 62.752 | 79.463 | 38.972 | 29.060 | 52.581 |
| NME1 | 45.357 | 41.571 | 61.601 | 74.222 | 59.172 | 25.076 | 27.523 | 35.736 |
| EEF1B2 | 173.404 | 145.262 | 212.614 | 272.165 | 263.583 | 105.128 | 212.631 | 269.167 |
| RPS2 | 1022.110 | 576.564 | 1259.885 | 1584.527 | 942.869 | 224.013 | 849.440 | 418.960 |
| RPS3 | 157.636 | 85.077 | 400.948 | 284.136 | 312.008 | 89.329 | 250.386 | 134.312 |
| SRSF3 | 73.306 | 51.324 | 55.697 | 66.941 | 80.043 | 54.763 | 36.092 | 49.417 |
| GOT2 | 25.066 | 28.442 | 24.264 | 31.983 | 59.872 | 28.548 | 13.871 | 24.089 |
| COX5A | 33.548 | 41.172 | 76.563 | 105.154 | 75.938 | 43.906 | 32.410 | 30.054 |
| HNRNPA1 | 14.565 | 6.263 | 11.215 | 17.047 | 13.812 | 5.658 | 11.208 | 10.391 |
| EIF3J | 13.395 | 15.157 | 24.423 | 21.150 | 29.863 | 21.673 | 9.492 | 9.531 |
| HDAC2 | 17.592 | 6.526 | 26.679 | 23.361 | 32.109 | 10.879 | 12.957 | 12.957 |
| CNBP | 48.867 | 57.758 | 70.622 | 84.649 | 53.495 | 40.897 | 32.464 | 39.917 |
| NOLC1 | 29.051 | 29.041 | 45.513 | 47.125 | 50.661 | 35.449 | 28.407 | 26.036 |
| PRDX4 | 45.859 | 50.307 | 79.764 | 130.470 | 72.664 | 35.515 | 57.399 | 46.150 |
| EIF4E | 8.955 | 5.916 | 9.347 | 11.221 | 9.381 | 9.983 | 6.603 | 6.950 |
| RPLP0 | 1030.430 | 1101.970 | 1133.312 | 1559.634 | 844.967 | 535.573 | 840.059 | 709.218 |
| ABCE1 | 21.043 | 36.249 | 39.635 | 39.768 | 27.874 | 20.547 | 14.870 | 20.396 |
| LSM7 | 58.845 | 49.404 | 75.899 | 58.881 | 52.470 | 36.379 | 40.744 | 49.376 |
| CLNS1A | 54.480 | 39.324 | 87.667 | 76.846 | 78.641 | 67.280 | 54.215 | 39.160 |
| RSL1D1 | 15.909 | 23.842 | 29.892 | 31.013 | 23.716 | 16.210 | 18.946 | 20.151 |
| HNRNPC | 110.254 | 134.075 | 131.462 | 144.708 | 136.610 | 88.363 | 67.634 | 90.337 |
| H2AFZ | 356.196 | 140.999 | 176.603 | 302.064 | 210.055 | 188.604 | 91.297 | 159.725 |
| EPRS | 37.829 | 52.665 | 89.007 | 97.193 | 37.645 | 24.053 | 38.494 | 32.591 |
| SRPK1 | 19.899 | 25.215 | 30.508 | 32.608 | 35.306 | 18.142 | 16.585 | 19.425 |
| MAD2L1 | 53.242 | 45.961 | 45.730 | 50.097 | 65.573 | 57.392 | 20.466 | 36.290 |
| RPS6 | 681.900 | 440.453 | 1339.403 | 928.975 | 520.400 | 534.475 | 956.383 | 830.900 |
| RRP9 | 9.305 | 8.796 | 16.130 | 16.024 | 17.820 | 13.872 | 9.199 | 13.021 |
| GNL3 | 49.609 | 33.737 | 42.674 | 37.691 | 50.909 | 38.624 | 45.487 | 40.781 |
| SLC19A1 | 5.758 | 7.451 | 6.121 | 4.742 | 5.009 | 5.339 | 4.654 | 7.325 |
| TMEM97 | 16.796 | 10.144 | 27.174 | 26.457 | 58.973 | 24.463 | 15.576 | 75.893 |
| PES1 | 20.033 | 21.244 | 22.512 | 29.951 | 24.084 | 22.809 | 18.075 | 18.394 |
| EXOSC5 | 13.855 | 20.233 | 17.551 | 28.498 | 31.397 | 14.761 | 14.283 | 7.771 |
| NDUFAF4 | 5.139 | 7.105 | 8.148 | 9.231 | 8.826 | 4.621 | 3.059 | 3.543 |
| RABEPK | 6.428 | 10.803 | 17.277 | 10.899 | 12.057 | 9.239 | 5.852 | 7.887 |
| WDR74 | 18.033 | 26.597 | 31.794 | 13.403 | 34.750 | 21.016 | 18.898 | 19.967 |
| PLK4 | 14.632 | 8.814 | 8.362 | 8.770 | 13.374 | 10.956 | 4.303 | 9.639 |
| RCL1 | 7.593 | 6.866 | 10.737 | 9.348 | 6.272 | 8.191 | 9.711 | 9.909 |
| PPRC1 | 5.621 | 5.508 | 9.086 | 8.061 | 9.780 | 4.982 | 4.958 | 8.188 |
| MYC | 15.628 | 10.235 | 0.239 | 0.061 | 0.019 | 1.749 | 25.718 | 0.080 |

TABLE 6
Gene Expression in Neuroblastoma Cell Lines

| | Cell Line | | | | | | | |
|---|---|---|---|---|---|---|---|---|
| Gene | SMSSAN | CHP134 | CHP212 | COGN415 | COGN440 | COGN453 | COGN471 | COGN496 |
| MYCN | 1052.545 | 361.261 | 428.219 | 547.763 | 353.204 | 262.398 | 130.549 | 103.856 |
| MXI1 | 15.801 | 5.713 | 3.744 | 6.468 | 8.590 | 7.457 | 5.394 | 2.928 |
| MPST | 18.995 | 23.862 | 18.022 | 18.623 | 29.471 | 16.521 | 13.142 | 16.106 |
| MAX | 8.472 | 7.309 | 10.403 | 6.806 | 8.524 | 6.700 | 4.748 | 5.167 |
| SMARCA4 | 24.979 | 30.009 | 23.912 | 23.991 | 33.419 | 14.933 | 8.531 | 33.617 |
| SET | 183.833 | 120.677 | 177.164 | 124.031 | 182.296 | 113.963 | 54.715 | 180.816 |
| RAN | 237.991 | 100.416 | 146.405 | 131.536 | 124.957 | 119.591 | 52.483 | 156.808 |
| NPM1 | 330.598 | 328.709 | 573.200 | 508.468 | 450.145 | 934.236 | 143.702 | 334.676 |
| FBL | 65.160 | 39.306 | 59.378 | 55.573 | 17.099 | 44.674 | 11.882 | 39.649 |
| NAP1L1 | 99.898 | 64.607 | 79.063 | 62.513 | 48.502 | 71.087 | 34.345 | 65.018 |
| SRSF1 | 73.632 | 71.577 | 83.431 | 69.932 | 73.005 | 73.580 | 31.669 | 58.006 |
| TCP1 | 93.453 | 43.505 | 116.250 | 84.019 | 102.978 | 37.206 | 33.506 | 51.770 |
| PWP1 | 33.785 | 16.286 | 24.016 | 15.898 | 21.144 | 12.372 | 6.169 | 11.792 |
| APEX1 | 60.849 | 41.781 | 87.304 | 51.357 | 48.499 | 54.811 | 22.284 | 34.372 |
| NME1 | 89.419 | 73.456 | 138.645 | 64.895 | 47.910 | 47.734 | 23.719 | 51.020 |
| EEF1B2 | 237.280 | 177.484 | 316.856 | 227.287 | 264.466 | 351.645 | 91.332 | 147.219 |
| RPS2 | 901.235 | 669.311 | 1071.991 | 953.667 | 449.834 | 1142.570 | 164.547 | 554.467 |
| RPS3 | 159.796 | 141.019 | 219.089 | 183.733 | 136.633 | 245.011 | 45.049 | 90.963 |
| SRSF3 | 88.188 | 59.452 | 73.303 | 60.534 | 73.500 | 53.600 | 20.722 | 55.567 |
| GOT2 | 38.093 | 38.062 | 58.345 | 30.089 | 38.831 | 21.395 | 16.089 | 24.123 |
| COX5A | 73.599 | 46.291 | 53.384 | 63.128 | 42.288 | 75.592 | 15.064 | 44.642 |
| HNRNPA1 | 14.227 | 8.200 | 11.697 | 11.609 | 10.954 | 12.447 | 3.225 | 10.986 |
| EIF3J | 21.418 | 12.187 | 27.562 | 16.426 | 16.741 | 15.844 | 6.151 | 13.701 |
| HDAC2 | 31.825 | 16.497 | 17.432 | 22.045 | 14.650 | 19.515 | 6.683 | 16.122 |
| CNBP | 74.971 | 52.337 | 67.753 | 65.542 | 42.791 | 49.832 | 22.448 | 56.831 |
| NOLC1 | 46.566 | 34.499 | 49.614 | 30.409 | 32.811 | 17.403 | 15.254 | 20.432 |
| PRDX4 | 73.903 | 33.683 | 90.210 | 75.099 | 72.861 | 49.692 | 18.638 | 59.676 |
| EIF4E | 13.808 | 8.786 | 12.215 | 10.111 | 11.241 | 8.764 | 3.356 | 7.871 |
| RPLP0 | 1229.434 | 781.986 | 1025.132 | 779.648 | 540.764 | 1090.600 | 261.405 | 500.096 |
| ABCE1 | 38.386 | 31.622 | 49.974 | 27.810 | 27.127 | 21.807 | 10.873 | 19.202 |
| LSM7 | 71.095 | 54.340 | 71.131 | 67.678 | 83.515 | 56.046 | 10.557 | 43.420 |
| CLNS1A | 55.652 | 55.701 | 57.043 | 40.475 | 46.374 | 49.521 | 18.392 | 30.985 |
| RSL1D1 | 22.244 | 17.945 | 31.668 | 17.229 | 18.476 | 27.207 | 8.855 | 14.202 |
| HNRNPC | 155.693 | 119.605 | 138.719 | 100.563 | 115.097 | 109.197 | 39.659 | 113.366 |
| H2AFZ | 416.078 | 159.559 | 300.517 | 282.011 | 221.625 | 210.710 | 86.346 | 227.206 |
| EPRS | 62.475 | 71.602 | 50.962 | 34.761 | 54.272 | 41.996 | 15.318 | 29.725 |
| SRPK1 | 34.762 | 47.399 | 26.921 | 25.403 | 31.133 | 21.592 | 11.183 | 19.345 |
| MAD2L1 | 86.140 | 46.927 | 53.382 | 58.563 | 58.709 | 38.594 | 19.774 | 49.770 |
| RPS6 | 715.863 | 659.925 | 1062.872 | 453.430 | 758.988 | 1087.584 | 213.687 | 415.872 |
| RRP9 | 19.128 | 9.400 | 20.728 | 28.102 | 25.359 | 14.790 | 7.057 | 19.893 |
| GNL3 | 78.680 | 25.921 | 67.787 | 62.480 | 58.830 | 47.470 | 16.707 | 58.095 |
| SLC19A1 | 5.816 | 6.542 | 8.420 | 9.574 | 8.562 | 5.622 | 3.081 | 5.608 |
| TMEM97 | 42.636 | 32.947 | 21.800 | 46.014 | 57.352 | 24.752 | 11.998 | 39.522 |
| PES1 | 22.737 | 19.689 | 34.341 | 22.342 | 24.269 | 18.992 | 11.807 | 20.826 |
| EXOSC5 | 19.472 | 22.102 | 20.743 | 31.312 | 9.148 | 16.082 | 6.534 | 18.094 |
| NDUFAF4 | 9.274 | 7.075 | 8.890 | 7.616 | 8.643 | 5.494 | 2.816 | 6.751 |
| RABEPK | 13.711 | 29.649 | 18.115 | 10.711 | 12.203 | 7.488 | 4.436 | 8.744 |
| WDR74 | 23.982 | 24.041 | 26.924 | 23.551 | 29.809 | 19.720 | 10.560 | 16.631 |
| PLK4 | 15.168 | 6.686 | 12.025 | 9.934 | 9.213 | 8.640 | 4.146 | 9.571 |
| RCL1 | 13.756 | 8.132 | 10.222 | 7.272 | 9.914 | 11.189 | 4.026 | 11.674 |
| PPRC1 | 7.105 | 7.232 | 12.136 | 10.183 | 8.436 | 2.659 | 3.751 | 6.558 |
| MYC | 0.032 | 0.181 | 0.051 | 0.197 | 0.071 | 0.112 | 0.089 | 0.375 |

TABLE 7
Gene Expression in Neuroblastoma Cell Lines

| | Cell Line | | | | | | | |
|---|---|---|---|---|---|---|---|---|
| Gene | COGN519 | COGN534 | COGN549 | COGN557 | COGN561 | COGN573 | FELIX | HU.FETAL.BRAIN |
| MYCN | 794.052 | 8.810 | 5.183 | 101.220 | 301.107 | 479.777 | 3.104 | 8.937 |
| MXI1 | 8.130 | 3.406 | 1.912 | 4.055 | 7.686 | 5.135 | 6.769 | 7.589 |
| MPST | 27.141 | 7.556 | 3.863 | 8.060 | 21.038 | 10.277 | 6.282 | 4.076 |
| MAX | 9.748 | 7.626 | 4.036 | 3.444 | 7.331 | 4.978 | 7.678 | 5.642 |
| SMARCA4 | 32.056 | 6.173 | 9.694 | 15.906 | 27.326 | 12.911 | 13.229 | 14.703 |
| SET | 160.196 | 76.530 | 51.676 | 50.423 | 110.732 | 42.689 | 101.323 | 24.949 |
| RAN | 137.615 | 71.193 | 42.740 | 38.232 | 104.664 | 33.848 | 51.637 | 18.450 |
| NPM1 | 608.253 | 167.523 | 79.500 | 89.596 | 457.301 | 176.673 | 132.376 | 16.269 |
| FBL | 55.742 | 12.871 | 6.075 | 10.477 | 49.533 | 18.306 | 13.711 | 10.795 |
| NAP1L1 | 70.666 | 52.466 | 23.949 | 18.156 | 65.716 | 25.047 | 26.867 | 10.402 |
| SRSF1 | 80.199 | 39.536 | 29.719 | 27.952 | 69.178 | 17.731 | 56.601 | 11.392 |
| TCP1 | 134.756 | 30.188 | 30.645 | 25.145 | 38.405 | 23.575 | 54.465 | 22.679 |
| PWP1 | 17.440 | 14.889 | 6.677 | 6.025 | 14.408 | 4.987 | 12.552 | 6.254 |
| APEX1 | 52.860 | 29.789 | 12.301 | 19.745 | 70.716 | 13.554 | 17.773 | 13.620 |
| NME1 | 68.399 | 19.888 | 11.043 | 17.975 | 51.556 | 10.266 | 26.557 | 4.826 |
| EEF1B2 | 248.926 | 85.287 | 58.262 | 74.502 | 269.860 | 95.663 | 152.748 | 54.730 |
| RPS2 | 633.519 | 324.570 | 125.369 | 127.213 | 920.901 | 312.011 | 223.876 | 313.180 |
| RPS3 | 200.581 | 73.532 | 41.663 | 38.281 | 223.115 | 76.257 | 66.797 | 37.558 |
| SRSF3 | 75.810 | 43.234 | 23.492 | 22.046 | 57.933 | 16.463 | 53.636 | 11.626 |
| GOT2 | 34.883 | 21.236 | 13.728 | 12.898 | 19.395 | 12.957 | 31.337 | 10.827 |
| COX5A | 40.390 | 27.993 | 11.621 | 13.712 | 37.057 | 17.420 | 20.130 | 18.247 |
| HNRNPA1 | 12.044 | 8.569 | 3.202 | 3.728 | 14.639 | 4.152 | 4.970 | 2.453 |
| EIF3J | 16.264 | 8.932 | 5.789 | 4.492 | 12.500 | 6.506 | 7.528 | 4.122 |
| HDAC2 | 18.901 | 6.849 | 6.678 | 7.282 | 15.048 | 5.820 | 15.225 | 4.313 |
| CNBP | 69.570 | 36.758 | 18.959 | 17.264 | 44.775 | 22.385 | 32.251 | 11.210 |
| NOLC1 | 44.393 | 24.010 | 6.727 | 9.724 | 30.491 | 18.924 | 22.242 | 6.085 |
| PRDX4 | 86.200 | 22.201 | 12.696 | 14.055 | 55.307 | 16.996 | 16.915 | 5.837 |
| EIF4E | 10.443 | 5.316 | 4.719 | 4.269 | 7.500 | 3.640 | 5.783 | 3.606 |
| RPLP0 | 632.465 | 405.092 | 192.402 | 201.090 | 904.914 | 371.373 | 231.213 | 180.096 |
| ABCE1 | 36.381 | 9.168 | 8.838 | 8.358 | 22.769 | 10.042 | 11.982 | 2.968 |
| LSM7 | 66.028 | 29.601 | 12.082 | 22.912 | 39.968 | 19.402 | 28.653 | 14.123 |
| CLNS1A | 56.695 | 27.223 | 25.322 | 15.404 | 41.430 | 16.254 | 39.127 | 14.169 |
| RSL1D1 | 24.172 | 11.183 | 8.112 | 6.519 | 24.212 | 10.782 | 8.272 | 3.692 |
| HNRNPC | 137.274 | 76.693 | 49.274 | 36.414 | 105.270 | 34.934 | 66.890 | 36.212 |
| H2AFZ | 281.630 | 124.242 | 79.609 | 81.234 | 174.340 | 46.461 | 85.329 | 25.376 |
| EPRS | 32.557 | 28.051 | 20.233 | 10.813 | 28.886 | 13.060 | 19.648 | 4.518 |
| SRPK1 | 52.945 | 16.312 | 9.504 | 10.911 | 16.672 | 11.392 | 20.550 | 8.787 |
| MAD2L1 | 59.489 | 16.850 | 18.750 | 15.589 | 31.595 | 11.307 | 22.110 | 0.780 |
| RPS6 | 1530.287 | 275.273 | 185.470 | 182.728 | 890.036 | 278.583 | 396.643 | 140.580 |
| RRP9 | 19.670 | 8.392 | 2.399 | 3.103 | 15.660 | 7.911 | 7.366 | 2.953 |
| GNL3 | 39.896 | 20.911 | 15.351 | 12.421 | 50.328 | 21.111 | 18.289 | 4.892 |
| SLC19A1 | 10.116 | 3.105 | 1.653 | 3.322 | 8.728 | 2.797 | 2.458 | 2.608 |
| TMEM97 | 51.039 | 15.853 | 13.700 | 15.283 | 58.357 | 9.419 | 14.608 | 2.280 |
| PES1 | 33.842 | 10.856 | 5.609 | 5.581 | 18.437 | 6.666 | 14.980 | 6.336 |
| EXOSC5 | 30.362 | 7.250 | 2.750 | 4.626 | 13.185 | 8.096 | 13.998 | 4.238 |
| NDUFAF4 | 8.893 | 4.193 | 2.457 | 3.594 | 5.460 | 2.714 | 4.204 | 2.628 |
| RABEPK | 14.571 | 4.548 | 1.926 | 3.176 | 9.208 | 3.290 | 6.417 | 4.183 |
| WDR74 | 35.286 | 20.001 | 5.818 | 6.771 | 20.560 | 11.610 | 12.794 | 5.419 |
| PLK4 | 10.671 | 4.599 | 4.541 | 4.423 | 6.838 | 1.898 | 4.715 | 0.272 |
| RCL1 | 20.763 | 4.069 | 3.446 | 2.726 | 11.234 | 4.609 | 6.041 | 2.779 |
| PPRC1 | 14.800 | 3.551 | 1.171 | 2.997 | 7.863 | 5.954 | 2.972 | 2.364 |
| MYC | 0.031 | 0.012 | 0.726 | 0.190 | 0.059 | 0.096 | 0.809 | 3.286 |

TABLE 8
Gene Expression in Neuroblastoma Cell Lines

| | Cell Line | | | | | | | |
|---|---|---|---|---|---|---|---|---|
| Gene | IMR32 | IMR5 | KELLY | LAN5 | LAN6 | NB1 | NB16 | NB1643 |
| MYCN | 152.787 | 289.376 | 644.877 | 786.076 | 2.771 | 273.312 | 1.773 | 395.478 |
| MXI1 | 2.713 | 9.829 | 12.417 | 14.203 | 3.931 | 4.723 | 5.313 | 7.050 |
| MPST | 6.289 | 22.435 | 25.194 | 18.790 | 12.192 | 15.291 | 20.075 | 16.288 |
| MAX | 3.517 | 7.116 | 7.375 | 10.090 | 6.883 | 7.520 | 4.398 | 5.782 |
| SMARCA4 | 15.078 | 24.281 | 28.621 | 29.178 | 19.547 | 25.872 | 19.758 | 23.128 |
| SET | 63.172 | 138.895 | 130.513 | 134.025 | 63.794 | 86.567 | 135.728 | 78.655 |
| RAN | 78.151 | 202.757 | 171.237 | 120.032 | 113.312 | 103.035 | 131.296 | 81.214 |
| NPM1 | 238.330 | 798.954 | 858.626 | 205.326 | 163.451 | 269.024 | 674.899 | 344.213 |
| FBL | 16.002 | 63.071 | 81.565 | 29.170 | 18.688 | 29.823 | 53.289 | 35.961 |
| NAP1L1 | 44.521 | 53.536 | 116.150 | 48.411 | 27.104 | 19.377 | 87.861 | 59.951 |
| SRSF1 | 45.149 | 73.127 | 70.990 | 73.551 | 49.209 | 49.215 | 52.011 | 67.205 |
| TCP1 | 52.086 | 161.813 | 116.858 | 71.357 | 23.796 | 55.310 | 137.966 | 50.952 |
| PWP1 | 10.893 | 19.223 | 18.936 | 15.642 | 13.408 | 19.701 | 20.508 | 13.009 |
| APEX1 | 17.288 | 57.070 | 73.264 | 51.480 | 29.863 | 57.939 | 50.207 | 46.086 |
| NME1 | 34.053 | 60.731 | 98.757 | 66.459 | 18.315 | 38.346 | 36.024 | 53.924 |
| EEF1B2 | 118.776 | 293.454 | 202.378 | 204.598 | 107.716 | 169.655 | 253.030 | 230.727 |
| RPS2 | 280.921 | 1388.890 | 1473.079 | 442.038 | 450.411 | 632.388 | 1073.400 | 778.175 |
| RPS3 | 52.836 | 211.758 | 296.795 | 104.097 | 57.131 | 108.832 | 226.228 | 162.933 |
| SRSF3 | 36.120 | 64.501 | 80.308 | 66.980 | 39.207 | 50.527 | 72.193 | 46.463 |
| GOT2 | 10.508 | 27.760 | 29.993 | 41.594 | 17.369 | 34.984 | 20.908 | 19.293 |
| COX5A | 22.799 | 62.134 | 77.767 | 35.845 | 39.190 | 59.953 | 70.198 | 42.743 |
| HNRNPA1 | 5.221 | 12.337 | 11.378 | 7.442 | 7.328 | 8.545 | 9.196 | 11.145 |
| EIF3J | 7.883 | 24.902 | 23.573 | 14.586 | 10.339 | 15.267 | 12.007 | 14.404 |
| HDAC2 | 12.523 | 20.967 | 26.737 | 16.895 | 8.855 | 11.834 | 21.784 | 17.909 |
| CNBP | 27.148 | 61.016 | 97.445 | 52.659 | 40.264 | 39.175 | 68.325 | 32.738 |
| NOLC1 | 17.572 | 69.055 | 39.070 | 37.901 | 23.849 | 37.701 | 45.368 | 34.006 |
| PRDX4 | 17.252 | 65.462 | 76.354 | 62.676 | 31.549 | 41.425 | 50.028 | 40.764 |
| EIF4E | 3.985 | 10.904 | 13.547 | 11.169 | 5.322 | 9.273 | 8.706 | 6.182 |
| RPLP0 | 503.448 | 1198.520 | 1281.980 | 602.731 | 588.309 | 469.627 | 962.850 | 731.842 |
| ABCE1 | 13.558 | 43.890 | 41.760 | 30.447 | 11.900 | 22.272 | 35.889 | 21.347 |
| LSM7 | 21.395 | 57.444 | 60.639 | 40.852 | 29.235 | 57.274 | 46.034 | 31.504 |
| CLNS1A | 20.097 | 66.135 | 59.210 | 49.477 | 18.070 | 37.196 | 40.038 | 32.964 |
| RSL1D1 | 9.994 | 27.430 | 31.263 | 19.104 | 11.017 | 15.463 | 28.190 | 18.171 |
| HNRNPC | 55.039 | 159.326 | 164.688 | 109.139 | 59.790 | 78.901 | 136.915 | 78.280 |
| H2AFZ | 106.930 | 253.609 | 320.392 | 227.570 | 111.491 | 152.085 | 194.739 | 156.199 |
| EPRS | 21.757 | 80.830 | 98.908 | 26.632 | 33.793 | 47.492 | 43.920 | 44.941 |
| SRPK1 | 14.852 | 31.948 | 41.457 | 27.984 | 10.542 | 15.978 | 13.831 | 16.636 |
| MAD2L1 | 26.521 | 52.777 | 63.316 | 77.571 | 16.875 | 34.188 | 36.258 | 35.576 |
| RPS6 | 297.448 | 998.870 | 1146.248 | 562.849 | 208.909 | 553.788 | 658.861 | 571.272 |
| RRP9 | 7.285 | 22.666 | 30.884 | 13.149 | 4.592 | 10.683 | 20.592 | 9.485 |
| GNL3 | 24.927 | 79.642 | 88.163 | 60.336 | 9.748 | 42.126 | 60.193 | 39.119 |
| SLC19A1 | 3.178 | 6.122 | 5.369 | 9.145 | 5.015 | 5.946 | 5.580 | 6.145 |
| TMEM97 | 19.816 | 34.449 | 34.775 | 42.302 | 7.832 | 28.257 | 21.346 | 33.486 |
| PES1 | 9.081 | 21.142 | 42.574 | 22.832 | 14.168 | 17.484 | 29.885 | 19.837 |
| EXOSC5 | 5.770 | 21.173 | 35.409 | 16.528 | 11.884 | 13.631 | 16.300 | 10.634 |
| NDUFAF4 | 4.633 | 7.217 | 11.647 | 7.317 | 1.713 | 4.747 | 7.054 | 4.423 |
| RABEPK | 8.915 | 12.796 | 12.046 | 8.328 | 5.761 | 9.319 | 10.878 | 5.531 |
| WDR74 | 10.190 | 20.315 | 29.982 | 25.173 | 16.885 | 18.694 | 20.459 | 14.843 |
| PLK4 | 5.397 | 10.545 | 9.354 | 12.452 | 5.744 | 7.734 | 9.020 | 8.053 |
| RCL1 | 5.598 | 13.220 | 15.221 | 11.885 | 8.352 | 7.967 | 6.135 | 8.808 |
| PPRC1 | 5.446 | 11.739 | 10.389 | 10.359 | 5.747 | 8.366 | 7.368 | 9.473 |
| MYC | 0.061 | 0.000 | 0.025 | 0.070 | 2.030 | 0.018 | 17.040 | 0.022 |

TABLE 9
Gene Expression in Neuroblastoma Cell Lines

| | Cell Line | | | | | | | |
|---|---|---|---|---|---|---|---|---|
| Gene | NB1691 | NB69 | NBEBC1 | NBLS | NBSD | NGP | NLF | NMB |
| MYCN | 108.942 | 0.570 | 8.053 | 31.970 | 280.671 | 700.038 | 41.178 | 208.079 |
| MXI1 | 9.804 | 6.521 | 1.992 | 8.436 | 7.851 | 16.084 | 4.626 | 6.431 |
| MPST | 7.766 | 13.316 | 11.483 | 14.615 | 15.180 | 37.451 | 25.894 | 14.616 |
| MAX | 4.833 | 4.516 | 8.940 | 7.524 | 5.440 | 11.075 | 6.008 | 6.563 |
| SMARCA4 | 19.434 | 25.503 | 16.873 | 29.981 | 25.269 | 38.761 | 24.336 | 19.479 |
| SET | 66.152 | 126.079 | 136.868 | 108.545 | 115.980 | 159.194 | 174.647 | 98.419 |
| RAN | 75.750 | 134.374 | 128.530 | 129.690 | 83.661 | 135.119 | 124.207 | 84.081 |
| NPM1 | 283.493 | 520.328 | 172.879 | 290.033 | 174.317 | 633.101 | 536.492 | 210.718 |
| FBL | 44.882 | 59.380 | 14.099 | 42.532 | 19.114 | 128.548 | 35.819 | 18.353 |
| NAP1L1 | 51.397 | 69.348 | 47.646 | 49.118 | 44.236 | 119.605 | 66.924 | 57.160 |
| SRSF1 | 45.043 | 71.808 | 61.972 | 58.673 | 44.853 | 69.515 | 83.868 | 44.756 |
| TCP1 | 67.903 | 97.093 | 70.977 | 81.985 | 95.451 | 95.373 | 68.020 | 41.944 |
| PWP1 | 17.512 | 27.512 | 16.664 | 12.400 | 16.041 | 26.345 | 9.771 | 9.760 |
| APEX1 | 38.386 | 33.489 | 31.425 | 51.350 | 38.401 | 64.015 | 57.634 | 34.085 |
| NME1 | 15.865 | 91.254 | 29.329 | 39.512 | 22.743 | 61.198 | 51.470 | 28.882 |
| EEF1B2 | 129.882 | 208.373 | 131.733 | 271.322 | 116.874 | 302.958 | 185.460 | 112.177 |
| RPS2 | 633.882 | 1019.556 | 226.423 | 683.637 | 281.817 | 1560.447 | 590.705 | 426.039 |
| RPS3 | 131.120 | 187.741 | 71.989 | 154.110 | 83.618 | 450.528 | 124.086 | 85.620 |
| SRSF3 | 44.675 | 55.682 | 63.375 | 73.050 | 48.882 | 68.234 | 76.693 | 37.673 |
| GOT2 | 29.545 | 38.090 | 24.616 | 31.543 | 26.724 | 47.084 | 20.408 | 22.881 |
| COX5A | 27.792 | 75.156 | 21.845 | 87.663 | 27.973 | 80.267 | 39.209 | 13.302 |
| HNRNPA1 | 9.213 | 11.605 | 6.557 | 11.269 | 7.105 | 13.131 | 6.869 | 6.916 |
| EIF3J | 9.366 | 24.852 | 8.526 | 23.495 | 9.955 | 29.377 | 15.650 | 8.792 |
| HDAC2 | 19.943 | 16.985 | 11.832 | 18.035 | 17.503 | 33.074 | 16.778 | 10.380 |
| CNBP | 29.646 | 63.436 | 52.116 | 47.912 | 33.003 | 81.891 | 55.042 | 35.940 |
| NOLC1 | 18.068 | 53.676 | 16.640 | 40.268 | 20.845 | 37.439 | 27.843 | 34.312 |
| PRDX4 | 61.676 | 66.110 | 36.402 | 34.821 | 39.220 | 97.180 | 60.956 | 31.469 |
| EIF4E | 4.246 | 4.811 | 5.034 | 10.413 | 5.540 | 10.285 | 9.198 | 5.677 |
| RPLP0 | 1301.165 | 836.921 | 395.440 | 816.989 | 420.175 | 1343.204 | 578.184 | 556.801 |
| ABCE1 | 13.528 | 39.084 | 13.530 | 21.452 | 15.096 | 39.391 | 27.804 | 16.931 |
| LSM7 | 29.487 | 56.694 | 31.284 | 72.155 | 37.361 | 85.033 | 64.894 | 27.083 |
| CLNS1A | 25.349 | 47.023 | 46.765 | 57.557 | 41.381 | 81.391 | 47.933 | 38.426 |
| RSL1D1 | 14.112 | 24.445 | 13.626 | 23.160 | 16.583 | 30.095 | 24.793 | 20.007 |
| HNRNPC | 79.461 | 82.854 | 122.780 | 117.717 | 71.388 | 136.953 | 152.445 | 75.643 |
| H2AFZ | 113.922 | 134.246 | 116.199 | 172.637 | 103.219 | 151.166 | 231.424 | 133.199 |
| EPRS | 42.195 | 65.644 | 34.239 | 29.209 | 21.656 | 97.886 | 25.137 | 18.994 |
| SRPK1 | 16.118 | 22.079 | 18.090 | 18.618 | 32.468 | 36.407 | 19.729 | 20.213 |
| MAD2L1 | 21.602 | 25.412 | 23.037 | 38.104 | 28.878 | 37.439 | 48.682 | 32.170 |
| RPS6 | 727.538 | 833.568 | 458.491 | 705.523 | 383.252 | 1494.047 | 977.577 | 498.226 |
| RRP9 | 7.518 | 21.456 | 15.819 | 12.911 | 6.769 | 16.686 | 15.628 | 7.699 |
| GNL3 | 25.077 | 58.937 | 40.853 | 41.276 | 19.567 | 30.549 | 50.108 | 20.833 |
| SLC19A1 | 2.499 | 7.542 | 3.053 | 4.917 | 6.301 | 9.593 | 5.201 | 5.139 |
| TMEM97 | 24.589 | 38.714 | 25.596 | 21.549 | 31.739 | 38.271 | 26.739 | 26.456 |
| PES1 | 9.790 | 31.823 | 28.206 | 23.052 | 10.571 | 42.033 | 30.740 | 11.787 |
| EXOSC5 | 11.092 | 26.016 | 11.567 | 22.966 | 8.499 | 34.984 | 17.533 | 12.371 |
| NDUFAF4 | 6.528 | 7.253 | 3.841 | 5.130 | 4.370 | 11.826 | 5.600 | 3.298 |
| RABEPK | 5.302 | 5.098 | 7.386 | 7.494 | 6.587 | 16.854 | 16.607 | 4.898 |
| WDR74 | 9.561 | 28.162 | 17.464 | 21.796 | 17.716 | 27.897 | 27.978 | 15.692 |
| PLK4 | 4.821 | 4.445 | 6.631 | 10.431 | 5.713 | 8.999 | 11.918 | 6.018 |
| RCL1 | 5.786 | 12.737 | 13.859 | 11.250 | 6.609 | 9.513 | 7.593 | 5.811 |
| PPRC1 | 4.690 | 11.450 | 3.420 | 7.492 | 4.474 | 9.859 | 7.438 | 5.765 |
| MYC | 0.122 | 165.137 | 0.115 | 0.191 | 0.011 | 0.118 | 0.134 | 0.010 |

TABLE 10
Gene Expression in Neuroblastoma Cell Lines

| | Cell Line | | | | | | | |
|---|---|---|---|---|---|---|---|---|
| Gene | RPE1 | SHSY5Y | SKNAS | SKNBE2 | SKNBE2C | SKNDZ | SKNFI | SKNSH |
| MYCN | 0.085 | 1.451 | 0.046 | 695.209 | 520.553 | 256.233 | 18.155 | 4.962 |
| MXI1 | 4.625 | 5.327 | 5.760 | 16.499 | 7.278 | 9.237 | 7.845 | 12.631 |
| MPST | 10.892 | 20.426 | 14.359 | 41.759 | 27.022 | 34.107 | 10.317 | 18.931 |
| MAX | 7.467 | 7.821 | 9.033 | 8.825 | 6.165 | 5.062 | 9.029 | 7.981 |
| SMARCA4 | 9.593 | 26.997 | 16.545 | 33.665 | 25.484 | 29.151 | 7.832 | 16.265 |
| SET | 96.177 | 127.018 | 89.213 | 133.778 | 120.018 | 161.399 | 108.930 | 77.062 |
| RAN | 122.168 | 134.921 | 145.570 | 122.629 | 171.855 | 174.951 | 125.912 | 58.286 |
| NPM1 | 342.794 | 269.738 | 365.721 | 678.646 | 951.955 | 638.103 | 112.344 | 239.060 |
| FBL | 28.950 | 41.764 | 33.006 | 28.614 | 86.846 | 71.884 | 15.564 | 32.735 |
| NAP1L1 | 64.678 | 77.416 | 99.280 | 80.669 | 129.184 | 115.709 | 72.438 | 47.465 |
| SRSF1 | 44.705 | 63.478 | 53.877 | 60.656 | 57.290 | 74.446 | 54.660 | 54.479 |
| TCP1 | 74.795 | 45.996 | 57.677 | 106.328 | 127.597 | 117.059 | 84.311 | 47.467 |
| PWP1 | 16.193 | 19.601 | 17.064 | 22.417 | 24.844 | 17.720 | 22.311 | 12.149 |
| APEX1 | 37.280 | 38.752 | 41.385 | 77.729 | 62.752 | 79.463 | 38.972 | 29.060 |
| NME1 | 24.668 | 45.357 | 41.571 | 61.601 | 74.222 | 59.172 | 25.076 | 27.523 |
| EEF1B2 | 147.368 | 173.404 | 145.262 | 212.614 | 272.165 | 263.583 | 105.128 | 212.631 |
| RPS2 | 770.216 | 1022.110 | 576.564 | 1259.885 | 1584.527 | 942.869 | 224.013 | 849.440 |
| RPS3 | 121.718 | 157.636 | 85.077 | 400.948 | 284.136 | 312.008 | 89.329 | 250.386 |
| SRSF3 | 53.003 | 73.306 | 51.324 | 55.697 | 66.941 | 80.043 | 54.763 | 36.092 |
| GOT2 | 29.405 | 25.066 | 28.442 | 24.264 | 31.983 | 59.872 | 28.548 | 13.871 |
| COX5A | 34.231 | 33.548 | 41.172 | 76.563 | 105.154 | 75.938 | 43.906 | 32.410 |
| HNRNPA1 | 7.216 | 14.565 | 6.263 | 11.215 | 17.047 | 13.812 | 5.658 | 11.208 |
| EIF3J | 13.337 | 13.395 | 15.157 | 24.423 | 21.150 | 29.863 | 21.673 | 9.492 |
| HDAC2 | 12.498 | 17.592 | 6.526 | 26.679 | 23.361 | 32.109 | 10.879 | 12.957 |
| CNBP | 28.435 | 48.867 | 57.758 | 70.622 | 84.649 | 53.495 | 40.897 | 32.464 |
| NOLC1 | 26.127 | 29.051 | 29.041 | 45.513 | 47.125 | 50.661 | 35.449 | 28.407 |
| PRDX4 | 46.293 | 45.859 | 50.307 | 79.764 | 130.470 | 72.664 | 35.515 | 57.399 |
| EIF4E | 8.980 | 8.955 | 5.916 | 9.347 | 11.221 | 9.381 | 9.983 | 6.603 |
| RPLP0 | 774.252 | 1030.430 | 1101.970 | 1133.312 | 1559.634 | 844.967 | 535.573 | 840.059 |
| ABCE1 | 27.624 | 21.043 | 36.249 | 39.635 | 39.768 | 27.874 | 20.547 | 14.870 |
| LSM7 | 24.766 | 58.845 | 49.404 | 75.899 | 58.881 | 52.470 | 36.379 | 40.744 |
| CLNS1A | 26.019 | 54.480 | 39.324 | 87.667 | 76.846 | 78.641 | 67.280 | 54.215 |
| RSL1D1 | 11.502 | 15.909 | 23.842 | 29.892 | 31.013 | 23.716 | 16.210 | 18.946 |
| HNRNPC | 91.783 | 110.254 | 134.075 | 131.462 | 144.708 | 136.610 | 88.363 | 67.634 |
| H2AFZ | 300.753 | 356.196 | 140.999 | 176.603 | 302.064 | 210.055 | 188.604 | 91.297 |
| EPRS | 26.697 | 37.829 | 52.665 | 89.007 | 97.193 | 37.645 | 24.053 | 38.494 |
| SRPK1 | 21.594 | 19.899 | 25.215 | 30.508 | 32.608 | 35.306 | 18.142 | 16.585 |
| MAD2L1 | 42.088 | 53.242 | 45.961 | 45.730 | 50.097 | 65.573 | 57.392 | 20.466 |
| RPS6 | 661.175 | 681.900 | 440.453 | 1339.403 | 928.975 | 520.400 | 534.475 | 956.383 |
| RRP9 | 7.670 | 9.305 | 8.796 | 16.130 | 16.024 | 17.820 | 13.872 | 9.199 |
| GNL3 | 25.810 | 49.609 | 33.737 | 42.674 | 37.691 | 50.909 | 38.624 | 45.487 |
| SLC19A1 | 2.936 | 5.758 | 7.451 | 6.121 | 4.742 | 5.009 | 5.339 | 4.654 |
| TMEM97 | 7.626 | 16.796 | 10.144 | 27.174 | 26.457 | 58.973 | 24.463 | 15.576 |
| PES1 | 18.276 | 20.033 | 21.244 | 22.512 | 29.951 | 24.084 | 22.809 | 18.075 |
| EXOSC5 | 5.873 | 13.855 | 20.233 | 17.551 | 28.498 | 31.397 | 14.761 | 14.283 |
| NDUFAF4 | 2.520 | 5.139 | 7.105 | 8.148 | 9.231 | 8.826 | 4.621 | 3.059 |
| RABEPK | 7.506 | 6.428 | 10.803 | 17.277 | 10.899 | 12.057 | 9.239 | 5.852 |
| WDR74 | 8.365 | 18.033 | 26.597 | 31.794 | 13.403 | 34.750 | 21.016 | 18.898 |
| PLK4 | 9.744 | 14.632 | 8.814 | 8.362 | 8.770 | 13.374 | 10.956 | 4.303 |
| RCL1 | 2.926 | 7.593 | 6.866 | 10.737 | 9.348 | 6.272 | 8.191 | 9.711 |
| PPRC1 | 5.290 | 5.621 | 5.508 | 9.086 | 8.061 | 9.780 | 4.982 | 4.958 |
| MYC | 18.582 | 15.628 | 10.235 | 0.239 | 0.061 | 0.019 | 1.749 | 25.718 |

TABLE 11
Gene Expression in Neuroblastoma Cell Lines

| | Cell Line | | | | | | | |
|---|---|---|---|---|---|---|---|---|
| Gene | SMSKAN | SMSSAN | CHP134 | CHP212 | COGN415 | COGN440 | COGN453 | COGN471 |
| MYCN | 301.818 | 1052.545 | 361.261 | 428.219 | 547.763 | 353.204 | 262.398 | 130.549 |
| MXI1 | 11.047 | 15.801 | 5.713 | 3.744 | 6.468 | 8.590 | 7.457 | 5.394 |
| MPST | 14.775 | 18.995 | 23.862 | 18.022 | 18.623 | 29.471 | 16.521 | 13.142 |
| MAX | 6.933 | 8.472 | 7.309 | 10.403 | 6.806 | 8.524 | 6.700 | 4.748 |
| SMARCA4 | 25.926 | 24.979 | 30.009 | 23.912 | 23.991 | 33.419 | 14.933 | 8.531 |
| SET | 111.486 | 183.833 | 120.677 | 177.164 | 124.031 | 182.296 | 113.963 | 54.715 |
| RAN | 87.561 | 237.991 | 100.416 | 146.405 | 131.536 | 124.957 | 119.591 | 52.483 |
| NPM1 | 406.947 | 330.598 | 328.709 | 573.200 | 508.468 | 450.145 | 934.236 | 143.702 |
| FBL | 26.843 | 65.160 | 39.306 | 59.378 | 55.573 | 17.099 | 44.674 | 11.882 |
| NAP1L1 | 54.066 | 99.898 | 64.607 | 79.063 | 62.513 | 48.502 | 71.087 | 34.345 |
| SRSF1 | 63.431 | 73.632 | 71.577 | 83.431 | 69.932 | 73.005 | 73.580 | 31.669 |
| TCP1 | 59.159 | 93.453 | 43.505 | 116.250 | 84.019 | 102.978 | 37.206 | 33.506 |
| PWP1 | 14.267 | 33.785 | 16.286 | 24.016 | 15.898 | 21.144 | 12.372 | 6.169 |
| APEX1 | 52.581 | 60.849 | 41.781 | 87.304 | 51.357 | 48.499 | 54.811 | 22.284 |
| NME1 | 35.736 | 89.419 | 73.456 | 138.645 | 64.895 | 47.910 | 47.734 | 23.719 |
| EEF1B2 | 269.167 | 237.280 | 177.484 | 316.856 | 227.287 | 264.466 | 351.645 | 91.332 |
| RPS2 | 418.960 | 901.235 | 669.311 | 1071.991 | 953.667 | 449.834 | 1142.570 | 164.547 |
| RPS3 | 134.312 | 159.796 | 141.019 | 219.089 | 183.733 | 136.633 | 245.011 | 45.049 |
| SRSF3 | 49.417 | 88.188 | 59.452 | 73.303 | 60.534 | 73.500 | 53.600 | 20.722 |
| GOT2 | 24.089 | 38.093 | 38.062 | 58.345 | 30.089 | 38.831 | 21.395 | 16.089 |
| COX5A | 30.054 | 73.599 | 46.291 | 53.384 | 63.128 | 42.288 | 75.592 | 15.064 |
| HNRNPA1 | 10.391 | 14.227 | 8.200 | 11.697 | 11.609 | 10.954 | 12.447 | 3.225 |
| EIF3J | 9.531 | 21.418 | 12.187 | 27.562 | 16.426 | 16.741 | 15.844 | 6.151 |
| HDAC2 | 12.957 | 31.825 | 16.497 | 17.432 | 22.045 | 14.650 | 19.515 | 6.683 |
| CNBP | 39.917 | 74.971 | 52.337 | 67.753 | 65.542 | 42.791 | 49.832 | 22.448 |
| NOLC1 | 26.036 | 46.566 | 34.499 | 49.614 | 30.409 | 32.811 | 17.403 | 15.254 |
| PRDX4 | 46.150 | 73.903 | 33.683 | 90.210 | 75.099 | 72.861 | 49.692 | 18.638 |
| EIF4E | 6.950 | 13.808 | 8.786 | 12.215 | 10.111 | 11.241 | 8.764 | 3.356 |
| RPLP0 | 709.218 | 1229.434 | 781.986 | 1025.132 | 779.648 | 540.764 | 1090.600 | 261.405 |
| ABCE1 | 20.396 | 38.386 | 31.622 | 49.974 | 27.810 | 27.127 | 21.807 | 10.873 |
| LSM7 | 49.376 | 71.095 | 54.340 | 71.131 | 67.678 | 83.515 | 56.046 | 10.557 |
| CLNS1A | 39.160 | 55.652 | 55.701 | 57.043 | 40.475 | 46.374 | 49.521 | 18.392 |
| RSL1D1 | 20.151 | 22.244 | 17.945 | 31.668 | 17.229 | 18.476 | 27.207 | 8.855 |
| HNRNPC | 90.337 | 155.693 | 119.605 | 138.719 | 100.563 | 115.097 | 109.197 | 39.659 |
| H2AFZ | 159.725 | 416.078 | 159.559 | 300.517 | 282.011 | 221.625 | 210.710 | 86.346 |
| EPRS | 32.591 | 62.475 | 71.602 | 50.962 | 34.761 | 54.272 | 41.996 | 15.318 |
| SRPK1 | 19.425 | 34.762 | 47.399 | 26.921 | 25.403 | 31.133 | 21.592 | 11.183 |
| MAD2L1 | 36.290 | 86.140 | 46.927 | 53.382 | 58.563 | 58.709 | 38.594 | 19.774 |
| RPS6 | 830.900 | 715.863 | 659.925 | 1062.872 | 453.430 | 758.988 | 1087.584 | 213.687 |
| RRP9 | 13.021 | 19.128 | 9.400 | 20.728 | 28.102 | 25.359 | 14.790 | 7.057 |
| GNL3 | 40.781 | 78.680 | 25.921 | 67.787 | 62.480 | 58.830 | 47.470 | 16.707 |
| SLC19A1 | 7.325 | 5.816 | 6.542 | 8.420 | 9.574 | 8.562 | 5.622 | 3.081 |
| TMEM97 | 75.893 | 42.636 | 32.947 | 21.800 | 46.014 | 57.352 | 24.752 | 11.998 |
| PES1 | 18.394 | 22.737 | 19.689 | 34.341 | 22.342 | 24.269 | 18.992 | 11.807 |
| EXOSC5 | 7.771 | 19.472 | 22.102 | 20.743 | 31.312 | 9.148 | 16.082 | 6.534 |
| NDUFAF4 | 3.543 | 9.274 | 7.075 | 8.890 | 7.616 | 8.643 | 5.494 | 2.816 |
| RABEPK | 7.887 | 13.711 | 29.649 | 18.115 | 10.711 | 12.203 | 7.488 | 4.436 |
| WDR74 | 19.967 | 23.982 | 24.041 | 26.924 | 23.551 | 29.809 | 19.720 | 10.560 |
| PLK4 | 9.639 | 15.168 | 6.686 | 12.025 | 9.934 | 9.213 | 8.640 | 4.146 |
| RCL1 | 9.909 | 13.756 | 8.132 | 10.222 | 7.272 | 9.914 | 11.189 | 4.026 |
| PPRC1 | 8.188 | 7.105 | 7.232 | 12.136 | 10.183 | 8.436 | 2.659 | 3.751 |
| MYC | 0.080 | 0.032 | 0.181 | 0.051 | 0.197 | 0.071 | 0.112 | 0.089 |

TABLE 12
Gene Expression in Neuroblastoma Cell Lines

| | Cell Line | | | | | | | |
|---|---|---|---|---|---|---|---|---|
| Gene | COGN496 | COGN519 | COGN534 | COGN549 | COGN557 | COGN561 | COGN573 | FELIX |
| MYCN | 103.856 | 794.052 | 8.810 | 5.183 | 101.220 | 301.107 | 479.777 | 3.104 |
| MXI1 | 2.928 | 8.130 | 3.406 | 1.912 | 4.055 | 7.686 | 5.135 | 6.769 |
| MPST | 16.106 | 27.141 | 7.556 | 3.863 | 8.060 | 21.038 | 10.277 | 6.282 |
| MAX | 5.167 | 9.748 | 7.626 | 4.036 | 3.444 | 7.331 | 4.978 | 7.678 |
| SMARCA4 | 33.617 | 32.056 | 6.173 | 9.694 | 15.906 | 27.326 | 12.911 | 13.229 |
| SET | 180.816 | 160.196 | 76.530 | 51.676 | 50.423 | 110.732 | 42.689 | 101.323 |
| RAN | 156.808 | 137.615 | 71.193 | 42.740 | 38.232 | 104.664 | 33.848 | 51.637 |
| NPM1 | 334.676 | 608.253 | 167.523 | 79.500 | 89.596 | 457.301 | 176.673 | 132.376 |
| FBL | 39.649 | 55.742 | 12.871 | 6.075 | 10.477 | 49.533 | 18.306 | 13.711 |
| NAP1L1 | 65.018 | 70.666 | 52.466 | 23.949 | 18.156 | 65.716 | 25.047 | 26.867 |
| SRSF1 | 58.006 | 80.199 | 39.536 | 29.719 | 27.952 | 69.178 | 17.731 | 56.601 |
| TCP1 | 51.770 | 134.756 | 30.188 | 30.645 | 25.145 | 38.405 | 23.575 | 54.465 |
| PWP1 | 11.792 | 17.440 | 14.889 | 6.677 | 6.025 | 14.408 | 4.987 | 12.552 |
| APEX1 | 34.372 | 52.860 | 29.789 | 12.301 | 19.745 | 70.716 | 13.554 | 17.773 |
| NME1 | 51.020 | 68.399 | 19.888 | 11.043 | 17.975 | 51.556 | 10.266 | 26.557 |
| EEF1B2 | 147.219 | 248.926 | 85.287 | 58.262 | 74.502 | 269.860 | 95.663 | 152.748 |
| RPS2 | 554.467 | 633.519 | 324.570 | 125.369 | 127.213 | 920.901 | 312.011 | 223.876 |
| RPS3 | 90.963 | 200.581 | 73.532 | 41.663 | 38.281 | 223.115 | 76.257 | 66.797 |
| SRSF3 | 55.567 | 75.810 | 43.234 | 23.492 | 22.046 | 57.933 | 16.463 | 53.636 |
| GOT2 | 24.123 | 34.883 | 21.236 | 13.728 | 12.898 | 19.395 | 12.957 | 31.337 |
| COX5A | 44.642 | 40.390 | 27.993 | 11.621 | 13.712 | 37.057 | 17.420 | 20.130 |
| HNRNPA1 | 10.986 | 12.044 | 8.569 | 3.202 | 3.728 | 14.639 | 4.152 | 4.970 |
| EIF3J | 13.701 | 16.264 | 8.932 | 5.789 | 4.492 | 12.500 | 6.506 | 7.528 |
| HDAC2 | 16.122 | 18.901 | 6.849 | 6.678 | 7.282 | 15.048 | 5.820 | 15.225 |
| CNBP | 56.831 | 69.570 | 36.758 | 18.959 | 17.264 | 44.775 | 22.385 | 32.251 |
| NOLC1 | 20.432 | 44.393 | 24.010 | 6.727 | 9.724 | 30.491 | 18.924 | 22.242 |
| PRDX4 | 59.676 | 86.200 | 22.201 | 12.696 | 14.055 | 55.307 | 16.996 | 16.915 |
| EIF4E | 7.871 | 10.443 | 5.316 | 4.719 | 4.269 | 7.500 | 3.640 | 5.783 |
| RPLP0 | 500.096 | 632.465 | 405.092 | 192.402 | 201.090 | 904.914 | 371.373 | 231.213 |
| ABCE1 | 19.202 | 36.381 | 9.168 | 8.838 | 8.358 | 22.769 | 10.042 | 11.982 |
| LSM7 | 43.420 | 66.028 | 29.601 | 12.082 | 22.912 | 39.968 | 19.402 | 28.653 |
| CLNS1A | 30.985 | 56.695 | 27.223 | 25.322 | 15.404 | 41.430 | 16.254 | 39.127 |
| RSL1D1 | 14.202 | 24.172 | 11.183 | 8.112 | 6.519 | 24.212 | 10.782 | 8.272 |
| HNRNPC | 113.366 | 137.274 | 76.693 | 49.274 | 36.414 | 105.270 | 34.934 | 66.890 |
| H2AFZ | 227.206 | 281.630 | 124.242 | 79.609 | 81.234 | 174.340 | 46.461 | 85.329 |
| EPRS | 29.725 | 32.557 | 28.051 | 20.233 | 10.813 | 28.886 | 13.060 | 19.648 |
| SRPK1 | 19.345 | 52.945 | 16.312 | 9.504 | 10.911 | 16.672 | 11.392 | 20.550 |
| MAD2L1 | 49.770 | 59.489 | 16.850 | 18.750 | 15.589 | 31.595 | 11.307 | 22.110 |
| RPS6 | 415.872 | 1530.287 | 275.273 | 185.470 | 182.728 | 890.036 | 278.583 | 396.643 |
| RRP9 | 19.893 | 19.670 | 8.392 | 2.399 | 3.103 | 15.660 | 7.911 | 7.366 |
| GNL3 | 58.095 | 39.896 | 20.911 | 15.351 | 12.421 | 50.328 | 21.111 | 18.289 |
| SLC19A1 | 5.608 | 10.116 | 3.105 | 1.653 | 3.322 | 8.728 | 2.797 | 2.458 |
| TMEM97 | 39.522 | 51.039 | 15.853 | 13.700 | 15.283 | 58.357 | 9.419 | 14.608 |
| PES1 | 20.826 | 33.842 | 10.856 | 5.609 | 5.581 | 18.437 | 6.666 | 14.980 |
| EXOSC5 | 18.094 | 30.362 | 7.250 | 2.750 | 4.626 | 13.185 | 8.096 | 13.998 |
| NDUFAF4 | 6.751 | 8.893 | 4.193 | 2.457 | 3.594 | 5.460 | 2.714 | 4.204 |
| RABEPK | 8.744 | 14.571 | 4.548 | 1.926 | 3.176 | 9.208 | 3.290 | 6.417 |
| WDR74 | 16.631 | 35.286 | 20.001 | 5.818 | 6.771 | 20.560 | 11.610 | 12.794 |
| PLK4 | 9.571 | 10.671 | 4.599 | 4.541 | 4.423 | 6.838 | 1.898 | 4.715 |
| RCL1 | 11.674 | 20.763 | 4.069 | 3.446 | 2.726 | 11.234 | 4.609 | 6.041 |
| PPRC1 | 6.558 | 14.800 | 3.551 | 1.171 | 2.997 | 7.863 | 5.954 | 2.972 |
| MYC | 0.375 | 0.031 | 0.012 | 0.726 | 0.190 | 0.059 | 0.096 | 0.809 |

TABLE 13
Gene Expression in Neuroblastoma Cell Lines

| | Cell Line | | | | | | | |
|---|---|---|---|---|---|---|---|---|
| Gene | HU.FETAL.BRAIN | IMR32 | IMR5 | KELLY | LAN5 | LAN6 | NB1 | NB16 |
| MYCN | 8.937 | 152.787 | 289.376 | 644.877 | 786.076 | 2.771 | 273.312 | 1.773 |
| MXI1 | 7.589 | 2.713 | 9.829 | 12.417 | 14.203 | 3.931 | 4.723 | 5.313 |
| MPST | 4.076 | 6.289 | 22.435 | 25.194 | 18.790 | 12.192 | 15.291 | 20.075 |
| MAX | 5.642 | 3.517 | 7.116 | 7.375 | 10.090 | 6.883 | 7.520 | 4.398 |
| SMARCA4 | 14.703 | 15.078 | 24.281 | 28.621 | 29.178 | 19.547 | 25.872 | 19.758 |
| SET | 24.949 | 63.172 | 138.895 | 130.513 | 134.025 | 63.794 | 86.567 | 135.728 |
| RAN | 18.450 | 78.151 | 202.757 | 171.237 | 120.032 | 113.312 | 103.035 | 131.296 |
| NPM1 | 16.269 | 238.330 | 798.954 | 858.626 | 205.326 | 163.451 | 269.024 | 674.899 |
| FBL | 10.795 | 16.002 | 63.071 | 81.565 | 29.170 | 18.688 | 29.823 | 53.289 |
| NAP1L1 | 10.402 | 44.521 | 53.536 | 116.150 | 48.411 | 27.104 | 19.377 | 87.861 |
| SRSF1 | 11.392 | 45.149 | 73.127 | 70.990 | 73.551 | 49.209 | 49.215 | 52.011 |
| TCP1 | 22.679 | 52.086 | 161.813 | 116.858 | 71.357 | 23.796 | 55.310 | 137.966 |
| PWP1 | 6.254 | 10.893 | 19.223 | 18.936 | 15.642 | 13.408 | 19.701 | 20.508 |
| APEX1 | 13.620 | 17.288 | 57.070 | 73.264 | 51.480 | 29.863 | 57.939 | 50.207 |
| NME1 | 4.826 | 34.053 | 60.731 | 98.757 | 66.459 | 18.315 | 38.346 | 36.024 |
| EEF1B2 | 54.730 | 118.776 | 293.454 | 202.378 | 204.598 | 107.716 | 169.655 | 253.030 |
| RPS2 | 313.180 | 280.921 | 1388.890 | 1473.079 | 442.038 | 450.411 | 632.388 | 1073.400 |
| RPS3 | 37.558 | 52.836 | 211.758 | 296.795 | 104.097 | 57.131 | 108.832 | 226.228 |
| SRSF3 | 11.626 | 36.120 | 64.501 | 80.308 | 66.980 | 39.207 | 50.527 | 72.193 |
| GOT2 | 10.827 | 10.508 | 27.760 | 29.993 | 41.594 | 17.369 | 34.984 | 20.908 |
| COX5A | 18.247 | 22.799 | 62.134 | 77.767 | 35.845 | 39.190 | 59.953 | 70.198 |
| HNRNPA1 | 2.453 | 5.221 | 12.337 | 11.378 | 7.442 | 7.328 | 8.545 | 9.196 |
| EIF3J | 4.122 | 7.883 | 24.902 | 23.573 | 14.586 | 10.339 | 15.267 | 12.007 |
| HDAC2 | 4.313 | 12.523 | 20.967 | 26.737 | 16.895 | 8.855 | 11.834 | 21.784 |
| CNBP | 11.210 | 27.148 | 61.016 | 97.445 | 52.659 | 40.264 | 39.175 | 68.325 |
| NOLC1 | 6.085 | 17.572 | 69.055 | 39.070 | 37.901 | 23.849 | 37.701 | 45.368 |
| PRDX4 | 5.837 | 17.252 | 65.462 | 76.354 | 62.676 | 31.549 | 41.425 | 50.028 |
| EIF4E | 3.606 | 3.985 | 10.904 | 13.547 | 11.169 | 5.322 | 9.273 | 8.706 |
| RPLP0 | 180.096 | 503.448 | 1198.520 | 1281.980 | 602.731 | 588.309 | 469.627 | 962.850 |
| ABCE1 | 2.968 | 13.558 | 43.890 | 41.760 | 30.447 | 11.900 | 22.272 | 35.889 |
| LSM7 | 14.123 | 21.395 | 57.444 | 60.639 | 40.852 | 29.235 | 57.274 | 46.034 |
| CLNS1A | 14.169 | 20.097 | 66.135 | 59.210 | 49.477 | 18.070 | 37.196 | 40.038 |
| RSL1D1 | 3.692 | 9.994 | 27.430 | 31.263 | 19.104 | 11.017 | 15.463 | 28.190 |
| HNRNPC | 36.212 | 55.039 | 159.326 | 164.688 | 109.139 | 59.790 | 78.901 | 136.915 |
| H2AFZ | 25.376 | 106.930 | 253.609 | 320.392 | 227.570 | 111.491 | 152.085 | 194.739 |
| EPRS | 4.518 | 21.757 | 80.830 | 98.908 | 26.632 | 33.793 | 47.492 | 43.920 |
| SRPK1 | 8.787 | 14.852 | 31.948 | 41.457 | 27.984 | 10.542 | 15.978 | 13.831 |
| MAD2L1 | 0.780 | 26.521 | 52.777 | 63.316 | 77.571 | 16.875 | 34.188 | 36.258 |
| RPS6 | 140.580 | 297.448 | 998.870 | 1146.248 | 562.849 | 208.909 | 553.788 | 658.861 |
| RRP9 | 2.953 | 7.285 | 22.666 | 30.884 | 13.149 | 4.592 | 10.683 | 20.592 |
| GNL3 | 4.892 | 24.927 | 79.642 | 88.163 | 60.336 | 9.748 | 42.126 | 60.193 |
| SLC19A1 | 2.608 | 3.178 | 6.122 | 5.369 | 9.145 | 5.015 | 5.946 | 5.580 |
| TMEM97 | 2.280 | 19.816 | 34.449 | 34.775 | 42.302 | 7.832 | 28.257 | 21.346 |
| PES1 | 6.336 | 9.081 | 21.142 | 42.574 | 22.832 | 14.168 | 17.484 | 29.885 |
| EXOSC5 | 4.238 | 5.770 | 21.173 | 35.409 | 16.528 | 11.884 | 13.631 | 16.300 |
| NDUFAF4 | 2.628 | 4.633 | 7.217 | 11.647 | 7.317 | 1.713 | 4.747 | 7.054 |
| RABEPK | 4.183 | 8.915 | 12.796 | 12.046 | 8.328 | 5.761 | 9.319 | 10.878 |
| WDR74 | 5.419 | 10.190 | 20.315 | 29.982 | 25.173 | 16.885 | 18.694 | 20.459 |
| PLK4 | 0.272 | 5.397 | 10.545 | 9.354 | 12.452 | 5.744 | 7.734 | 9.020 |
| RCL1 | 2.779 | 5.598 | 13.220 | 15.221 | 11.885 | 8.352 | 7.967 | 6.135 |
| PPRC1 | 2.364 | 5.446 | 11.739 | 10.389 | 10.359 | 5.747 | 8.366 | 7.368 |
| MYC | 3.286 | 0.061 | 0.000 | 0.025 | 0.070 | 2.030 | 0.018 | 17.040 |

TABLE 14
Gene Expression in Neuroblastoma Cell Lines

| | Cell Line | | | | | | | |
|---|---|---|---|---|---|---|---|---|
| Gene | NB1643 | NB1691 | NB69 | NBEBC1 | NBLS | NBSD | NGP | NLF |
| MYCN | 395.478 | 108.942 | 0.570 | 8.053 | 31.970 | 280.671 | 700.038 | 41.178 |
| MXI1 | 7.050 | 9.804 | 6.521 | 1.992 | 8.436 | 7.851 | 16.084 | 4.626 |
| MPST | 16.288 | 7.766 | 13.316 | 11.483 | 14.615 | 15.180 | 37.451 | 25.894 |
| MAX | 5.782 | 4.833 | 4.516 | 8.940 | 7.524 | 5.440 | 11.075 | 6.008 |
| SMARCA4 | 23.128 | 19.434 | 25.503 | 16.873 | 29.981 | 25.269 | 38.761 | 24.336 |
| SET | 78.655 | 66.152 | 126.079 | 136.868 | 108.545 | 115.980 | 159.194 | 174.647 |
| RAN | 81.214 | 75.750 | 134.374 | 128.530 | 129.690 | 83.661 | 135.119 | 124.207 |
| NPM1 | 344.213 | 283.493 | 520.328 | 172.879 | 290.033 | 174.317 | 633.101 | 536.492 |
| FBL | 35.961 | 44.882 | 59.380 | 14.099 | 42.532 | 19.114 | 128.548 | 35.819 |
| NAP1L1 | 59.951 | 51.397 | 69.348 | 47.646 | 49.118 | 44.236 | 119.605 | 66.924 |
| SRSF1 | 67.205 | 45.043 | 71.808 | 61.972 | 58.673 | 44.853 | 69.515 | 83.868 |
| TCP1 | 50.952 | 67.903 | 97.093 | 70.977 | 81.985 | 95.451 | 95.373 | 68.020 |
| PWP1 | 13.009 | 17.512 | 27.512 | 16.664 | 12.400 | 16.041 | 26.345 | 9.771 |
| APEX1 | 46.086 | 38.386 | 33.489 | 31.425 | 51.350 | 38.401 | 64.015 | 57.634 |
| NME1 | 53.924 | 15.865 | 91.254 | 29.329 | 39.512 | 22.743 | 61.198 | 51.470 |
| EEF1B2 | 230.727 | 129.882 | 208.373 | 131.733 | 271.322 | 116.874 | 302.958 | 185.460 |
| RPS2 | 778.175 | 633.882 | 1019.556 | 226.423 | 683.637 | 281.817 | 1560.447 | 590.705 |
| RPS3 | 162.933 | 131.120 | 187.741 | 71.989 | 154.110 | 83.618 | 450.528 | 124.086 |
| SRSF3 | 46.463 | 44.675 | 55.682 | 63.375 | 73.050 | 48.882 | 68.234 | 76.693 |
| GOT2 | 19.293 | 29.545 | 38.090 | 24.616 | 31.543 | 26.724 | 47.084 | 20.408 |
| COX5A | 42.743 | 27.792 | 75.156 | 21.845 | 87.663 | 27.973 | 80.267 | 39.209 |
| HNRNPA1 | 11.145 | 9.213 | 11.605 | 6.557 | 11.269 | 7.105 | 13.131 | 6.869 |
| EIF3J | 14.404 | 9.366 | 24.852 | 8.526 | 23.495 | 9.955 | 29.377 | 15.650 |
| HDAC2 | 17.909 | 19.943 | 16.985 | 11.832 | 18.035 | 17.503 | 33.074 | 16.778 |
| CNBP | 32.738 | 29.646 | 63.436 | 52.116 | 47.912 | 33.003 | 81.891 | 55.042 |
| NOLC1 | 34.006 | 18.068 | 53.676 | 16.640 | 40.268 | 20.845 | 37.439 | 27.843 |
| PRDX4 | 40.764 | 61.676 | 66.110 | 36.402 | 34.821 | 39.220 | 97.180 | 60.956 |
| EIF4E | 6.182 | 4.246 | 4.811 | 5.034 | 10.413 | 5.540 | 10.285 | 9.198 |
| RPLP0 | 731.842 | 1301.165 | 836.921 | 395.440 | 816.989 | 420.175 | 1343.204 | 578.184 |
| ABCE1 | 21.347 | 13.528 | 39.084 | 13.530 | 21.452 | 15.096 | 39.391 | 27.804 |
| LSM7 | 31.504 | 29.487 | 56.694 | 31.284 | 72.155 | 37.361 | 85.033 | 64.894 |
| CLNS1A | 32.964 | 25.349 | 47.023 | 46.765 | 57.557 | 41.381 | 81.391 | 47.933 |
| RSL1D1 | 18.171 | 14.112 | 24.445 | 13.626 | 23.160 | 16.583 | 30.095 | 24.793 |
| HNRNPC | 78.280 | 79.461 | 82.854 | 122.780 | 117.717 | 71.388 | 136.953 | 152.445 |
| H2AFZ | 156.199 | 113.922 | 134.246 | 116.199 | 172.637 | 103.219 | 151.166 | 231.424 |
| EPRS | 44.941 | 42.195 | 65.644 | 34.239 | 29.209 | 21.656 | 97.886 | 25.137 |
| SRPK1 | 16.636 | 16.118 | 22.079 | 18.090 | 18.618 | 32.468 | 36.407 | 19.729 |
| MAD2L1 | 35.576 | 21.602 | 25.412 | 23.037 | 38.104 | 28.878 | 37.439 | 48.682 |
| RPS6 | 571.272 | 727.538 | 833.568 | 458.491 | 705.523 | 383.252 | 1494.047 | 977.577 |
| RRP9 | 9.485 | 7.518 | 21.456 | 15.819 | 12.911 | 6.769 | 16.686 | 15.628 |
| GNL3 | 39.119 | 25.077 | 58.937 | 40.853 | 41.276 | 19.567 | 30.549 | 50.108 |
| SLC19A1 | 6.145 | 2.499 | 7.542 | 3.053 | 4.917 | 6.301 | 9.593 | 5.201 |
| TMEM97 | 33.486 | 24.589 | 38.714 | 25.596 | 21.549 | 31.739 | 38.271 | 26.739 |
| PES1 | 19.837 | 9.790 | 31.823 | 28.206 | 23.052 | 10.571 | 42.033 | 30.740 |
| EXOSC5 | 10.634 | 11.092 | 26.016 | 11.567 | 22.966 | 8.499 | 34.984 | 17.533 |
| NDUFAF4 | 4.423 | 6.528 | 7.253 | 3.841 | 5.130 | 4.370 | 11.826 | 5.600 |
| RABEPK | 5.531 | 5.302 | 5.098 | 7.386 | 7.494 | 6.587 | 16.854 | 16.607 |
| WDR74 | 14.843 | 9.561 | 28.162 | 17.464 | 21.796 | 17.716 | 27.897 | 27.978 |
| PLK4 | 8.053 | 4.821 | 4.445 | 6.631 | 10.431 | 5.713 | 8.999 | 11.918 |
| RCL1 | 8.808 | 5.786 | 12.737 | 13.859 | 11.250 | 6.609 | 9.513 | 7.593 |
| PPRC1 | 9.473 | 4.690 | 11.450 | 3.420 | 7.492 | 4.474 | 9.859 | 7.438 |
| MYC | 0.022 | 0.122 | 165.137 | 0.115 | 0.191 | 0.011 | 0.118 | 0.134 |

TABLE 15
Gene Expression in Neuroblastoma Cell Lines

| | Cell Line | | | | | | | |
|---|---|---|---|---|---|---|---|---|
| Gene | NMB | RPE1 | SHSY5Y | SKNAS | SKNBE2 | SKNBE2C | SKNDZ | SKNFI |
| MYCN | 208.079 | 0.085 | 1.451 | 0.046 | 695.209 | 520.553 | 256.233 | 18.155 |
| MXI1 | 6.431 | 4.625 | 5.327 | 5.760 | 16.499 | 7.278 | 9.237 | 7.845 |
| MPST | 14.616 | 10.892 | 20.426 | 14.359 | 41.759 | 27.022 | 34.107 | 10.317 |
| MAX | 6.563 | 7.467 | 7.821 | 9.033 | 8.825 | 6.165 | 5.062 | 9.029 |
| SMARCA4 | 19.479 | 9.593 | 26.997 | 16.545 | 33.665 | 25.484 | 29.151 | 7.832 |
| SET | 98.419 | 96.177 | 127.018 | 89.213 | 133.778 | 120.018 | 161.399 | 108.930 |
| RAN | 84.081 | 122.168 | 134.921 | 145.570 | 122.629 | 171.855 | 174.951 | 125.912 |
| NPM1 | 210.718 | 342.794 | 269.738 | 365.721 | 678.646 | 951.955 | 638.103 | 112.344 |
| FBL | 18.353 | 28.950 | 41.764 | 33.006 | 28.614 | 86.846 | 71.884 | 15.564 |
| NAP1L1 | 57.160 | 64.678 | 77.416 | 99.280 | 80.669 | 129.184 | 115.709 | 72.438 |
| SRSF1 | 44.756 | 44.705 | 63.478 | 53.877 | 60.656 | 57.290 | 74.446 | 54.660 |
| TCP1 | 41.944 | 74.795 | 45.996 | 57.677 | 106.328 | 127.597 | 117.059 | 84.311 |
| PWP1 | 9.760 | 16.193 | 19.601 | 17.064 | 22.417 | 24.844 | 17.720 | 22.311 |
| APEX1 | 34.085 | 37.280 | 38.752 | 41.385 | 77.729 | 62.752 | 79.463 | 38.972 |
| NME1 | 28.882 | 24.668 | 45.357 | 41.571 | 61.601 | 74.222 | 59.172 | 25.076 |
| EEF1B2 | 112.177 | 147.368 | 173.404 | 145.262 | 212.614 | 272.165 | 263.583 | 105.128 |
| RPS2 | 426.039 | 770.216 | 1022.110 | 576.564 | 1259.885 | 1584.527 | 942.869 | 224.013 |
| RPS3 | 85.620 | 121.718 | 157.636 | 85.077 | 400.948 | 284.136 | 312.008 | 89.329 |
| SRSF3 | 37.673 | 53.003 | 73.306 | 51.324 | 55.697 | 66.941 | 80.043 | 54.763 |
| GOT2 | 22.881 | 29.405 | 25.066 | 28.442 | 24.264 | 31.983 | 59.872 | 28.548 |
| COX5A | 13.302 | 34.231 | 33.548 | 41.172 | 76.563 | 105.154 | 75.938 | 43.906 |
| HNRNPA1 | 6.916 | 7.216 | 14.565 | 6.263 | 11.215 | 17.047 | 13.812 | 5.658 |
| EIF3J | 8.792 | 13.337 | 13.395 | 15.157 | 24.423 | 21.150 | 29.863 | 21.673 |
| HDAC2 | 10.380 | 12.498 | 17.592 | 6.526 | 26.679 | 23.361 | 32.109 | 10.879 |
| CNBP | 35.940 | 28.435 | 48.867 | 57.758 | 70.622 | 84.649 | 53.495 | 40.897 |
| NOLC1 | 34.312 | 26.127 | 29.051 | 29.041 | 45.513 | 47.125 | 50.661 | 35.449 |
| PRDX4 | 31.469 | 46.293 | 45.859 | 50.307 | 79.764 | 130.470 | 72.664 | 35.515 |
| EIF4E | 5.677 | 8.980 | 8.955 | 5.916 | 9.347 | 11.221 | 9.381 | 9.983 |
| RPLP0 | 556.801 | 774.252 | 1030.430 | 1101.970 | 1133.312 | 1559.634 | 844.967 | 535.573 |
| ABCE1 | 16.931 | 27.624 | 21.043 | 36.249 | 39.635 | 39.768 | 27.874 | 20.547 |
| LSM7 | 27.083 | 24.766 | 58.845 | 49.404 | 75.899 | 58.881 | 52.470 | 36.379 |
| CLNS1A | 38.426 | 26.019 | 54.480 | 39.324 | 87.667 | 76.846 | 78.641 | 67.280 |
| RSL1D1 | 20.007 | 11.502 | 15.909 | 23.842 | 29.892 | 31.013 | 23.716 | 16.210 |
| HNRNPC | 75.643 | 91.783 | 110.254 | 134.075 | 131.462 | 144.708 | 136.610 | 88.363 |
| H2AFZ | 133.199 | 300.753 | 356.196 | 140.999 | 176.603 | 302.064 | 210.055 | 188.604 |
| EPRS | 18.994 | 26.697 | 37.829 | 52.665 | 89.007 | 97.193 | 37.645 | 24.053 |
| SRPK1 | 20.213 | 21.594 | 19.899 | 25.215 | 30.508 | 32.608 | 35.306 | 18.142 |
| MAD2L1 | 32.170 | 42.088 | 53.242 | 45.961 | 45.730 | 50.097 | 65.573 | 57.392 |
| RPS6 | 498.226 | 661.175 | 681.900 | 440.453 | 1339.403 | 928.975 | 520.400 | 534.475 |
| RRP9 | 7.699 | 7.670 | 9.305 | 8.796 | 16.130 | 16.024 | 17.820 | 13.872 |
| GNL3 | 20.833 | 25.810 | 49.609 | 33.737 | 42.674 | 37.691 | 50.909 | 38.624 |
| SLC19A1 | 5.139 | 2.936 | 5.758 | 7.451 | 6.121 | 4.742 | 5.009 | 5.339 |
| TMEM97 | 26.456 | 7.626 | 16.796 | 10.144 | 27.174 | 26.457 | 58.973 | 24.463 |
| PES1 | 11.787 | 18.276 | 20.033 | 21.244 | 22.512 | 29.951 | 24.084 | 22.809 |
| EXOSC5 | 12.371 | 5.873 | 13.855 | 20.233 | 17.551 | 28.498 | 31.397 | 14.761 |
| NDUFAF4 | 3.298 | 2.520 | 5.139 | 7.105 | 8.148 | 9.231 | 8.826 | 4.621 |
| RABEPK | 4.898 | 7.506 | 6.428 | 10.803 | 17.277 | 10.899 | 12.057 | 9.239 |
| WDR74 | 15.692 | 8.365 | 18.033 | 26.597 | 31.794 | 13.403 | 34.750 | 21.016 |
| PLK4 | 6.018 | 9.744 | 14.632 | 8.814 | 8.362 | 8.770 | 13.374 | 10.956 |
| RCL1 | 5.811 | 2.926 | 7.593 | 6.866 | 10.737 | 9.348 | 6.272 | 8.191 |
| PPRC1 | 5.765 | 5.290 | 5.621 | 5.508 | 9.086 | 8.061 | 9.780 | 4.982 |
| MYC | 0.010 | 18.582 | 15.628 | 10.235 | 0.239 | 0.061 | 0.019 | 1.749 |

TABLE 16
Gene Expression in Neuroblastoma Cell Lines

| | Cell Lines | | |
|---|---|---|---|
| Gene | SKNSH | SMSKAN | SMSSAN |
| MYCN | 4.962 | 301.818 | 1052.545 |
| MXI1 | 12.631 | 11.047 | 15.801 |
| MPST | 18.931 | 14.775 | 18.995 |
| MAX | 7.981 | 6.933 | 8.472 |
| SMARCA4 | 16.265 | 25.926 | 24.979 |
| SET | 77.062 | 111.486 | 183.833 |
| RAN | 58.286 | 87.561 | 237.991 |
| NPM1 | 239.060 | 406.947 | 330.598 |
| FBL | 32.735 | 26.843 | 65.160 |
| NAP1L1 | 47.465 | 54.066 | 99.898 |
| SRSF1 | 54.479 | 63.431 | 73.632 |
| TCP1 | 47.467 | 59.159 | 93.453 |
| PWP1 | 12.149 | 14.267 | 33.785 |
| APEX1 | 29.060 | 52.581 | 60.849 |
| NME1 | 27.523 | 35.736 | 89.419 |
| EEF1B2 | 212.631 | 269.167 | 237.280 |
| RPS2 | 849.440 | 418.960 | 901.235 |
| RPS3 | 250.386 | 134.312 | 159.796 |
| SRSF3 | 36.092 | 49.417 | 88.188 |
| GOT2 | 13.871 | 24.089 | 38.093 |
| COX5A | 32.410 | 30.054 | 73.599 |
| HNRNPA1 | 11.208 | 10.391 | 14.227 |
| EIF3J | 9.492 | 9.531 | 21.418 |
| HDAC2 | 12.957 | 12.957 | 31.825 |
| CNBP | 32.464 | 39.917 | 74.971 |
| NOLC1 | 28.407 | 26.036 | 46.566 |
| PRDX4 | 57.399 | 46.150 | 73.903 |
| EIF4E | 6.603 | 6.950 | 13.808 |
| RPLP0 | 840.059 | 709.218 | 1229.434 |
| ABCE1 | 14.870 | 20.396 | 38.386 |
| LSM7 | 40.744 | 49.376 | 71.095 |
| CLNS1A | 54.215 | 39.160 | 55.652 |
| RSL1D1 | 18.946 | 20.151 | 22.244 |
| HNRNPC | 67.634 | 90.337 | 155.693 |
| H2AFZ | 91.297 | 159.725 | 416.078 |
| EPRS | 38.494 | 32.591 | 62.475 |
| SRPK1 | 16.585 | 19.425 | 34.762 |
| MAD2L1 | 20.466 | 36.290 | 86.140 |
| RPS6 | 956.383 | 830.900 | 715.863 |
| RRP9 | 9.199 | 13.021 | 19.128 |
| GNL3 | 45.487 | 40.781 | 78.680 |
| SLC19A1 | 4.654 | 7.325 | 5.816 |
| TMEM97 | 15.576 | 75.893 | 42.636 |
| PES1 | 18.075 | 18.394 | 22.737 |
| EXOSC5 | 14.283 | 7.771 | 19.472 |
| NDUFAF4 | 3.059 | 3.543 | 9.274 |
| RABEPK | 5.852 | 7.887 | 13.711 |
| WDR74 | 18.898 | 19.967 | 23.982 |
| PLK4 | 4.303 | 9.639 | 15.168 |
| RCL1 | 9.711 | 9.909 | 13.756 |
| PPRC1 | 4.958 | 8.188 | 7.105 |
| MYC | 25.718 | 0.080 | 0.032 |

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

Each of the following references is herein incorporated by reference in their entirety.
1 Sturgeon, K. M. et al. A population-based study of cardiovascular disease mortality risk in US cancer patients. Eur Heart J, doi:10.1093/eurheartj/ehz766 (2019).
2 Du, S., Huang, Y., Jin, H. & Wang, T. Protective Mechanism of Hydrogen Sulfide against Chemotherapy-Induced Cardiotoxicity. Front Pharmacol 9, 32, doi:10.3389/fphar.2018.00032 (2018).
3 Chow, E. J. et al. Pediatric cardio-oncology: epidemiology, screening, prevention, and treatment. Cardiovasc Res 115, 922-934, doi:10.1093/cvr/cvz031 (2019).
4 Knowles, D. A. et al. Determining the genetic basis of anthracycline-cardiotoxicity by molecular response QTL mapping in induced cardiomyocytes. Elife 7, doi:10.7554/eLife.33480 (2018).
5 McGowan, J. V. et al. Anthracycline Chemotherapy and Cardiotoxicity. Cardiovasc Drugs Ther 31, 63-75, doi:10.1007/s10557-016-6711-0 (2017).
6 Amoroso, L. et al. Topotecan-Vincristine-Doxorubicin in Stage 4 High-Risk Neuroblastoma Patients Failing to Achieve a Complete Metastatic Response to Rapid COJEC: A SIOPEN Study. Cancer Res Treat 50, 148-155, doi:10.4143/crt.2016.511 (2018).
7 Vittorio, 0. et al. Doxorubicin synergism and resistance reversal in human neuroblastoma BE(2)C cell lines: An in vitro study with dextran-catechin nanohybrids. Eur J Pharm Biopharm 122, 176-185, doi:10.1016/j.ejpb.2017.11.005 (2018).
8 Bean, J. F. et al. Glycolysis inhibition and its effect in doxorubicin resistance in neuroblastoma. J Pediatr Surg 49, 981-984; discussion 984, doi:10.1016/j jpedsurg.2014.01.037 (2014).
9 Flahaut, M. et al. Molecular cytogenetic characterization of doxorubicin-resistant neuroblastoma cell lines: evidence that acquired multidrug resistance results from a unique large amplification of the 7q21 region. Genes Chromosomes Cancer 45, 495-508, doi:10.1002/gcc.20312 (2006).
10 Kotchetkov, R. et al. Development of resistance to vincristine and doxorubicin in neuroblastoma alters malignant properties and induces additional karyotype changes: a preclinical model. Int J Cancer 104, 36-43, doi:10.1002/ijc.10917 (2003).
11 Lautz, T. B. et al. The effect of vorinostat on the development of resistance to doxorubicin in neuroblastoma. PLoS One 7, e40816, doi:10.1371/journal.pone.0040816 (2012).
12 Rebbaa, A., Chou, P. M. & Mirkin, B. L. Factors secreted by human neuroblastoma mediated doxorubicin resistance by activating STAT3 and inhibiting apoptosis. Mol Med 7, 393-400 (2001).
13 Wenningmann, N., Knapp, M., Ande, A., Vaidya, T. R. & Ait-Oudhia, S. Insights into Doxorubicin-induced Cardiotoxicity: Molecular Mechanisms, Preventive Strategies, and Early Monitoring. Mol Pharmacol 96, 219-232, doi:10.1124/mo1.119.115725 (2019).
14 Ganatra, S. et al. Upfront dexrazoxane for the reduction of anthracycline-induced cardiotoxicity in adults with preexisting cardiomyopathy and cancer: a consecutive case series. Cardio-Oncology 5, 1, doi:10.1186/s40959-019-0036-7 (2019).
15 Harenza, J. L. et al. Transcriptomic profiling of 39 commonly-used neuroblastoma cell lines. Sci Data 4, 170033, doi:10.1038/sdata.2017.33 (2017).
16 Szczesny, B. et al. AP39, a novel mitochondria-targeted hydrogen sulfide donor, stimulates cellular bioenergetics, exerts cytoprotective effects and protects against the loss of mitochondrial DNA integrity in oxidatively stressed endothelial cells in vitro. Nitric oxide: biology and chemistry 41, 120-130, doi:10.1016/j.niox.2014.04.008 (2014).
17 Peirce, S. K. et al. The PI-3 kinase-Akt-MDM2-survivin signaling axis in high-risk neuroblastoma: a target for PI-3 kinase inhibitor intervention. Cancer Chemother Pharmacol 68, 325-335, doi:10.1007/s00280-010-1486-7 (2011).
18 Keshelava, N., Seeger, R. C., Groshen, S. & Reynolds, C. P. Drug resistance patterns of human neuroblastoma cell lines derived from patients at different phases of therapy. Cancer Res 58, 5396-5405 (1998).
19 Stokes, E. et al. Efflux inhibition by H₂S confers sensitivity to doxorubicin-induced cell death in liver cancer cells. Life Sci 213, 116-125, doi:10.1016/j.lfs.2018.10.031 (2018).
20 Covarrubias, A. E. et al. AP39, a Modulator of Mitochondrial Bioenergetics, Reduces Antiangiogenic Response and Oxidative Stress in Hypoxia-Exposed Trophoblasts: Relevance for Preeclampsia Pathogenesis. Am J Pathol 189, 104-114, doi:10.1016/j.ajpath.2018.09.007 (2019).
21 Chatzianastasiou, A. et al. Cardioprotection by H₂S Donors: Nitric Oxide-Dependent and Independent Mechanisms. J Pharmacol Exp Ther 358, 431-440, doi:10.1124/jpet.116.235119 (2016).
22 Ikeda, K. et al. Mitochondria-targeted hydrogen sulfide donor AP39 improves neurological outcomes after cardiac arrest in mice. Nitric oxide: biology and chemistry 49, 90-96, doi:10.1016/j.niox.2015.05.001 (2015).
23 Tomasova, L. et al. Effects of AP39, a novel triphenylphosphonium derivatised anethole dithiolethione hydrogen sulfide donor, on rat haemodynamic parameters and chloride and calcium Cav3 and RyR2 channels. Nitric oxide: biology and chemistry 46, 131-144, doi:10.1016/j.niox.2014.12.012 (2015).
24 Zhu, C. et al. Supplementing preservation solution with mitochondria-targeted H2 S donor AP39 protects cardiac grafts from prolonged cold ischemia-reperfusion injury in heart transplantation. Am J Transplant, doi:10.1111/ajt.15539 (2019).
25 Xie, Z. Z., Liu, Y. & Bian, J. S. Hydrogen Sulfide and Cellular Redox Homeostasis. Oxid Med Cell Longev 2016, 6043038, doi:10.1155/2016/6043038 (2016).
26 Bos, E. M., van Goor, H., Joles, J. A., Whiteman, M. & Leuvenink, H. G. Hydrogen sulfide: physiological properties and therapeutic potential in ischaemia. Br J Pharmacol 172, 1479-1493, doi:10.1111/bph.12869 (2015).
27 Karwi, Q. G., Bice, J. S. & Baxter, G. F. Pre- and postconditioning the heart with hydrogen sulfide (H₂S) against ischemia/reperfusion injury in vivo: a systematic review and meta-analysis. Basic Res Cardiol 113, 6, doi:10.1007/s00395-017-0664-8 (2018).
28 Kanagy, N. L. & Kevil, C. G. The pleiotropic effects of hydrogen sulfide. Am J Physiol Heart Circ Physiol 314, H1-H2, doi:10.1152/ajpheart.00613.2017 (2018).
29 Wang, L. et al. Protection against doxorubicin-induced myocardial dysfunction in mice by cardiac-specific expression of carboxyl terminus of hsp70-interacting protein. Sci Rep 6, 28399, doi:10.1038/srep28399 (2016).
30 Blaheta, R. A. et al. Chemoresistance induces enhanced adhesion and transendothelial penetration of neuroblastoma cells by down-regulating NCAM surface expression. BMC Cancer 6, 294, doi:10.1186/1471-2407-6-294 (2006).
31 Casique-Aguirre, D. et al. KRas4B-PDE6delta complex stabilization by small molecules obtained by virtual screening affects Ras signaling in pancreatic cancer. BMC Cancer 18, 1299, doi:10.1186/s12885-018-5142-7 (2018).
32 Hsu, C. L. et al. Unveiling MYCN regulatory networks in neuroblastoma via integrative analysis of heterogeneous genomics data. Oncotarget 7, 36293-36310, doi:10.18632/oncotarget.9202 (2016).
33 Faustino-Rocha, A. et al. Estimation of rat mammary tumor volume using caliper and ultrasonography measurements. Lab Anim (NY) 42, 217-224, doi:10.1038/laban.254 (2013).
34 Morton, C. L. & Houghton, P. J. Establishment of human tumor xenografts in immunodeficient mice. Nat Protoc 2, 247-250, doi:10.1038/nprot.2007.25 (2007).
35 An, L. et al. UVRAG Deficiency Exacerbates Doxorubicin-Induced Cardiotoxicity. Sci Rep 7, 43251, doi:10.1038/srep43251 (2017).
36 Pauli, C. et al. Personalized In Vitro and In Vivo Cancer Models to Guide Precision Medicine. Cancer Discov 7, 462-477, doi:10.1158/2159-8290.CD-16-1154 (2017).
37 Hanna, C., Kwok, L., Finlay-Schultz, J., Sartorius, C. A. & Cittelly, D. M. Labeling of Breast Cancer Patient-derived Xenografts with Traceable Reporters for Tumor Growth and Metastasis Studies. J Vis Exp, doi:10.3791/54944 (2016).
38 Zhu, W., Shou, W., Payne, R. M., Caldwell, R. & Field, L. J. A mouse model for juvenile doxorubicin-induced cardiac dysfunction. Pediatr Res 64, 488-494, doi:10.1203/PDR.0b013e318184d732 (2008).
39 Moye, L. Statistical Methods for Cardiovascular Researchers. Circ Res 118, 439-453, doi:10.1161/CIRCRESAHA.115.306305 (2016).
40 Katerji, M., Filippova, M. & Duerksen-Hughes, P. Approaches and Methods to Measure Oxidative Stress in Clinical Samples: Research Applications in the Cancer Field. Oxid Med Cell Longev 2019, 1279250, doi:10.1155/2019/1279250 (2019).
41 Burridge, P. W. et al. Human induced pluripotent stem cell-derived cardiomyocytes recapitulate the predilection of breast cancer patients to doxorubicin-induced cardiotoxicity. Nat Med 22, 547-556, doi:10.1038/nm.4087 (2016).
42 Luongo, T. S. et al. The mitochondrial Na(+)/Ca(2+) exchanger is essential for Ca(2+) homeostasis and viability. Nature 545, 93-97, doi:10.1038/nature22082 (2017).
43 Shen, X. et al. Measurement of plasma hydrogen sulfide in vivo and in vitro. Free Radic Biol Med 50, 1021-1031, doi:10.1016/j.freeradbiomed.2011.01.025 (2011).
44 Shen, X., Chakraborty, S., Dugas, T. R. & Kevil, C. G. Hydrogen sulfide measurement using sulfide dibimane: critical evaluation with electrospray ion trap mass spectrometry. Nitric oxide: biology and chemistry 41, 97-104, doi:10.1016/j.niox.2014.06.002 (2014).
45 Shen, X., Peter, E. A., Bir, S., Wang, R. & Kevil, C. G. Analytical measurement of discrete hydrogen sulfide pools in biological specimens. Free Radic Biol Med 52, 2276-2283, doi:10.1016/j.freeradbiomed.2012.04.007 (2012).
46 Veeranki, S. et al. Moderate intensity exercise prevents diabetic cardiomyopathy associated contractile dysfunction through restoration of mitochondrial function and connexin 43 levels in db/db mice. J Mol Cell Cardiol 92, 163-173, doi:10.1016/j.yjmcc.2016.01.023 (2016).
47 Krishnamurthy, P. et al. Interleukin-10 deficiency impairs bone marrow-derived endothelial progenitor cell survival and function in ischemic myocardium. Circ Res 109, 1280-1289, doi:10.1161/CIRCRESAHA.111.248369 (2011).
48 Conesa, A. et al. A survey of best practices for RNA-seq data analysis. Genome Biol 17, 13, doi:10.1186/s13059-016-0881-8 (2016).
49 Long, C. P. & Antoniewicz, M. R. High-resolution (13)C metabolic flux analysis. Nat Protoc 14, 2856-2877, doi:10.1038/s41596-019-0204-0 (2019).

1. A method of treating doxorubicin-mediated organ toxicity, comprising:
administering AP39 to a subject in need thereof.

2. The method of claim 1, wherein the organ is the heart.

3. The method of claim 1, wherein AP39 is administered at a dose of between about 0.321 mg/kg to about 0.642 mg/kg.

4. The method of claim 1, wherein AP39 is administered at a dose of at least about 20 micromolar.

5. A method of treating cancer, comprising:
administering AP39 to a subject in need thereof; and
administering thiolactate to the subject in need thereof.

6. The method of claim 5, further comprising administering doxorubicin to the subject in need thereof.

7. The method of claim 5, wherein the cancer is lung cancer, neural cancer, breast cancer, or renal cancer.

8. A method of predicting and treating doxorubicin-resistant cancer, comprising:
(a) determining expression of Myc-regulated genes and MPST transcription levels;
(b) classifying the cancer as doxorubicin resistant when either Myc transcription is higher than IMR-32 transcription or MPST transcription levels are above a predetermined amount; and
(c) administering an effective amount of AP39.

9. The method of claim 8, wherein the cancer is neuroblastoma.
